(19) United States
(12) Patent Application Publication
(10) Pub. No.: US 2024/0132819 A1
(43) Pub. Date: Apr. 25, 2024

(54) CELL NICHE ENGINEERING PLATFORM, MULTIPLEXED BIOCHIPS RESULTING THEREFROM AND METHODS OF USE THEREOF

(71) Applicant: The University of Hong Kong, Hong Kong (CN)

(72) Inventors: Pui Barbara CHAN, Hong Kong (CN); Chi Hung YIP, Hong Kong (CN); Abigail Dee CHEN, Hong Kong (CN); Xinna WANG, Hong Kong (CN); Nan HUANG, Hong Kong (CN)

(21) Appl. No.: 18/264,695

(22) PCT Filed: Feb. 7, 2022
(86) PCT No.: PCT/CN2022/075402

§ 371 Date: Aug. 8, 2023

Related U.S. Application Data
(60) Provisional application No. 63/146,779, filed on Feb. 8, 2021.
(60) Provisional application No. 63/147,270, filed on Feb. 9, 2021.

(51) Int. Cl. C12M 1/32; C12M 1/42

(52) U.S. Cl. CPC C12M 23/12; C12M 35/08

(57) ABSTRACT

Provided are cell niche engineering platform which represents a valuable in vitro tool for investigating physiological and pathological cellular activities, an all-in-one technology to engineer cell niche (particularly soluble cell niche factors) with retained bioactivities, a mask-free, non-contact, biocompatible and multiphoton-based microfabrication and micropatterning method for engineering a spatially and quantitatively controllable soluble proteins/bioactive factors and cell-cell adhesion molecules. An universal cell niche engineering platform is provided that contributes to reconstituting heterogeneous native soluble cell niche for signal transduction modeling and drug screening studies.

### FIELD OF THE INVENTION

The invention is generally directed to a biochip system used for cell niche investigation and engineering and cell niche biochips, and methods of making and using the same.

### BACKGROUND OF THE INVENTION

Cell niche is the local microenvironment that regulates the fate and functions of resident cells in native tissues. Niche factors represent the constituent components of the cell niche, including but not limited to the mechanical properties, extracellular matrix (ECM), soluble factors, cell-cell interaction molecules and topological features of the local microenvironment. These cell niche factors are specific to tissue and cell types and act collectively to determine the fate, including but are not limited to phenotype maintenance, adhesion, migration, proliferation, differentiation and apoptosis of the cells.

For decades, in research, pharmaceutical and biotechnology industries, cells are routinely cultured as 2D monolayers on flat and rigid culture dishes made of plastics or glass, owing to their easy handling and relatively low cost. However, it has been realized that this artificial environment is significantly different from that of their native microenvironment in tissues in many aspects including but are not limited to mechanical compliance, the topological features, the ECM components, the soluble factors and the cell-cell interaction molecules. As a result, the fate and the behavior of the in vitro cultured cells are dramatically different from that in native tissues in many aspects including but are not limited to morphology, adhesion, phenotype maintenance, differentiation, proliferation, cytoskeleton organization, migration, survival and responses to drugs and toxic agents. Taking phenotype maintenance as an example. A phenomenon called de-differentiation upon culturing as 2D monolayers on flat and rigid culture plates demonstrates the negative impact of the artificial or non-physiological culture environment, on maintaining the phenotype and hence the fate of the cells. Specifically, many cell types including but are not limited to chondrocytes, hepatocytes, nucleus pulposus cells (NPCs), and tumor cells, lost their physiological phenotype, which is collectively defined as morphology, expression of phenotype markers in gene and protein levels, cellular activities such as migratory property, proliferation capability and differentiation status, and cellular functions such as secretion of certain soluble factors and deposition of certain ECM components, upon culturing as 2D monolayers on flat and rigid culture plates. As a result, these cultured cells cannot maintain their physiological phenotype, in another word, are de-differentiated, before being used in downstream investigations and applications including but are not limited to basic science investigations, drug screening, toxicity tests, tissue engineering and regenerative medicine, and thus ruining the downstream outcomes.

Using dedifferentiated cells with lost phenotype can lead to irrelevant results and wastes of research resources. Different approaches used for phenotypic maintenance include reconstituting the native cell niche using different 3D models such as cell pellet, alginate beads, collagen microspheres, poly-lactic acid (PLA) and so on in the NPC example. However, the optimal native cell niche is often complex with multi-factorial niche signals and a single material or the 3D dimensionality cannot adequately re-capitulate the best combination of cell niche factors for phenotype maintenance. Therefore, systemic studies on screening the ability of multiplex cell niche factors, singly or in combination, in maintaining the NPC phenotype in cultures, is crucial before defining and reconstituting the optimal combinatory cell niche for phenotype maintenance but this is far from elucidation. As a result, high throughput screening platforms for multiplex cell niche factors such as biochips for multiplex cell niche factor screening and biochips for optimal phenotype maintenance are needed

In order to fabricate or produce these biochips, microfabrication and micropatterning technologies are necessary. Most of the conventional technologies are either for microfabrication such as electrospinning and replica remolding, or for micropatterning such as adsorption coating and micro-contact printing. Microfabrication technologies usually work in conjunction with micropatterning technologies to achieve microfabrication of surface functionalized microstructures. However, intrinsic constraints of these technologies exist. For examples, microfabrication technologies such as replica molding and electrospinning have difficulties in fabricating arbitrary microstructure such as those with distinct topological, mechanical and chemical features or even overhanging structures at sufficient resolutions. Micropatterning technologies such as adsorption coating and micro-contact printing (stamp transfer) can hardly functionalize the surface of 3D tiny microstructures with spatial and quantitative heterogeneity at sufficient resolution and precision. 3D printing-based technologies such as micro-inkjet printing can provide both microfabrication and micropatterning functions in the same platform. However, the typical resolution of micro-inkjet printing is from tens to hundreds of microns that is insufficient for cell niche engineering. Moreover, micro-inkjet printing can hardly fabricate microstructures with sufficient complexity and arbitrariness owing to the intrinsic physical limits on its ability to add structures or coatings inside or below a pre-fabricated layer.

Conventional microfabrication platforms including microcontact printing and electrospinning have been used to reconstitute cell niches, to list a few examples, the 2D microarray presented in US20150057184A1 and WO2013037836A1 and the fibrous meshwork by electrospinning or encapsulation in US20160355780A1, KR20160005122A and KR101720378B1. Several studies also reported topological cue screening such as BSSA (WO2006114097A2), TopoChip (US20110009282A1) and MARC. However, these technologies are not able to reconstitute the heterogenous cell microenvironment, such as micropatterning mechanical and biochemical signal heterogeneity in the same microfabrication system. The microstructure achievable are mostly 2D ones instead of complex architecture. As a result, there is still a need for a single robust cell niche engineering or programming technology that is able to microfabricate and micropattern microstructures with independently, quantitatively and spatially controllable features, homogeneous or heterogeneous, singly or in combination.

In engineering or programming cell niche, constructing biochips by incorporating multiple cell niche factors, including but are not limited to the mechanical compliance, topological features, ECM components, soluble factors and cell-cell interaction molecules, in the same platform in an independent and controllable manner, using materials that are biocompatible such as protein-based materials, is necessary. With respect to soluble bioactive factors like proteins, constructing protein chip is complex and challenging because proteins are easily denatured upon the chemical treatment and immobilization. A major concern for crosslinking strategies is the possible denaturation of protein caused by the covalent bonding through physical, chemical or photochemical crosslinking reactions, as well as the randomly oriented immobilization of proteins, limiting its application in reconstituting the protein-protein interactions present in native tissues in vitro for cell niche engineering purpose. Therefore, there is a need for improved methods of attaching soluble proteins onto microstructure surfaces, while retaining the biological activity of the protein.

A primary goal of cell niche engineering or programming is to manipulate and control the cell fate with predictable outcomes. Apart from phenotype maintenance in the NPCs example, other types of cell fate, particularly those specific to stem cells, such as manipulating asymmetric cell division (ACD) orientation in embryonic stem cells (ESCs) as an example, are also difficult to achieve. What is even more challenging is precise engineering of the cell niche to manipulate the cell fate down to a single stem cell level. Given the complexity of local niche signals and their importance in defining cell fate specifications, and the fact that differential cell fates of stem cells are mediated through proper division polarity and ACD orientation, understanding the attributes of individual local niche factors in affecting cell division polarity and ACD orientation is critical for developing strategies to manipulate cell fates through engineering local niche signals of stem cells in single cell precision or resolution. Nevertheless, most existing studies manipulate stem cell fate via in vivo models where multiple niche signals including geometry, matrix and neighbor cells co-exist and cross-talk, making it difficult to delineate the contribution of individual cell niche factors and their interactions. Moreover, these models are usually genetically manipulated to over-express certain regulatory proteins. As a result, spatially and quantitatively controlling individual local niche factors to recapitulate the complex and anisotropic cellular microenvironment such as the apico-basal and the anterior-posterior niches for cell fate manipulation is not possible. There is a need for methods, devices and end products which are able to screen for the cell niche factors and then reconstitute the optimal cell niche factors, which can be relevant in vivo, in single cell resolution, so as to manipulate the fate of a single stem cell in vitro.

A systematic and high-throughput optimal cell niche engineering or programming platform is provided.

A method of making biochips incorporating multiplex individual niche factors or combinatory niches, either mimic the in vivo environment of or provide an artificial microenvironment to a single cell or group of cells (test cell or cells), that give rise to optimal outcomes or fate of the cells, is disclosed.

Biochips incorporating niche factors are also provided.

Methods of incorporating soluble factors into a micro niche are also provided.

A single cell 3D micro-niche platform with controllable and engineerable local cell niche factors is also provided.

Methods of using biochips incorporating cell niche factors are also provided.

### SUMMARY OF THE INVENTION

A method of making biochips incorporation optimal niche factors that mimic the in vivo environment of a cell or group of cells (test cell or cells) is disclosed and biochips incorporating cell(s)-specific optimal niche factors. Cell niche factors include, but are not limited to topological factors, mechanical factors, extracellular matrix factors, cell-cell adhesion molecules, and soluble proteins/bioactive factors. Exemplary topological factors include, but are not limited to factors such as flat matrix (BSA/FM), micro-pillar array (MPA), fiber-bead microstructure (FB), thick grating (TkG), thin grating (TnG), parallel grating hierarchy (GHpl), perpendicular grating hierarchy (GHpp), convex (Cv) and concave (Cc).

The method is a systematic and high-throughput cell niche engineering platform which results in the incorporation of optimal factors into a biochip to provide multiplex protein biochips using as a preferred printing method, multiphoton micropatterning and microfabrication (MMM). Using a two stage screening process, optimal combination of cell niche factors required to manipulate the cell fate, in this case, maintain the phenotype of a test cell or cells, in culture are identified.

The first phase includes microfabricating protein microstructures incorporating individual niche factors identified as relevant for a particular test cell type/types in vivo (for example, by selecting topological, mechanical and ECM (extracellular matrix) factors relevant to that test cell/cells) (i.e., test niche factors) and evaluating their ability to maintain in vitro, selected phenotype of the test cell/cells, using characteristics such as cell morphology and phenotype marker expression, as the first line screening for individual cell niche factors. The ability of the individual niche factor to maintain a selected characteristic of the test cell is determined by culturing the test cell/test cells on a substrate incorporating the individual niche factor and determining endpoints such as cell morphology and phenotype marker expression specific to the test cell/test cells.

In the second stage, using the phenotype-maintaining cell niche factors shortlisted from the first stage of screening (for example 1 mechanical, 2 topological and 2 ECM factors), protein microstructures integrating the shortlisted cell niche factors in combination are reconstituted and used to verify the optimal phenotype maintenance of the test cell/cells using endpoints such as cell morphology and phenotype marker expression specific to the test cell/test cells.

Also disclosed herein is a Multiphoton Microfabrication and Micropatterning (MMM) technology able to fabricate soluble protein/cell-cell adhesion molecule microstructures and micropatterns with controllable features on substrates such as a glass substrate. The MMM technology is an emerging micropatterning technique which is: (1) free from sophisticated glass surface and photomask preparation; (2) biocompatible without toxic reagents involved; (3) advantageous for superior lateral resolution (≈200 nm) resulted from two-photon adsorption as compared with commonly used UV light; (4) compatible with both 2D and 3D modalities.

Also disclosed herein are mask-free, non-contact and biocompatible multiphoton microfabrication and micropatterning methods involving microfabricating a neutral protein, e.g. serum albumin, preferably human or bovine serum albumin micro-structure on a substrate surface; micropatterning a layer of one member of an affinity pair such as avidin (in particular neutravidin) on the human or bovine serum albumin micro-structure; linking a soluble protein to the second member of an affinity pair, for example, when the first member is neutravidin, biotinylating a soluble cellular protein; and micropatterning the biotinylated soluble cellular protein through functional binding onto the micropatterned layer of neutravidin. It would be appreciated that other linker material for conjugating/interacting with its specific binding partner can be used in this invention. The linker material and its specific binding partner can be any pair of molecules with high affinity, specificity and antigen-antibody type of interactions and binding.

Also disclosed herein are associated biochips including the designs, the components, the materials, the usage, the preparation and associated analysis tools.

Multiplexed biochips associated with a combination of optimal cell niche factors such as those that mimic the vivo microenvironment of the cells in native tissues, either a single cell or a population of cells are disclosed

Also disclosed herein are the applications of the cell niche engineering platform and the biochips.

The cell niche generated are multiplexed and ready to be used directly because of the use of biocompatible materials especially the natural occurring protein and no harsh reagent and conditions used.

Applications of the disclosed methods and biochips disclosed herein includes reconstituting biomimetic cell niche to tailor-made cell culture substrates for physiologically relevant cell cultures and drug screening; optimizing biomimetic scaffold design for tissue engineering applications; micropatterning heterogeneous soluble niche factors for promoting signaling activities; and engineering asymmetric cell niche factors for manipulating asymmetric cell division (ACD) orientation and hence fates of the daughter cells of a single stem cell.

As exemplified herein using mouse embryonic stem cells, 3D micro-niches with protein micropillars functionalized with a selected ECM, fibronectin (FN), and a selected cell-cell interaction molecule, E-Cadherin (E-Cad), were fabricated to demonstrate the ability to incorporate biophysical and biochemical signals, with spatial control and retained bioactivities, into the immediate microenvironment of single cells.

### BRIEF DESCRIPTION OF THE DRAWINGS

FIG. 1A is schematic diagram illustrating the overall experimental design of the MMM-based multiplex cell niche screening for bNPC phenotype maintenance as an example, using a two-stage screening phase covering microfabrication of protein culture substrates with multiple cell niche factors. Top panel: (1) first-line screening on individual cell niche factors (2 mechanical properties; 9 topological features and 4 types of ECM niches at different magnitude) by culturing bNPCs on these culture substrates for 7 days, followed by phenotype evaluation to determine the shortlisted cell niche factors; and bottom panel: (2) second-line screening where bNPCs were cultured on microfabricated protein culture substrates with combinations of the shortlisted cell niche factors for 7 days, followed by phenotype evaluation to determine the optimal combination of the cell niche factors for bNPC phenotype maintenance. FIG. 1B depicts a schematic diagram of the set-up of multiphoton microfabrication and micropatterning technology in complex cell niche engineering with multiple niche factors, controllable dose and spatial arrangement by an iterative approach. In the left bottom diagram, protein solution such as bovine serum albumin (BSA), mixed with photosensitizer, on a suitable substratum such as glass in a biochip is placed on a xyz-controllable stage of a fabrication machine or a confocal microscope. Femto-second laser at a wavelength such as 800 nanometer is controlled to excite specific spots in the solution. Making use of the arbitrary spatial and intensity control of the laser spots together with the stage, microstructures with arbitrary topological architecture and mechanical strength are generated on the glass substratum of the biochip. In the diagram on the right bottom, functionalization of the structure with bioactive molecules such as extracellular matrix is performed similarly by an iterative approach. Bioactive molecules mixed with photosensitizer are crosslinked onto the structure surface without altering the mechanical properties with chosen doses by the spatial and intensity control of the laser. FIG. 1C depicts a schematic diagram illustrating the multi-stage screening system, specifically, the phenotype maintenance of a specific cell type, with the initial preparation phase. Left panel: Preparatory phase with isolation of cell preparation and determination of the screening parameters (such as cell, duration and phenotype marker readouts) via comparison between 3D native tissue and 2D monolayer cultures; Right panel: Two-stage screening phase covering microfabrication of protein culture substrates with multiple cell niche factors, not shown in the schematics; (1) first line screening on individual cell niche factors (mechanical properties, topological features and ECM niches at different magnitude) by culturing the cells on these culture substrates for 7 days, followed by phenotype evaluation to determine the shortlisted cell niche factors; and (2) second line screening where cells were cultured on microfabricated protein culture substrates with combinations of the shortlisted cell niche factors for a period of time, followed by phenotype evaluation to determine the optimal combination of the cell niche factors for phenotype maintenance. Additional stages for optimizing and fine-tuning the complex niche can be done with the result of the second line screening.

FIG. 1D depicts a generalized schematic of a biochip, with a top view and a perspective view. The biochips are glass chip with various types of cell niche designs on it. For the ease of handling, the chips are compartmentalized into microwells by 2 layers of polymer isolators with certain designs. Microstructures are located in the microwells and are ready for cell seeding and experiments. Different sets of niche conditions are organized in different microwells. FIG. 1E depicts an accessory case to facilitate the transplantation of biochips to the customers and the manipulation of the biochips during screening experiments. It includes a case body, a gate and a lid, in order to protect the biochip and prevent leakage of antibiotics and antimycotics solution during delivery. The materials used for the case body and the gate can be sterilized by autoclave and reused during the experiment and culture.

FIG. 2A-C show changes in phenotype of bovine nucleus pulposus cells (bNPCs) upon monolayer cultures. FIG. 2A bright-field images with phase contrast of bNPCs at day 4, 7, 10, 14. scale bars: 50 μm; FIG. 2B shows growth kinetics of primary bNPCs; y axis is in logarithm of the cell count normalized to initial cell number; proliferation of bNPCs substantially increased non-linearly; the doubling time of the cells was less than 14 h after 5 days; FIG. 2C shows Western blot of the phenotype marker proteins from total cell lysate prepared from bNPCs upon monolayer cultures for 4, 7, 10 and 14 days. The images were semi-quantified by the intensity of the protein bands and summarized in the line plot. The result showed that the expressions of selected marker proteins have significantly dropped within the first 7 days of culture. Phenotype marker expressions on day 4 were statistically significantly higher than later time points (day 7, 11 & 14) for all three protein markers (COL2, KRT8, SNAP25) at p<0.005, by One-way ANOVA with Bonferroni's post-hoc test. All experiments were conducted with at least 3 independent trials.

FIG. 3A-B. Ultrastructural characterization of the selected topological cell niche factors via SEM. (FIG. 3A) SEM images of the control group: flat BSA matrix (Flat Matrix; FM). Scale bar: 20 μm (i); 2 μm (ii) and (iii) 500 nm. (FIG. 3B) SEM images of the topological cues for screening. Abbreviation: (i) Micro-Pillar array (MPA); (ii) fiber-bead hierarchy (FB); (iii) thick grating (TkG); (iv) thin grating (TnG); (v) parallel grating hierarchy (GHpl); (vi) perpendicular grating hierarchy (GHpp); (vii) convex (Cv); and (viii) concave (Cc). Scale bars: 20 FIG. 3C shows fabrication parameters for mechanical niche factors. FIG. 3D shows schematic and set-up of the fabrication for various mechanical and topological factors. FIG. 3E shows the quantitative measurement (mean with 95% CI) of the staining intensity of the phenotype markers upon culturing of bNPCs on protein cell niche with different elastic modulus. FIG. 3F shows clustered bar chart showing the quantitative measurement (mean with 95% CI) of the staining intensity of the phenotype markers upon culturing of bNPCs on protein cell niche with different stiffness. Y-axis of bar charts: quantitative measurement of the IF staining intensity; Statistical significance: *p<0.05; **p<0.01, by one-way ANOVA with Bonferroni's post-hoc test. All groups at each level of niche properties and staining have a sample size of >100 cells from at least 3 independent experiments.

FIG. 4A is a clustered bar chart showing the quantitative measurement (mean with 95% CI) of the staining intensity of the phenotype markers upon culturing of bNPCs on protein cell niche with different topological features. FIG. 4B-C are bar charts showing the quantitative measurement (mean with 95% CI) of the roundness and aspect ratio of bNPCs cultured on protein cell niche with different topological features. Statistical significance: *p<0.05; **p<0.01, by one-way ANOVA with Tukey's post-hoc test. All groups at each level of niche properties and staining have a sample size of >100 cells from at least 3 independent experiments.

FIG. 5A-F show first line of screening of the extracellular matrix cell niche factors. FIG. 5A. Fabrication parameters. FIG. 5B. Schematic and set-up of the fabrication. FIG. 5C-F show clustered bar chart showing the quantitative measurement (mean with 95% CI) of the staining intensity of the phenotype markers upon culturing of bNPCs on protein cell niche with different ECM functionalization including (FIG. 5C) fibrinogen (Fg), (FIG. 5D) fibronectin (Fn), (FIG. 5E) laminin (Lm) & (FIG. 5F) vitronectin (Vtn). Y-axis of bar charts: quantitative measurement of the IF staining intensity; Statistical significance: *p<0.05; **p<0.01, by one-way ANOVA with Bonferroni's post-hoc test. All groups at each level of niche properties and staining have a sample size of >100 cells from at least 3 independent experiments.

FIG. 6A-B show the second line screening of the combinatory cell niches. (FIG. 6A) Schematic and set-up of the fabrication. FIG. 6B is a clustered bar chart showing the quantitative measurement (mean with 95% CI) of the staining intensity of the phenotype markers upon culturing of bNPCs on protein cell niche with different niche combinations. Y-axis of bar charts: quantitative measurement of the IF staining intensity; Statistical significance: *p<0.05; **p<0.01, by one-way ANOVA with Bonferroni's post-hoc test. All groups at each level of niche conditions and staining have a sample size of >100 cells from at least 3 independent experiments.

FIG. 7A-C depicts a schematic diagram showing the overall experimental design of the multiphoton-based micropatterning of BMP-2. FIG. 7A shows the process of Neutravidin (NA) micropattern fabrication and characterization. FIG. 7B. Biotinylation of rh-BMP-2 and characterization of biotinylated BMP-2. FIG. 7C shows the investigation on BMP signaling as evaluation of the bioactivity of the micropatterned BMP-2.

FIG. 8A-E depicts the spatially and quantitatively controlled micropatterning of functional neutravidin (NA) by multiphoton fabrication. FIG. 8A is a flow chart of the process of NA micropatterning and characterization. FIG. 8B shows the top and orthogonal view of fluorescent NA square micro-matrix written on the surface of BSA substrate after incubation with Atto 655-Biotin. Scale bar, 10 μm. FIG. 8C shows the z-axis profile of the relative mean fluorescence intensity of NA square micro-matrix fabricated at laser power of 45 mW and 3, 7 and 11 scan cycles after incubation with Atto 655-Biotin. The peak intensity is centered at 0 μm and the fluorescence intensity profile is fitted by Gaussian non-linear curve fitting (FIG. 8C). FIG. 8D, Quantitative analysis on the controllability of fabrication parameters over the local density of NA square micro-matrix. Linear regression in (I)-(V) showing the controllability of scan cycle over the local density of NA (peak fluorescence intensity) when keeping laser power and NA concentration constant; Linear regression in (VI) demonstrating the controllability of NA concentration over the NA crosslinking efficiency (the slope of linear curve in (I)-(V)) when keeping laser power constant; Non-linear regression in (VII) illustrating the controllability of laser power over the NA concentration-dependent crosslinking efficiency (the slope of linear curve in (VI)). n=6 in two independent experiments, error bars in (VI) and (VII) are SEM. FIG. 8E, Linear regression in (I) showing the controllability of scan cycle over the amount of Atto-Biotin bound on NA square micro-matrix; Non-linear regression in (II) demonstrating the controllability of laser power over the sensitivity of the NA-biotin binding (the slope of linear curve in (I)). n=2 in two independent experiments, error bars in (II) are SEM.

FIG. 9A-C depicts spatially and quantitatively controlled micropatterning of BMP-2 on NA micropatterns via NA-biotin interactions. FIG. 9A is a flow chart of biotinylating BMP-2 and characterization (I). Measurement of level of biotin labeling varied by different molar ratio of NHS-PEG4-Biotin to BMP-2 applied in the biotinylation process, n=4 in two independent experiments (II). Verification of NA-biotinylated BMP-2 interaction via indirect ELISA performed on the commercial NA-coated microplate, n=4 in two independent experiments (III). FIG. 9B shows a bioactivity assay of biotinylated BMP-2 (bBMP-2) with LOL of 4 or unlabeled BMP-2 (fBMP-2) applied in the cell culture medium at ascending concentrations (0, 100, 200 and 1000 ng ml⁻¹) to trigger pSmad 1/5/8 nuclear translocation in mouse myoblasts (C2C12 cell line) (denoted by white arrows). n=60 in two independent experiments at each concentration, error bars are SD. FIG. 9C shows a flow chart of micropatterning BMP-2 via NA-biotin binding process. FIG. 9D-E shows measurement of quantity of NA-bound BMP-2 in the form of measured BMP-2 immunofluorescence intensity. Non-linear regressions (FIG. 9D) showing the controllability of laser factor (scan cycle) and reagent factor (bBMP-2 applied for NA binding) over the quantity of NA-bound bBMP-2 whereas there is no such controllability over the quantity of NA-bound fBMP-2 (FIG. 9E). n=4 in two independent experiments.

FIG. 10A-E depicts sustained and higher level of Smad signaling triggered by micropatterned BMP-2. FIG. 10A-D, Quantitative analysis on the temporal change of nuclear accumulation of pSmad 1/5/8 (N/C ratio) upon BMP-2 treatment (fBMP-2 and bBMP-2 with different LOL of 2, 4 and 12) (4 hours (FIG. 10A); 24 hours (FIG. 10B); 48 hours (FIG. 10C); 72 hours (FIG. 10D)). Line plots showing the effects of BMP-2 treatment factor and time factor on N/C ratio analyzed by Two-way ANOVA (FIG. 10E). The means of N/C ratios among different groups are compared by One-way ANOVA with Bonferroni's post-hoc tests. n=176, 151, 145, 164 and 159 in two independent experiments for medium only, fBMP-2, bBMP-2 (LOL=2), bBMP-2 (LOL=4) and bBMP-2 (LOL=12) group, respectively, at 4 hours; n=133, 140, 160, 145 and 107 in two independent experiments for the above groups, respectively, at 24 hours; n=148, 171 and 172 in two independent experiments for medium only, fBMP-2 and bBMP-2 (LOL=4) group, respectively, at 48 hours; n=158, 172 and 179 in two independent experiments for the above groups, respectively, at 72 hours. Error bars in (A-D) and (E) are SD and 95% CI, respectively.

FIG. 11A-B depicts spatially and quantitatively controlled Smad signaling by micropatterned BMP-2. FIG. 11A, The dose-dependent nuclear accumulation of pSmad 1/5/8 (N/C ratio) controlled by BMP-2 ligand factor. Scatter dot plots showing the N/C ratio in C2C12 cells cultured on NA micropatterns incubated with ascending amounts (0, 125, 250, 500, 750 and 1000 ng) of bBMP-2 (LOL=2) (I), or bBMP-2 (LOL=4) (II), or on NA micropatterns with ascending concentrations (0, 125, 250, 500, 750 and 1000 ng ml⁻¹) of fBMP-2 in the culture medium (IV). Line plots showing the effects of bBMP-2 concentration factor and LOL factor (III), and fBMP-2 concentration factor (V) on N/C ratio dose-dependency. n=70, 83, 75, 84, 89 and 61 in two independent experiments in groups of 0, 125, 250, 500, 750 and 1000 ng of bBMP-2 (LOL=2), respectively; n=70, 83, 163, 100, 88 and 103 in two independent experiments in groups of the above amount of bBMP-2 (LOL=4), respectively; n=57, 60, 61, 57, 60 and 60 in two independent experiments in groups of 0, 125, 250, 500, 750 and 1000 ng ml⁻¹ of fBMP-2, respectively. Error bars in (I), (II) and (IV) are SD, error bars in (III) and (V) are 95% CI. FIG. 11B, The dose-dependent nuclear accumulation of pSmad 1/5/8 (N/C ratio) controlled by NA fabrication factor (biotin-binding capacity of NA). Scatter dot plots showing the N/C ratio in C2C12 cells cultured on NA micropatterns fabricated with the fixed laser power (36 mW) but ascending numbers of scan cycle (7, 9, 11, 13 and 15) followed by applying 270 ng of bBMP-2 (LOL=4) for NA binding. n=42, 48, 39, 57 and 68 in two independent experiments in groups of biotin-binding capacity of 82.6, 93.0, 106.1, 121.3 and 133.7 pmol/mm², respectively. Error bars are SD.

FIG. 12A-12C is a schematic diagram showing the overall experimental design. (FIG. 12A) The multiphoton microfabrication and micropatterning (MMM) technology-based 3D single cell micro-niche; (FIG. 12B) Biophysical signals in the micro-niche dominantly affected nucleus deformation and cell division direction, supported with both experimental and mathematical modeling results; (FIG. 12C) Asymmetric biochemical signals in the micro-niche dominantly affected the ACD orientation and hence the mESC polarity. (FIG. 12D) 6, 18 and 35K magnification SEM images of micropillars in micro-niche. (FIG. 12E) Structure stability test of BSA micro-niche through measuring the size of pillar and wall thickness from DO, D12, D24, D36, D48 to D60.

FIG. 13A-E. 3D Single cell micro-niche fabrication and biofunctionalization. (FIG. 13A) 1) Schematic of protein A/G crosslinking on flat micromatrix arrays. Peak fluorescence intensity of immobilized fluorescence secondary antibody was used for representing crosslinking density under different fabrication conditions. 2-6) Clustered bar charts of the dose-dependence of the fluorescence intensity of the immobilized fluorescence secondary antibody on the laser parameters (scan power including 0, 18, 33, 48, 63 mW and scan cycles including 0, 1, 5, 10) and the concentration of the fluorescence secondary antibody including 0, 0.56, 1.13, 2.25 and 4.5 mg/mL. (FIG. 13B) 1) Schematic of E-Cad-Fc immobilization on protein A/G crosslinked BSA micromatrix. Peak fluorescence intensity of Dylight 633 tagged E-Cad-Fc was used for representing immobilizing density of E-Cad-Fc under different fabrication conditions. 2-6) Clustered bar charts of the dose-dependence of the fluorescence intensity of the immobilized E-Cad-Fc on the laser parameters (scan power including 0, 18, 33, 48, 63 mW and scan cycles including 0, 1, 5, 10) and the concentration of the fluorescence tagged E-Cad-Fc including 0, 25, 50, 100 and 200 μg/mL. (FIG. 13C) MCF-7 attachment on E-Cad-Fc gradient surface. 1) Cartoon indicated E-Cad-Fc gradient distribution on BSA micromatrix. 2) Confocal fluorescence image of MCF-7. BSA micromatrix was red and cells were stained for β-catenin (green), E-Cad (magenta), DAPI (blue). Scale bar, 20 μm. 3) Number of cells attached on E-Cad-Fc gradient surface. Data were shown as Mean±SD. (N=793) (FIG. 13D) MCF-7 on E-Cad/FN alternative stripes surface. 1) Cartoon indicated E-Cad and FN distribution on BSA micromatrix. 2) Confocal fluorescence image of MCF-7 with XY, XZ and YZ section image. In XY image, the white dotted line indicated the region of E-Cad and FN stripes. In the XZ and YZ images, the white dotted line indicated the cell bottom of MFC-7. BSA micromatrix was red and cells were stained for β-catenin (green) and DAPI (blue). Scale bar, 20 μm. 3) Normalized fluorescence intensity of β-catenin on FN stripe and E-Cad-Fc stripe. Data were shown as Mean±SD and analyzed by two-sample t-Test (****p<0.0001). N=100 (FIG. 13E) SEM images of 3D micro-niche and mESC. Top view of 1) a complete 3D micro-niche and 2,3) two halves of 3D micro-niches. Side view of 4) a complete 3D micro-niche and 5,6) two halves of 3D micro-niches. Top view of 7) single mESC (green) in a 3D micro-niche (light brown) and 9) two cells after cell division (green) in a 3D micro-niche (light brown).

FIG. 14A-I. Manipulating nucleus deformation alignment and cell division direction in 3D micro-niche through controlling its 3D geometry. (FIG. 14A) Schematic diagram showing the design of the 3D micro-niche and the subsequent mESC cell culture and measurement for nucleus deformation alignment and cell division direction. (FIG. 14B) Diagram of mESC cell adhered on two, four and six wall micropillars and expected tensile stress force generation. (FIG. 14C) SEM images of 2, 4 and 6 micropillars micro-niche. 1-3) Top view with complete micro-niche (first row) and 4-6) side view with half micro-niche (second row). (FIG. 14D-E) Wall micropillars were crosslinked and functionalized with FN/FN (first row), E-Cad/E-Cad (second row) and FN/E-Cad (third row) on the opposite position. In the representative confocal image, 3D micro-niche was red and cell nucleus was blue. (FIG. 14D) Distribution of mESC nucleus deformation alignment (alignment of the long axis of nucleus white dotted circles) before cell division upon binding to biofunctionalized 2, 4 and 6 micropillars micro-niche. (FIG. 14E) Distribution of mESC at different cell division direction (axis connecting the center of mass of the two divided cell's nucleus white dotted lines) angles in biofunctionalized 2, 4 and 6 micropillars micro-niche. (FIG. 14F) Diagram of mESC cell adhered on tilted wall micropillars and expected tensile stress force generation. (FIG. 14G) SEM images of tilted wall micropillars micro-niche from 1) top view with complete micro-niche and 2) side view with half micro-niche. (FIG. 14H) Distribution of cell division direction (axis connecting the center of mass of the two divided cell's nucleus white dotted lines) in biofunctionalized tilted wall micropillars micro-niche. (FIG. 14I) Confocal image of 2 micropillars with different z-axis height in 3D micro-niche. The angle 9 between 2 micropillars were measured through connecting the center of surface, shown as the white dotted line. Scale bar, 5 μm. (FIG. 14J) Measurement of angles in biofunctionalized 2 micropillars 3D micro-niche. Blue line showed the fitted normal distribution of angle.

FIG. 15A-F. Mathematical model that explains cell division direction in 3D micro-niche. (FIG. 15A) Schematic of a model cell and the relative position of cell membrane adhesion protein, cortex binding molecule, astral microtubule, cortical actin, spindle pole and cell nuclear inside the cell. (FIG. 15B) Schematic of force transduction from outside-in. (FIG. 15C-F) Modeling results of cell alignment and cell division direction in 2, 4, 6, and 2 with different z-axis height micropillars micro-niche, respectively. 1) The schematic diagram illustrating the cell in 3D micro-niche; 2) The noncompensated force F(α) generated by microtubules (green); 3) The probability density P(α) of cell division direction (blue); 4) The torque force T(α) experienced by cell nucleus (red). (FIG. 15G) Position of cell nucleus center relative to 3D micro-niche center are not significantly different, *p>0.05. (not used). (FIG. 15H). Measurement of z-axis angles in 2, 4, 6 micropillars 3D micro-niche. No significant difference was found using one way anova analysis. N=39, 43, 40 for 2, 4, 6 micropillars 3D micro-niche, respectively. (FIG. 15I) Measurement of mESC cell nucleus radius. N=42. (FIG. 15J) Measurement of distance between corresponding micropillars. N=228.

FIG. 16A-G. Cell division direction in 3D micro-niche was determined by the tensile stress force but not the cell shape. (FIG. 16A) 1-3) Diagram of cell resided in 6, 4 and 2 FN-functionalized micropillar micro-niche and expected tensile stress force generation. 4-6) SEM images of 6, 4 and 2 FN-functionalized micropillar micro-niche. (FIG. 16B) Quantification of cell area and roundness using confocal with phase contrast images in 6, 4 and 2 FN-functionalized micropillar micro-niche. (FIG. 16C) 1) Distribution of mESC at different cell division direction (axis connecting the center of mass of the two divided cells' nucleus) angles in 6 FN-functionalized micropillar micro-niche. In the representative confocal image, 3D micro-niche was red and cell nucleus was blue. Scale bar, 5 μm. 2-4) Modeling results of the noncompensated force F(α) (green), the probability density P(α) of cell division direction (blue), and the torque force T(α) experienced by cell nucleus (red). (FIG. 16D) 1) Distribution of mESC at different cell division direction (axis connecting the center of mass of the two divided cells' nucleus) angles in 4 FN-functionalized micropillar micro-niche. In the representative confocal image, 3D micro-niche was red and cell nucleus was blue. 2-4) Modeling results of the noncompensated force F(α) (green), the probability density P(α) of cell division direction (blue), and the torque force T(α) experienced by cell nucleus (red). (FIG. 16E) 1) Distribution of mESC at different cell division direction (axis connecting the center of mass of the two divided cells' nucleus) angles in 2 FN-functionalized micropillar micro-niche. In the representative confocal image, 3D micro-niche was red and cell nucleus was blue. 2-4) Modeling results of the noncompensated force F(α) (green), the probability density P(α) of cell division direction (blue), and the torque force T(α) experienced by cell nucleus (red). (FIG. 16F) 1-3) SEM images from side view of 6, 4 and 2 FN-functionalized micropillar micro-niche. (FIG. 16G) Quantification of cell aspect ratio (1), circularity (2) and solidity (3) using confocal with phase contrast images in 6, 4 and 2 FN-functionalized micropillar micro-niche.

FIG. 17. Cell polarity formation and ACD control in 3D micro-niche. (FIG. 17A) Two pillar 3D micro-niche. 1-3) Schematic diagrams of two pillar 3D asymmetric micro-niche with FN/E-Cad, FN/FN, and E-CAD/E-Cad. 4-6) SEM images of two pillar 3D asymmetric micro-niche with FN/E-Cad, FN/FN, and E-CAD/E-Cad. 7-9) Confocal images of two pillar 3D asymmetric micro-niche with FN/E-Cad, FN/FN, and E-CAD/E-Cad. FN was green and E-Cad was magenta. Scale bar, 5 μm. (FIG. 17B) Fluorescence signal distribution plot of pFAK and active integrin β1 (1), aPKC and pan-cadherin (2), LGN and YAP (3), Nanog and SSEA1 (4) inside mESC. (FIG. 17C) Quantification of ACD orientation inside asymmetric micro-niche FN/E-Cad. Chi square analysis was performed, significance level was set as 0.0005. (FIG. 17D) Fluorescence signal distribution plot of pFAK and active integrin (31 (1), aPKC and pan-cadherin (2), LGN and YAP (3), Nanog and SSEA1 (4) inside mESC. (FIG. 17E) Quantification of ACD orientation inside symmetric micro-niche FN/FN. Chi square analysis was performed, significance level was set as 0.0005. (FIG. 17F) Fluorescence signal distribution plot of pFAK and active integrin (31 (1), aPKC and pan-cadherin (2), LGN and YAP (3), Nanog and SSEA1 (4) inside mESC. (FIG. 17G) Quantification of ACD orientation inside symmetric micro-niche E-Cad/E-Cad. Chi square analysis was performed, significance level was set as 0.0005. (FIG. 17H) Logarithm base 10 of FIR of Nanog in asymmetric FN/E-Cad, and symmetric FN/FN, E-Cad/E-Cad micro-niches. One way ANOVA was performed, significance level was set as 0.05. (FIG. 17I) Logarithm base 10 of FIR of SSEA1 in asymmetric FN/E-Cad, and symmetric FN/FN, E-Cad/E-Cad micro-niches. One way ANOVA was performed, significance level was set as 0.05.

FIG. 18. Working model for asymmetric micro-niche induced cell polarity and oriented ACD.

### DETAILED DESCRIPTION OF THE INVENTION

Cell niche represents the highly heterogeneous microenvironment that maintains, regulates and manipulates the resident cells for important cellular fate processes including but are not limited to phenotype maintenance, proliferation, lineage guidance or even apoptosis. However, when cells are isolated from tissues for in vitro culture in research, biotechnology and pharmaceutical industries, they no longer reside in their native cell microenvironment and hence rapidly lose their physiological morphology, phenotype, properties and functions, adversely affecting the physiological relevance of the cellular behavior observed during cell culture and their responses to extrinsic agents during drug and toxicity screening. Identifying, designing and reconstituting the optimal complex cell niches is the key step to advance the current monolayer in-vitro culture system into a physiologically relevant system, and to assure the validity and reliability of cell culture-based drug screening and toxicity screening.

An ideal niche engineering technology should fulfill a number of criteria. It should readily incorporate various kinds of niche factors independently with sufficient sub-cellular spatial resolution in a highly controllable manner, as discussed in the background section. Multiphoton microfabrication and micropatterning (MMM) platform therefore supersedes the conventional approaches in this field of cell niche engineering. Furthermore, the high-throughput technology should neither be limited to the level of a single comparison experiment nor a single laboratory. Therefore, both conversion of the technology to screening studies and translation to products and services levels must be considered.

The biochip system disclosed herein overcomes several of these limitations with the multiphoton microfabrication and micropatterning platform as a more ideal niche engineering technology, together with the presentation of systematic screening approach for niche screening.

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### I. Multiplexed Biochips Incorporating Optimal Cell Niche Factors

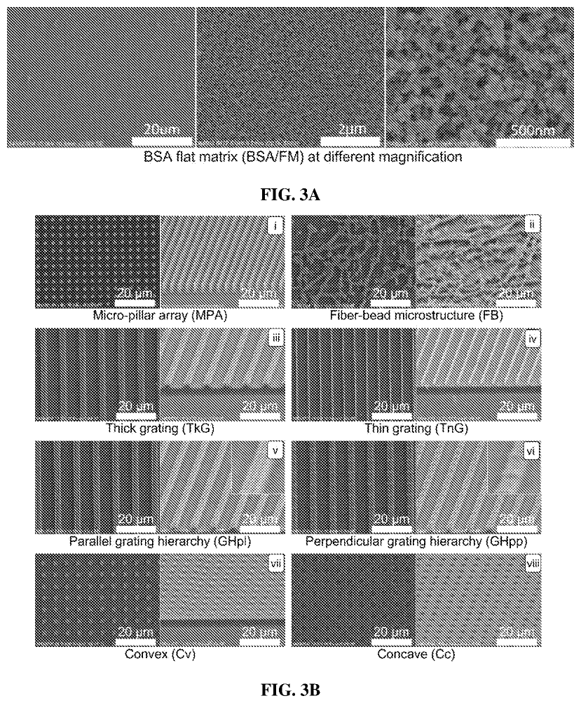

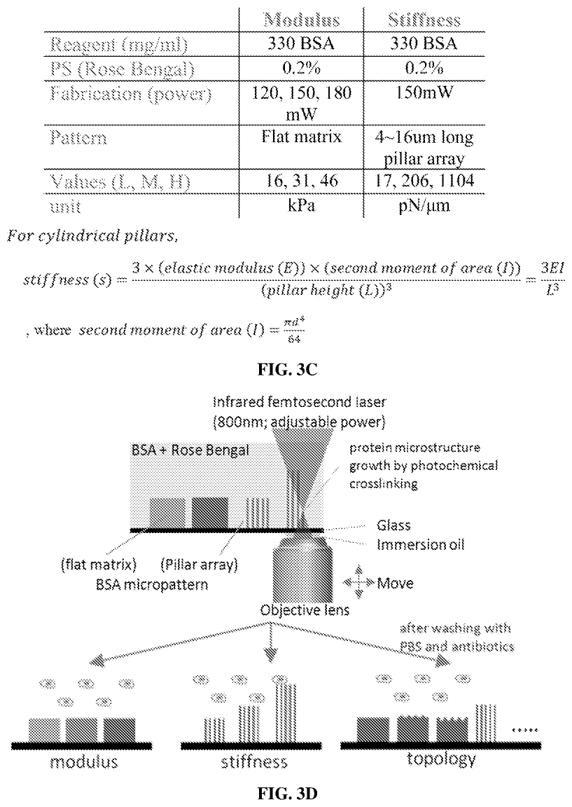

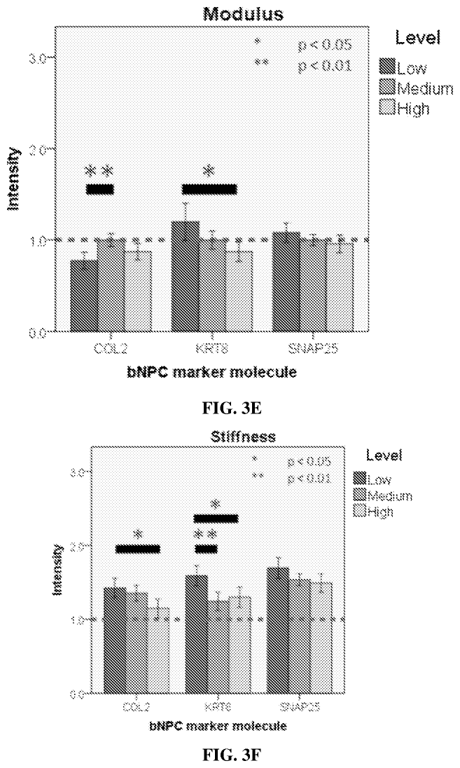

Organizing the multiple niche designs into biochip formats is a critical step of this technology. Several formats of biochips are disclosed herein. Generally, the disclosed biochip in one embodiment includes a first and a second layer and incorporate one or more cell niche factors. Cell niche factors include, but are not limited to topological factors, mechanical factors, extracellular matrix factors, cell-cell adhesion molecules, and soluble proteins/bioactive factors. Exemplary topological factors include, but are not limited to factors such as flat matrix (BSA/FM) (FIG. 3A), micro-pillar array (MPA) (FIG. 3B(i)), fiber-bead microstructure (FB) (FIG. 3B(ii)), thick grating (TkG) (FIG. 3B(iii)), thin grating (TnG) (FIG. 3B(iv)), parallel grating hierarchy (GHpl) (FIG. 3b (v)), perpendicular grating hierarchy (GHpp) (FIG. 3B(vi)), convex (Cv) (FIG. 3B(vii)) and concave (Cc) (FIG. 3B(viii)). The topology selections can include regular contours or irregular contours (mix of different regular contours).

The first layer is a macrostructure which includes a solid support having a top surface, micropatterned as disclosed herein to include a plurality of microstructures which represent topological cell niche factors in a second layer and biological cell niche factors preferably attached thereto.

The second layer includes a biosupport and biological cell niche factors or any biofactor such as proteins including but are not limited to growth factors, cytokines and interleukins, other biomolecules such as lipids, carbohydates, nucleic acids (mRNA, siRNA, DNA etc), and other biomolecules such as extracellular matrix components including proteins, carbohydrates, glycoproteins, proteoglycans, lipids, which in combination, form the miscrostructures. The biological cell niche factors include extracellular matrix (ECM) macromolecule and soluble factors. The cell biochip can include symmetrical or asymmetrical biochemical niche factors, for example FN/FN or E-Cad/E-Cad or asymmetric for example FN/E-Cad (for example asymmetric biochemical niches, in particular, a matrix niche FN on a top micropillar, and a cell niche E-Cad on a bottom micropillar). Any combination of niche factors can be presented in a symmetrical or assymetrical fashion as exemplified herein for FN/E-Cad.

The solid support can be made from any suitable material used to make biochips, including, but not limited to glass, silicon, fused quartz, plastic etc. “Biosupport” as used herein refers to a supporting structure in the microstructure which is made from a molecule preferably a naturally occurring biomolecule including but are not limited to proteins, carbohydrates, lipids, proteoglycans, glycoproteins, nucleic acids. An exemplary biosupport protein is albumin, preferably human or bovine albumin, or any protein that would bind to the photosensitizer (for example, rose bengal), for example, an ECM molecule such as fibronectin, gelatin, laminin, histone, fibrinogen and collagen.

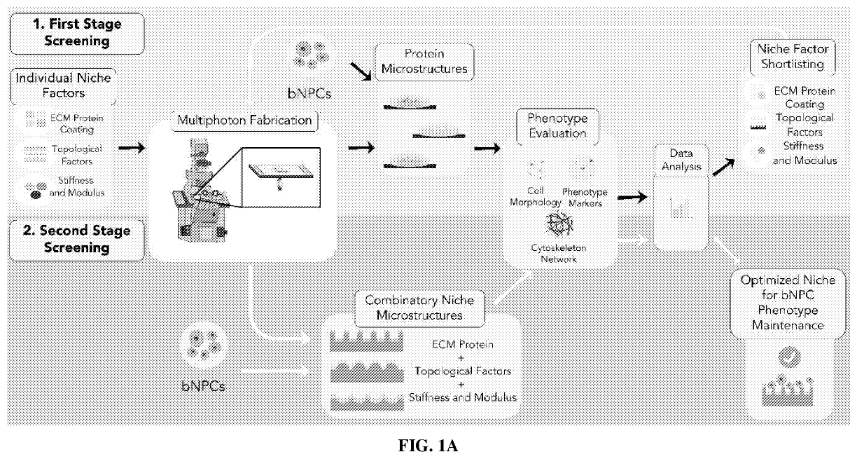

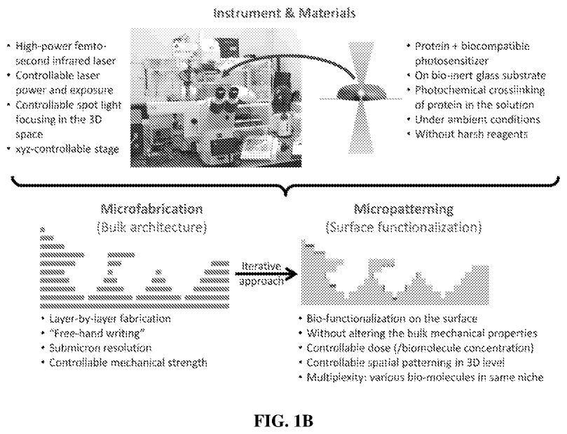

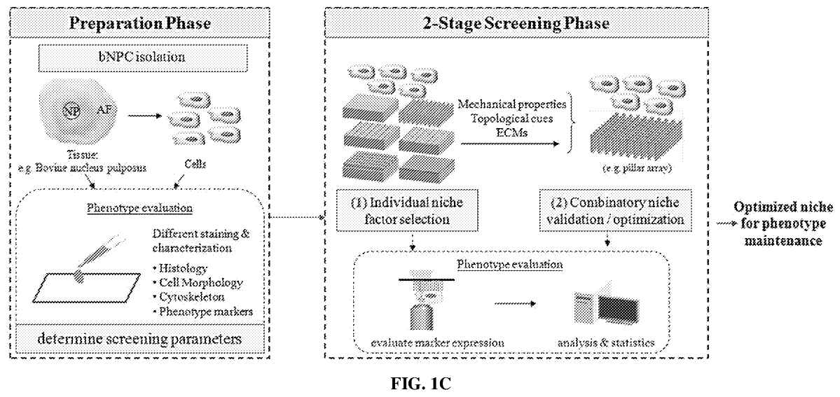

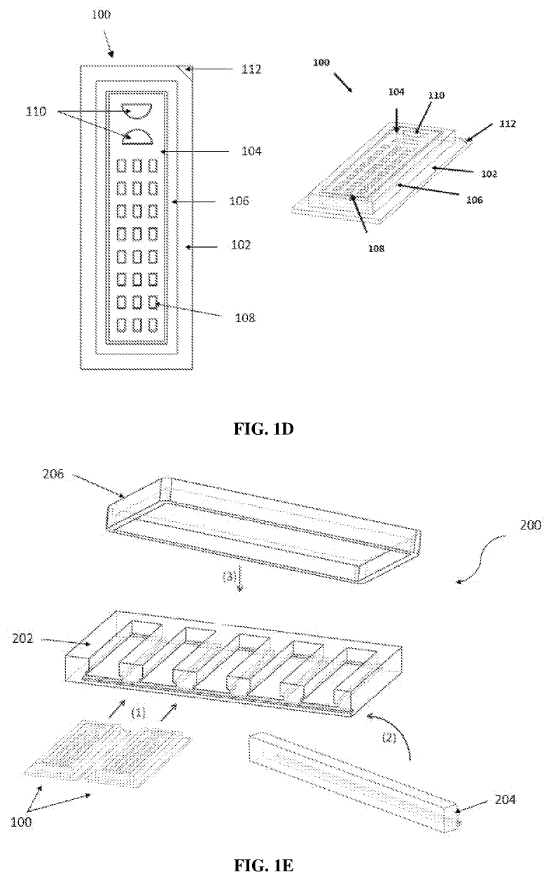

FIG. 1D shows the schematic design of components used to make the disclosed biochips 100, in one embodiment. The components include a flat solid support such as a glass support 102, a removable inner isolator 104 and a removable outer isolator 106. The removable inner isolator 104 is a grid-like structure, which includes openings (microwells) in shapes such as rectangular, cubic, cylindrical, etc., preferably uniform in size. The glass chip 102 is compartmentalized into microstructures using the microwells 108 provided by the inner isolator 104. The inner isolator 104 can contain a desired number of openings 108, provided by its gride-like nature, for example, 12, 18, 24, 40, 50 etc. The inner and outer isolators 104, 106 can be made from any suitable hydrophobic and elastic material such as plastic, silicone, polymer (e.g. polyisoprene), etc. The outer isolator 106 is configured to surround the circumference of the inner isolator 104. Thus, where the inner isolator 104 is rectangular in shape, the outer isolator 106 is a hollow rectangle, which when lowered onto the inner isolator 104, fits around the circumference of the inner isolator 104. The inner isolator 104 and the outer isolator 106 are provided for easy handling and experimental flexibility. The two notches 110 at the top part of the inner isolator 104 allows the consumers to easily detach it without the risk of damaging the microstructures deposited on the solid support 102. The outer isolator 106 also enables fast manipulation of medium or staining when the inner isolator 104 is detached. The top right corner 112 of the solid support 102 is preferably marked, to remind the user of the front and back sides of the biochip 100, particularly when both isolators 104, 106 are removed. The biochips may be delivered to the customer with the microwells immersed in antibiotics and antimycotics solution or in a dehydrated format.

In another embodiment, the biochip is provided in a microplate format which includes microwells. A microplate is used herein to refer to is a flat plate with multiple “wells” used as small test tube. A microplate typically has 6, 12, 24, 48, 96, 384 or 1536 sample wells arranged in a 2:3 rectangular matrix. Each well of a microplate typically holds somewhere between a few nanolitres to several millilitres of liquid. Some microplates have been manufactured with 3456 or 9600 wells. In this embodiment, microstructures containing cell niche factors are deposited onto the bottom of a solid support which includes microwells. This is in contrast to the first embodiment, which the grid structure of removable inner isolator provides the microwells used to deposit microstructures onto a flat solid support.

In either embodiment, microwells can be between about 0.5 to about 10 mm in height and the deposited microstructures can range in height from 1 μm to about 50 for example, from about 1 to about 10 μm.

FIG. 1E is a design of an accessory case for the glass biochip 100 for protection and easy manipulation. The case 200 consists of a main body 202, a gate 204 and a transparent polymer lid 206. It allows multiple glass biochips 100 to be manipulated readily inside the biosafety cabinets and incubators. It can also be used to transport the chip from the manufacturers to the users safely without the leakage of solutions.

In one embodiment, the disclosed biochips include one of more cell microniche factors shown in Table 1 or other biofactors such as carbohydrates, lipids and nucleic acids.

TABLE 1
Cell Niche Factors

| | Soluble | | | Cell-Cell |
|---|---|---|---|---|
| ECM Proteins | factors | Topological | Mechanical | interaction |
| Fibronectin | BMP2 | Micro-pillar | Reduced | E-cadherin |
| | | array | Elastic | |
| | | | modulus | |
| Collagen 4 | Wnt3a | Concave | stiffness | N-cadherin |
| Vitronectin | EGF | Convex | active force | |
| | | | cell niche | |
| | | | (4D printing) | |
| Fibrinogen | bFGF | Parallel Grating | | |
| | | Hierarchy | | |
| Laminin521 | TGF-β | Perpendicular | | |
| | | Grating | | |
| | | Hierarchy | | |
| Laminin mixture | BMP4 | Thick Grating | | |
| Collagen 1 | Wnt5a | Fiber-bead | | |
| | | microstructure | | |
| Laminin 411 | IL-2 | Wavy | | |
| Laminin 511 | IL-18 | Microwells | | |
| | | with/without | | |
| | | internal pillars | | |
| Collagen 2 | | Cave | | |
| Collagen 6 | | irregular | | |
| | | contours | | |
| Thrombospondin | | Villi | | |
| Tenacin | | | | |
| Mucin | | | | |
| Byglycan | | | | |
| Aggrecan | | | | |
| Decorin | | | | |

Topological features are shown in FIG. 3B(i)-(viii).

Preferably the biochip includes topological structures such as micro-pillar arrays and/or fiber beads, coated with ECM niche factors such as vitronectin, laminin, deposited on a suitable substrate such as glass by MMM, wherein the ECM proteins are crosslinked onto the topological structures.

This NPC study disclosed in the Example serves simply as an application example to the technology disclosed.

A. Soluble Biofactor Functionalized Biochip

One of the most challenging technological obstacles in constructing protein biochips is how to maintain the integrities, native conformations and bioactivities of the immobilized proteins on the chip surface as many microfabrication and micropatterning technologies involving physical or chemical crosslinking via covalent bonding denature proteins and hence compromise their integrity and bioactivities. Existing micropatterning techniques (e.g., microcontact printing, inkjet printing, stereolithography, electron beam) typically involve sophisticated processes, such as time-consuming surface functionalization, complicated preparation of photomask or polydimethylsiloxane (PDMS) stamp, and less biocompatible reagents. More importantly, simultaneously spatial- and quantity-controlled immobilization of either party of the host-guest binding pairs challenges the commonly used micropatterning modalities. As a result, a simple, effective and universal platform capable to micropattern host-guest binding pairs and hence to micropattern fragile proteins such as growth factors on biochips is warranted.

Methods disclosed herein provide biochips functionalized with a soluble protein/bioactive factors or an array of bioactive active factors/soluble proteins. First, gene microarrays: protein biochips comprising multiple soluble niche factors made by this disclosure, which are able to transduce multiple signaling pathways, can be developed as gene microarrays for profiling the landscape of gene expression in less-known cells growing on these chips. As such, this information could help biologist quickly screen out the most relevant signaling pathways for further investigation.

Second, soluble cell niche with counter-gradients of morphogens/antagonists: given the fact that important developmental processes, such as body pattern formation, are tightly regulated by counter-gradients of morphogen/morphogen or of morphogen/antagonist. According to this disclosure, a spatial- and quantity-controlled microfabrication and micropatterning technology, is able to reconstitute this counter-gradients of different morphogens with opposite biological effects or of morphogen and its antagonist, thus providing a useful in vitro tool for developmental biologist to interrogate the events related to morphogenesis that are not easily mimicked by other in vitro models such as monolayer cultures.

Third, solid-phased drug/biomolecule biochip: the surface-bound drugs or biomolecules with sustained and enhanced bioactivity fit for fabricating long-term bioactive culture chips, or functional scaffold chips, as well as drug carrier chips for screening out the optimized combination of components in scaffolds for tissue engineering or for new drug development in pharmaceutical industry.

The disclosed soluble factor biochips include a solid substrate, an inert protein layer, a micro pattern of a crosslinked first member of an affinity pair, deposited on the inert protein layer using a two photon fabrication system, and a bioactive protein/bioactive factor conjugated to a second member of an affinity pair, the bioactive factor/soluble protein linked to the inert protein layer via association of the affinity pairs. The bioactive factor/soluble protein can be any bioactive factor/soluble protein, including, but not limited to nucleic acids, growth factors (See also, Table 1), and the affinity binding cab be affinity pairs known in the art for example, be (e.g., extracellular matrix (ECM)-growth factor, albumin binding domain (ABD)-serum albumin (SA), barnase-barster), or those used as tools for protein purification in research or biotechnology (e.g., biotin-neutr/strept(avidin), Fc-protein A/G, His-nickel nitrilotriacetic acid (Ni-NTA)).

B. Single Cell 3D Micro-Niche Biochips

A single cell 3D micro-niche platform with controllable and engineerable local cell niche factors is provided, which finds applications in studies aimed at better understanding of the impact of local niche factors on mESC behavior and fate.

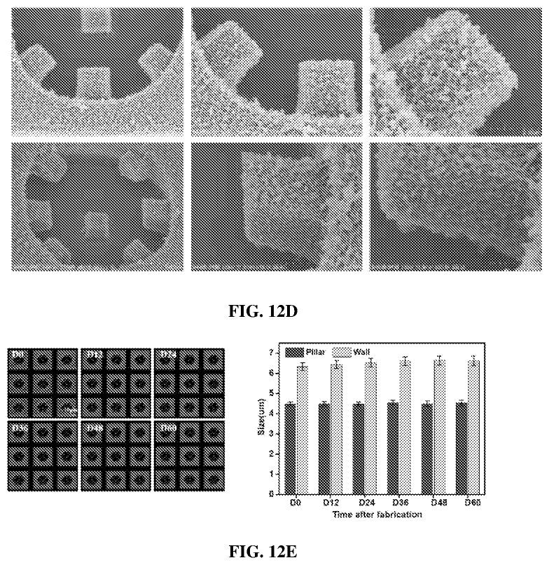

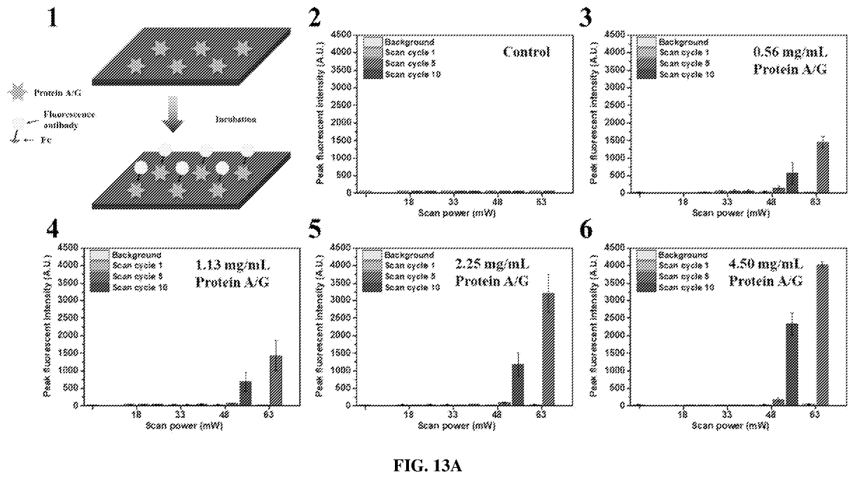

MMM technology is used to engineered a high throughput single cell-based 3D micro-niche with spatially controlled biophysical and biochemical signals (FIG. 12A-E). A stepwise process is used to fabricate thousands of 3D micro-niches (FIG. 12A) for high throughput and large scale single cell-based cell niche studies.

As exemplified herein using mouse embryonic stem cells, 3D micro-niches with protein micropillars functionalized with a selected ECM, fibronectin (FN), and a selected cell-cell interaction molecule, E-Cadherin (E-Cad), were fabricated to demonstrate the ability to incorporate biophysical and biochemical signals, with spatial control and retained bioactivities, into the immediate microenvironment of single cells. Mouse embryonic stem cell (mESC) was used to demonstrate the effects of engineered cell niche signals on their polarity and associated characteristics including nucleus deformation, cell division direction and ACD orientation. The Examples demonstrate a robust and high-throughput 3D micro-niche platform for single cell-based cell niche studies and demonstrates successful manipulation of cell division and ACD polarities via precisely and spatially engineered asymmetric biochemical niches. As schematic diagram showing multiphoton microfabrication and micropatterning (MMM) technology-based 3D single cell micro-niche is shown in FIG. 12A.

The 3D single micro-niche biochips include a solid substrate such as glass or silicon on which an inert protein such as BSA is deposited using MMM, and functionalized with a combination of factors specific for the local in vivo microenvironment (microniche) of a cell of interest (selected for the specific cell type using the two stage screening methods disclosed herein), for example, one or more ECM proteins, cell-cell attachment proteins such as E-Cadherin, one or more soluble proteins and one or more topological or mechanical factors (See table 1). The soluble proteins and cell-attachment proteins are attached to the 3D structure using the indirect affinity-binding pair strategy disclosed above for soluble proteins/bioactive factors. Collectively, a laser power at 48 mW, a laser scan cycle of 5, a protein A/G concentration of 4.5 mg/ml and a fluorescence tagged E-Cad-Fc concentration of 200 μg/ml were identified as the optimal specification for the indirect immobilization of the E-Cad-Fc on the micro-niche. A stepwise multiphoton-based microfabrication process is used to micro-print the wall, the bottom micropillars and the lateral wall micropillars, before using the same multiphoton-based micropatterning process to bio-functionalize the surfaces of the micropillars with a matrix niche protein(s) or cell-cell attachment proteins. The Examples demonstrate fabrication of 3D micro-niches with fixed dimension (28×28×20 μm) and identical topological features (6 micropillars evenly distributed with 60 degree between each other), while the 6 micropillars were selectively functionalized with a biochemical niche signal (matrix niche FN) in three differentially and spatially controlled patterns, all six micropillars, four diagonal micropillars, or the two pivotal micropillars.

Exemplary micro niche dimensions include: micro-niche outer wall length of about 25-45 μm, preferably between about 28 and 37 μm and the height of about 20 the inner aperture diameter of about 10-25 μm, preferably, about 15-20 μm. The distance between corresponding pillars can be about 8-15 μm, preferably between about 10.2-12 μm, and the z-axis distribution from about 10 to 13 μm from glass surface. The length of bottom pillar can be between 1-6 μm, preferably between about 3 to 4 μm and the height between about 1-5 μm, preferably about 3 μm. For the tilted pillar micro-niche, the micro-niche outer wall length can be about 37 μm, the inner aperture diameter about 20 μm, the wall height about 20 μm, and the z-axis distribution of two corresponding pillars from about 3 to 6 μm and from 10 to 13 μm.

Applications of the disclosed biochips include drug testing, cell specific interactions, etc. For example, biochips chips with specific niches for further investigation such as the cell response to drugs in a more physiologically or pathologically relevant niche environment. While the first one can be presented as a fixed product model, the latter two requires information support and communication with the customers. Potential products in the kit may also include the screening protocol and the data management software developed.

The disclosed and exemplified multiplex cell niche screening platform enables the design of an optimal cell niche biochips for in-vitro phenotype maintenance of cells, which are otherwise poorly maintained by the conventional 2D monolayer culture.

The disclosed biochips can be used to manipulate the cell fate of a cell or cells by culturing the cell(s) on the microstructures containing niche factors. For example, the single cell niche biochip disclosed herein engineered an asymmetric cell niche (FN on one side, Ecad on the other side, of the single cell microwell) and that manipulated the fate of the ESC by affecting the orientation of a key process called ACD (Asymmetric cell division). This is an example of manipulating cell fate through the engineered cell niche. The cells are cultured for an effective amount of time to bind the cells to the microstructures are cultured with an appropriate cell culture medium, selected based on the cell type being cultured, monitoring the cell fate of interest. Cell culture media are known and are commercially available. For example, Eagles medium, RPMI (Roswell Park Memorial institute: Gibco), DMEM (Dulbecco's Modified Eagle Medium), MEM (Minimum essential media), Neurobasal medium (21103049; Gibco), N2B27 basal medium (one volume of DMEM/F12 medium combined with one volume of Neurobasal medium supplemented with 0.5% N2 Supplement (17502048; Gibco), 1% B27 Supplement (17504001; Gibco). The cell culture media is supplemented as needed, for example, with BSA, penicillin and streptomycin, PD325901 (04-0006-10; Stemgent), CHIR99021 (04-0004-02; Stemgent), Glutamine (35050061; Gibco), β-mercaptoethanol (21985023; Gibco), Leukemia inhibitor factor (ESG1106; Sigma-Aldrich) and inactivated ES-FBS (10439016; Gibco). Cells are cultured on biochip microstructures in some embodiments as exemplified herein for embryonic stem cells.

In embodiments, the cells may be embryonic stem cells, mesenchymal stem cells or iPSC.

### II. Method of Making Soluble Biofactor Functionalized Biochips

One embodiment provides a multiphoton-induced, photo-chemical crosslinking-based fabrication and patterning technology at micron- or sub-micron scale, totally different from conventional microfluidics platforms. The multiphoton laser-based technology allows the fabrication of arbitrary shapes of microstructure in both 2D and 3D, superior to reconstitute the multiplex geometries in native microenvironment. Additionally, the magnitude of biomolecules such as neutravidin in this case, is tuned by either laser parameters (i.e., laser power or scan cycle) or reagent parameters (i.e., concentration of neutravidin in the fabrication solution) in the fabrication process rather than flow rates in microchannel arrays described in conventional methods.

Another embodiment of this disclosure presents a mask-free, laser free-form writing based technology to micropatterning avidin in particular neutravidin so as to micropattern biotinylated soluble factors. Neutravidin is directly micropatterned on the prefabricated BSA micro-substrate by two-photon induced photochemical crosslinking in register with the arbitrary ROI. There are no such time-consuming steps involved in the disclosed methods, such as glass surface modification, photomask fabrication, and prefabricated biotin micropatterns, which are essential in conventional methods. Moreover, the local density of soluble factor micropatterns can be precisely controlled by varying either the laser parameters or reagent parameters in the process of micropatterning neutravidin, which cannot be achieved in conventional methods. The Examples demonstrates that soluble factors functionalized onto a biochip using the disclosed methods, exemplified herein with t neutravidin-bound soluble factors, e.g., BMP-2, can exert a more sustained and a higher level of downstream signaling event, laying a foundation to engineering an artificial cell niche.

Making use of host-guest binding pairs with high affinity (also referred to herein as affinity binding pairs), for example, those naturally occurring in native biological system (e.g., extracellular matrix (ECM)-growth factor, albumin binding domain (ABD)-serum albumin (SA), barnase-barster), or those used as tools for protein purification in research or biotechnology (e.g., biotin-neutr/strept(avidin), Fc-protein A/G, His-nickel nitrilotriacetic acid (Ni-NTA)), are coupled with micropatterning technology, and this allowed spatial-controlled supermolecule interaction with a high specificity and directionality, and the flexible selection of type and number of host-guest binding pair to achieve micropatterning multiple types of protein in a quantitatively controllable manner.

Methods are disclosed to spatially and quantitatively micropattern bioactive molecules such as growth factors (e.g., bone morphogenetic protein 2 (BMP-2)) on the solid planer surface (e.g., glass surface) through immobilizing the host (e.g., neutravidin) of the host-guest binding pairs (e.g., neutravidin-biotin) on the pre-fabricated inert protein substrate (e.g., bovine serum albumin (BSA)) via two-photon induced photochemical crosslinking, followed by bathing with protein of interest tagged with specifically affinitive guest molecule (e.g., biotin). The multiphoton-based micropatterning technology together with the host-guest chemistry retained the bioactivities and triggered the downstream signaling pathways of soluble growth factors. Soluble growth factors immobilized and micropatterned in this manner show enhanced and sustained bioactivities, outperforming the free soluble growth factors supplemented to the culture medium. This multiphoton-based solid-phase biomolecule biochip facilitates basic research, new drug screening, scaffold design for tissue engineering and regenerative medicine, and beyond.

As described herein, the method (1) covalently micropatterns the host binding partner (one member of an affinity pair) on an inert protein substrate surface in a spatially and quantitatively controlled manner without disturbing their affinities for the guest binding partner; (2) non-covalently micropatterns the target proteins or drugs conjugated with the guest binding partner (i.e., the second member of an affinity pair) in concert with the host binding partner micropatterns in a spatially and quantitatively controlled manner; and (3) the micropatterned proteins or drugs potentiates and sustains their biological effects on the biochip. With multiple improved features and advantages, such as simplicity, integration of microfabrication and micropatterning in a single platform, biocompatibility, simultaneous spatial and quantitative controllability, and sustained and enhanced bioactivity of the immobilized protein, this disclosure holds a great potential to become an incubator leading to multiple future products, including, but not limited to the following three categories.

BMP-2 and neutravidin-biotin are used in in the Examples as an example of soluble factor and of host-guest binding pair are taken, respectively, to demonstrate that the use of MMM technology to micropattern a broad spectrum of soluble factors with sustained and enhanced bioactivities in a spatial- and quantity-controlled manner by utilizing various types of host-guest binding pair, i.e., the successful result demonstrated in micropatterning BMP-2 as shown in Example, while maintaining its biological activity can be readily extrapolated to other soluble bioactive factors/proteins other than BMP-2; and other types of host-guest binding/affinity pairs. Exemplary bioactive/soluble factors include, but are not limited to Wnt3a, growth factors such as TGF (transforming growth factor) (3, FGF (fibroblast growth factor), EGF (epidermal growth factor, Hedgehog, etc.; Affinity binging pairs are known in the art, and include, but are not limited to Fc-protein A/G, albumin binding domain (ABD)-serum albumin (SA), barnase-barster, His-Ni-NTA) with MMM platform.

In one embodiment of this disclosure, a micro-substrate of an inert protein such as bovine serum albumin (BSA) is fabricated on a solid substrate such as glass by two-photon induced photochemical crosslinking, followed preferably in the form of micromatrix, by micropatterning a first affinity pair member-bioactive factor/protein (for example) biotin-binding protein, the second affinity pair member (for example, neutravidin (NA)), in a pre-defined ROI on the surface of the inert protein micro-substrate. I, the first affinity pair member-bioactive factor/protein (e.g., biotin-conjugated soluble factors (e.g., BMP2 or Wnt3a)) are applied to micropatterns including the second affinity pair member (for example NA, when the bioactive factor is conjugated to biotin to form a soluble factor micropattern via affinity pair (biotin-NA) non-covalent binding. In addition to the spatial controllability, the local density of soluble factor micropatterns can be precisely controlled by varying either the laser parameters or reagent parameters in the process of micropatterning one affinity pair member (e.g. NA), which could not be achieved using conventional methods.

First Affinity Pair Member Micropatterning

Micropatterns of the first affinity pair member such as neutravidin are generated by two-photon induced photochemical crosslinking, in which neutravidin is covalently bonded together. As such, the reliability of binding biotin molecules to the micropatterned neutravidin is virtually guaranteed, as evidenced by the observation that the biotin-binding capacity of the neutravidin micropatterns do not change over 4 weeks' time. Again, the method to micropatterning neutravidin disclosed herein is able to quantitatively control the local density of neutravidin thereby quantitatively controlling the biotinylated soluble factors to form a gradient, which cannot be realized by conventional methods.

### Soluble Factor Conjugated to Second Affinity Pair Member Micropatterning

The soluble factor is preferably conjugated to the second affinity pair member prior to micropatterning, using methods known in the art, and exemplified in the Examples for biotinylation of BMP-2. The soluble factor conjugated to second Affinity pair member, for example, biotin-binding protein, neutravidin (NA), is immobilized in a sophisticated arbitrary region of interest on the surface of a prefabricated BSA micro-substrate generated on the glass by two-photon induced photochemical crosslinking, in a spatial- and quantity-controlled manner. Then the biotin-conjugated soluble factors (e.g., BMP2 or Wnt3a) are applied to the NA micropatterns to form a soluble factor micropattern via biotin-NA non-covalent binding. in one embodiment 2.5D protein-based microstructures are made, wherein the bulk properties, topological features as well as chemical properties can be feasibly tuned by laser parameters, reagent parameters as well as free-written ROIs, to recapitulate a biomimetic cell niche in vitro. Unlike other methods which require sophisticated modifications on both bulk material (e.g., agarose gel) or one party of binding pairs (e.g., barnase and streptavidin) of conventional methods, the disclosed methods allow the quick and easy micropatterning of pristine biotin-binding protein (e.g., neutravidin) thus subsequently micropattern biotinylated soluble factor.

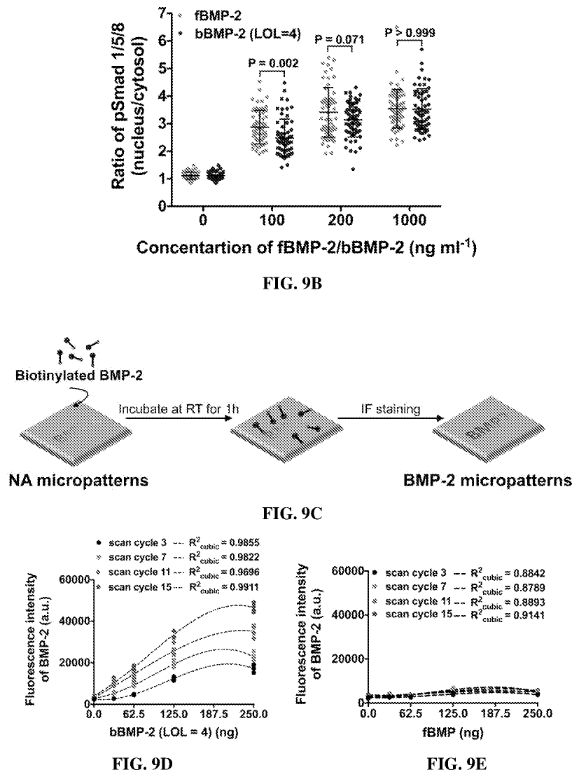

The method includes the steps of (i) fabricating a micro matrix comprising a neutral protein on a solid substrate; Such substrates are commercially available, for example, Silicone micro-inserts (ibidi, #80409, Munich, Bavaria, Germany) (well dimensions in mm (2.0×1.5) sticking onto a 35 mm glass-bottomed dish (MatTek, P35G-1.5-14-C, Ashland, MA, USA) by contacting the solid substrate with a solution containing the inert protein and a photosensitizer such as rose Bengal. The inert protein BSA protein substrate in the form of flat micro-matrix with dimension such as 101×101×1 μm (length×width×height) is first fabricated by z-stack scanning of the 800 nm two photon laser through the bulk protein substrate solution from 1 μm below to 1 μm above the zero position as disclosed in the Examples. The fabricated micro matrix is then contacted with a solution containing a first member of an affinity pair such as NA and a photosensitizer, for crosslinking. Multiphoton laser scanning is used to fabricate a micropattern first member of an affinity pair such as NA. The solid substrate functionalized with a matrix of inert protein on which the first binding partner of an inert pair has been micropattern is then incubated with a soluble factor conjugated with the second member of the affinity pair (FIG. 9C).

The disclosed methods avoid tedious steps such as steps to activate the glass or to synthesize PVA monolayer film, and more importantly, there are no toxic solvents involved in the whole fabrication process, while several toxic solvents including glutaraldehyde are used in the glass activation in conventional methods. The disclosed soluble bioactive factor functionalization method can be extended to multiple applications, including but not limited to, gene microarray fabrication, engineering soluble cell niche with counter-gradients of morphogens/antagonists and solid-phased drug/biomolecule biochip fabrication.

Compared with conventional methods, the platform described herein is advantageous on: (1) free-writing arbitrary structures with sub-micron feature size; (2) bulk properties and functional properties of the structure can be readily decoupled via crosslinking a thin layer of neutravidin on the surface of BSA bulk structure, thereby allowing to study the impact of a specific niche factor on cell behaviors; (3) flexibly controlling the local density of crosslinked neutravidin so as to control the amount of biotinylated soluble factors attached by varying either the laser parameters or reagent parameters in the process of micropatterning NA; (4) more sustained and higher level of downstream signaling events triggered by NA-bound soluble factors (e.g., BMP-2 and Wnt3a) are demonstrated, justifying the feasibility of this platform in reconstituting cell niche in vitro.

### III. Biochip Screening Platform and Multiplexed Biochips Resulting Therefrom

In one embodiment, the subject matter described herein involves the multi-stage screening phase, including:
a preparation phase comprising isolating cells and determining screening parameters of the isolated cells, including but not limited to cell validity, experiment duration, and screening readouts;
a first screening tool, preferably an MMM fabricated biochip, comprising various cell niches for cell culture, wherein each set of niches contains only one varying niche factor at different levels and to identify at least one effective niche factor; and
a second screening tool, preferably an MMM fabricated biochip comprising combinatory cell niche designs is generated and used to test their effect on test cell-specific predetermined characteristics.

Another embodiment provides an imaging data handling software comprising a Matlab-based algorithm for effective management and processing of massive images with repeatability and reproducibility.

In another embodiment, the subject matter described herein involves the applications including but not limited to, screening applications with the biochips are used to investigate of various cellular activities including but not limited to phenotype maintenance, proliferation, apoptosis and differentiation under specific cell niches; drug screening in different physiological and pathological niches; functional assays for a particular gene, which is knocked out or down in cells as compared with the wild type cells, under specific cell niches. Information generated from the cell niche screening can be used in the design for cell-specific culture substrates and cell-specific scaffolds; and/or an example of cell niche screening by phenotype maintenance of bovine nucleus pulposus cells.

A method of making biochips incorporating optimal niche factors that mimic the in vivo environment of a cell or group of cells (test cell or cells) is disclosed. The test cell can be any cell of interest, and include, but are not limited to chondrocytes, hepatocytes, nucleus pulposus cells (NPCs), tumor cells, adult stem cells, embryonic stem cells, mesenchymal stem cells induced pluripotent stem cells, adipocytes, etc.

The method is a systematic and high-throughput cell niche screening platform including two stages. Using a two stage screening process, an optimal combination of cell niche factors in maintaining the phenotype of a test cell or cells, in culture, is obtained. This optimal combination of cell niche factors is incorporated into a biochip preferably using multiphoton micropatterning and microfabrication (MMM), to provide multiplex protein biochips. Multiple sets of cell niche factors are incorporated into a biochip design including microwell, with each microwell incorporating only test cell niche factor in the first stage. The method also includes biochip fabrication, wherein a tailor-made chip design is provided, which includes a combination of cell niches factors selected from the first line of screening, incorporated into microwells of a biochip.

In a preferred embodiment the cell niche factors are incorporated into a biochip using the multiphoton micropatterning and microfabrication platform to arbitrarily control the various niche properties, including but not limited to mechanical, topological and biochemical properties, by iterative fabrication approach. The materials for cell niches can include any materials that can be crosslinked by MMM including but are not limited to bovine serum albumin and extracellular matrix proteins.

Microstructure Microfabrication Using MMM Technology

Multiphoton micropatterning and microfabrication platform (MMM), which is a 3D-microprinting technology able to arbitrarily construct microstructure made of materials including but are not limited to protein and enable the control of mechanical properties (such as modulus), biochemical properties (by functionalization with extracellular matrix proteins) and the arbitrary topological features (by microfabricating the pre-determined ROI). The platform possesses both microfabrication and micropatterning capability and is able to independently control the niche properties easily and engineer multiple cell niche factors within the same platform. Apart from that, a number of advantages of this platform are highlighted in the field of cell niche engineering, including the biocompatibility, multiplexity and submicron resolution. The capability to decouple multiple niche factors during fabrication especially favors its applications in both studying individual cell niche factors and engineering complex cell niches. In the multiphoton microfabrication and micropatterning platform, protein molecules can be readily crosslinked and solidified by the high-power infrared laser, with the use of photo sensitizers. Multiplex cell niche factors can therefore be incorporated independently, including but not limited to topological, mechanical and matrix niche factors.

As shown in FIG. 1B, for multiphoton microfabrication (MMM), a protein solution such as bovine serum albumin (BSA), is mixed with photosensitizer (for example Rose Bengal), and an aliquot placed on a suitable substratum such as glass in a biochip is placed on a xyz-controllable stage of a fabrication machine or a confocal microscope. Photochemical crosslinking of the protein is induced by the femto-second infrared laser at 800 nm for example, via the direct and indirect mechanism with the photosensitizer. Femto-second laser at a wavelength such as 800 nanometer is controlled to excite specific spots in the solution. Making use of the arbitrary spatial and intensity control of the laser spots together with the stage, microstructures with arbitrary topological architecture and mechanical strength are generated on the glass substratum of the biochip. Microstructure can be fabricated by layer-by-layer scanning by the laser from the bottom of the glass substrate, after locating the solution-glass interface by reflective mode autofocusing. The focusing of IR laser in the 3D space and the multiphoton excitation enable the construction of arbitrary architectures, while the xyz-movable stage allows the fabrication to be conducted and controlled in a large area as a biochip form.

The mechanical strength in terms of modulus can be simply controlled by the output power of the laser in a linear relationship, while the laser movement can be controlled as region of interest (ROI) in confocal microscope setting to enable the arbitrary 3D microstructure generation or the stiffness control of a micropillar array.

The microstructure design can be prepared with the microfabrication platform interface or the computer-aided design (CAD) software with custom-made codes. The stiffness of the cylindrical micropillar array can be calculated by the formula below (Equation 1). For the part of micropatterning, the diagram on the right depicts the later steps in functionalizing the microstructure with biomolecules. The fabrication solution is changed to the mixture of the targeted biomolecules and the photosensitizer after several times of washing with phosphate-buffered saline.

Biomolecules that can be immobilized by the same approach for functionalization include but are not limited to extracellular matrix proteins such as fibronectin and laminin, the linker proteins such as protein A/G and biotin, and soluble molecules. The functionalization procedures are done by the same MMM platform separately, from the microstructure construction. However, the regions for photochemical crosslinking are confined to the microstructure surface by appropriate ROIs, to prevent the alteration of mechanical properties and thus to provide independent magnitude and spatial control. Furthermore, by varying the laser properties and the protein mixture, the 3D spatial and dosage control of the biomolecules on the microstructure surface can be achieved, as shown in the diagram.

$$stiffness ( s ) = \frac{\begin{matrix}3 \times ( elastic modulus ( E ) ) \times \\ ( second moment of area ( I ) )\end{matrix}}{{( pillar height ( L ) )}^{3}} = \frac{3 EI}{{L}^{3}} , \qquad equation ( 1 )$$

$$where second moment of area ( I ) = \frac{π {a}^{4}}{64}$$

The disclosed multiplex cell niche engineering platform enables systematic screening of cell niche factors for enhancement of a particular type of cellular activities or cellular fate processes, exemplified by phenotype maintenance in the examples and in a particular cell type, exemplified by bNPCs in the examples.

The three main phases in the entire pipeline include preparation phase, multi-stage screening phase and further investigation. The last phase is for further study such as the cell response to drug in a biomimetic phenotype-maintaining niches, and thus is not within the niche screening. Different products and services can be offered to the customers at different phases. Basically, the products include the biochips (in standard, tailor-made and pure specific niche chips), the screening protocol, and an imaging data management software. The available services include the consultation to the preparation phase, the design and preparation of tailor-made chip and pure niche chip, and the data processing and reporting services.

Preparation Phase

A preparation phase typically determines and rationalizes the screening parameters in terms of cell validity, experiment duration and screening readouts used in the screening phase (FIG. 1C). The general approach is to examine the native tissues or cellular activities in native tissues or in conventional cell culture as the reference, typically by conventional assays like immunostaining and western blots. In the case of phenotype maintenance, it is recommended to include native tissues, native cells and dedifferentiated cells in the preparation phase. The native tissues and the cells isolated from these tissues are first evaluated to ensure their quality by means such as their marker expression levels. Later, examining the response of the interested cell types in terms of the targeted cell behaviors is to determine the necessary screening parameters including but not limited to the cell entities, the experiment duration and the measurable readouts to be used in the coming screening phases. The isolated cells are cultured as 2D monolayer and used to verify whether their de-differentiation can be characterized by the expressions of selected phenotype markers such that their expressions can reflect the dedifferentiation state and act as readouts within the experiment duration. Furthermore, multiple phenotype markers are recommended as readouts so as to increase the reliability of the screening results as the probability to have multiple false-positive results simultaneously should be extremely small.

FIG. 1A is a schematic diagram illustrating the overall experimental design of the MMM-based multiplex cell niche screening, using a two-stage screening phase covering microfabrication of protein culture substrates with multiple cell niche factors.

First Screening Stage

The first line of screening can be conducted with the standard biochip format in which individual niche factors at various levels are presented in each niche set. With the niche factor decoupling property of the MMM platform, each cell niche has only one of the niche factors varies at different levels so as to work out the best dosage of the specific niche factor in stimulating or inducing or maintaining the specific cellular activities, phenotype maintenance as an example, at optimal level.

Cell/cells of interests are cultured on these protein substrates with specific cell niche factors at various levels for a specific duration as determined in the preparation phase. The performance of phenotype maintenance can be evaluated by the expression of selected markers with immunofluorescence staining and imaging modalities such as confocal microscopy. The imaging data collected are then analyzed for the fluorescence intensity in the single-cell level.

Individual cell niche factors that are effective in stimulating or inducing or triggering or maintaining the specific cellular activities and their optima levels are shortlisted in this stage. The shortlisted factors are then combined into multiple complex combinatory cell niche designs, which are to be validated or further fine-tuned in the second line screening stage.

The first phase includes microfabricating protein microstructures incorporated with multiple types of niche factors (for example, topological, mechanical and ECM (extracellular matrix) factors at different levels) known for the test cell/cells, culture the test cell/cells on the engineered cell niches and evaluating their phenotype maintenance using characteristics such as cell morphology and phenotype marker expression known for the test cell/cells, as the first line screening for individual cell niche factors. Among multiple types and multiple levels of cell niche factors, the first stage screening exemplified herein using bNPC shortlisted a number of individual cell niche factors, including low stiffness among the mechanical niche factors, fibronectin and vitronectin among the matrix niche factors, micropillar array and fiber-beads micropatterns among the topological niche factors, with significantly upregulated in bNPC phenotype markers.

In the first stage of screening, a test cell/cells are cultured on culture substrates such as microstructures fabricated using MMM that include individual niche factors for a period of time effective for expression of cell type specific characteristic factors such as morphology and cell-specific receptors, followed by phenotype evaluation to determine the effect of the niche factor on cell type specific characteristic factors (FIG. 1A). In the second line of screening, niche factors shortlisted from the first line of screening are combined on culture substrates such as microstructures fabricated using MMM, that include combinations of niche factors identified in first screening stage. The test cells for a period of time effective for expression of cell type specific characteristic factors such as morphology and cell-specific receptors, followed by phenotype evaluation to determine the list of niche factors which support optimal of cell type specific characteristic factors

Exemplary Cell Niche factors that can be screened are shown in Table 1

Second Screening Stage

In the second stage, using the phenotype-maintaining cell niche factors shortlisted from the first stage of screening (for example 1 mechanical, 2 topological and 2 ECM factors), protein microstructures integrating the shortlisted cell niche factors in combination are reconstituted and used to verify the optimal phenotype maintenance of the test cell/cells. Using the same MMM platform, these shortlisted cell niche factors are combined during the fabrication process to create complex combinatory protein cell niches, for example, vitronectin and laminin functionalized micropillar arrays, or vitronectin and laminin functionalized fiber-beads microstructures as identified in the first screening stage for bNPCs.

After collecting the list of shortlisted niche factors, these complex combinatory cell niche designs may present as the tailor-made biochip format for the second-line screening. The performance of these complex niche designs are further evaluated in this second-line screening by the same approach. Further optimizations of these complex niche factor level can be done by iteratively fine-tuning the constituent niche factor composition and levels until the optimal cellular activities are achieved.

Matlab-Based Programme

Also provided is a Matlab-based programme that can be integrated with the fabrication facility to automate the screening and the analysis with high throughput and repeatability. This requires testing, optimization and validation. This tool written in Matlab is a framework with a user interface to organize and semi-automate the data management, image analysis sequences and data presentation, when managing huge amount of imaging data. The framework currently includes the essential functions but are free and ready to upgrade or incorporate new codes. This tool may be a useful accessory tool for the cell niche screening platform, which includes huge amount of screening data. Besides, currently the analysis of results is based on imaging of stained markers in cells. This means that only the protein markers are considered and the number of markers to be evaluated is very limited, by the number of available antibodies available. A better and high throughput understanding of the status of cells, single cell gene profiling can give even better results.

The Examples show results from the study using the MMM platform to screen for the phenotype-maintaining niche for bNPCs. Data from the first-line screening, shortlisted the niche factors such as stiffness, ECM proteins (including laminin and vitronectin) and topological features for the next stage, by the enhancement of phenotype marker expressions including COL2, KRT8 and SNAP25. The right panel then demonstrated the results from the second-line screening stage that the complex niche designs generated from the shortlisted niche factors significantly outperform the conventional culture condition in maintaining the cell phenotype. These results demonstrated the multi-stage screening as an efficient and effective approach in handling the hierarchal composition of cell niches.

The screening platform and procedures have been elucidated with bNPC phenotype maintenance as an example. However, this biochip platform does not solely applicable to phenotype maintenance and screening applications. The current multiplex cell niche screening platform enables the design of an optimal cell niche for in-vitro phenotype maintenance of cells, which are otherwise poorly maintained by the conventional 2D monolayer culture. Apart from bNPCs, many cell types including but are not limited to hepatocytes, chondrocytes, prostate tumor cells and mesenchymal stem cells which can barely survive or difficult to maintain in monolayer cultures, would also be able to benefit from this cell niche screening platform before defining suitable culture substrates or conditions. Besides, the current platform can be used to screen for the optimal cell niche designs for cellular fate processes other than phenotype maintenance, to list a few examples, proliferation, migration and differentiation, stem cell differentiation, neuron processes elongation and extension, survival and proliferation of slow-dividing cells, and asymmetric cell polarity establishment. Furthermore, cells residing in a cell niche favorable to the phenotype maintenance of their in-vivo conditions is more likely to generate in-vivo-like and physiologically relevant responses. Therefore, apart from generation of phenotype-maintaining in vitro culture substrate as demonstrated in the current work, screening phenotype-maintaining cell niches also contributes to develop physiologically-relevant in vitro culture platform for predictable drug screening and rationalizing biomimetic scaffold design for functional tissue engineering.

Utilizing the multiphoton microfabrication and micropatterning (MMM) platform, this study reports an effective and high-throughput biochip system for screening multiplex protein-based cell niches for induction of different cell behaviors. The biochip designs and method of preparation are disclosed, together with the proposed product and service pipeline. The systematic niche screening approaches containing the preparation phase, multi-stage screening phase and the downstream investigations are also elaborated, with a list of possible biological applications with this technology. All in all, this multiplex platform enables high throughput screening of cell niche factors for enhancement of a particular type of cellular activities or cellular fate processes, exemplified by phenotype maintenance in this work, and in a particular cell type, exemplified by bNPCs in this work. This work contributes to future applications such as reconstituting biomimetic cell niche to tailor-made cell culture substrates for physiologically relevant cell cultures and drug screening, as well as optimizing biomimetic scaffold design for stem cell differentiation and functional tissue engineering.

### Examples

### 1. Multiphoton Microfabrication and Micropatterning (MMM)-Based Screening of Multiplex Cell Niche Factors for Phenotype Maintenance—Bovine Nucleus Pulposus Cell as an Example

Multiphoton Microfabrication

Instruments, materials and microfabrication A mixture of bovine serum albumin (BSA) and photosensitizer Rose Bengal (RB) was freshly prepared in 1× phosphate buffered saline (PBS) at a final concentration of 330 mg/mL and 0.2% (w/v), respectively. An aliquot of 10 μL of the BSA/RB mixture was added to a 35 mm glassbottom dishes (MatTek #P35G-1.5-14-C) before loading onto the xyz controllable stage. A near infrared (NIR) laser at 800 nm wavelength and a 40× oil immersion objective lens of 1.3 numerical aperture (NA) were used for the microfabrication and micropatterning. The output laser power for microfabrication was measured by a power meter (Coherent) before each experiment. The instrument was controlled by the ZEN2010 software (Carl Zeiss). Major microfabrication parameters including laser power, scan cycle and regions of interest (ROIs) for laser scanning were controlled by the ZEN2010 software interface. Reflective mode autofocus function was used to locate the glass-solution interface and define the initial z-plane for microfabrication. Excess BSA/RB solution was discarded after microfabrication and the microfabricated protein microstructures and micropatterns were thoroughly rinsed thrice with excess PBS.

Mechanical Niche Factor—Microstructures with Different Mechanical Properties

Microstructures with different modulus and stiffness were presented as BSA flat matrix and micropillar array respectively. Microfabrication of BSA protein microstructures (flat micro-matrices and micropillar arrays) with controllable elastic modulus and stiffness through a series of fabrication parameters was reported previously. To fabricate flat protein micro-matrices with different mechanical properties, BSA/RB mixture was loaded onto the glass-bottom dish for microfabrication as described in Materials and Methods. The elastic modulus of the resultant flat protein micro-matrices ranging from 10 kPa to 50 kPa was tuned by adjusting the laser power between 110 mW and 190 mW at 800 nm. Flat protein micro-matrix blocks of 5 μm height were micro-fabricated by z-stack scanning at 0.5 μm z-axis scanning interval, at selected power. The time required is approximately 1.5 min for a 300×300 μm area. To micro-fabricate protein micro-structures with controllable stiffness, a design of micro-pillar array rather than flat micro-matrix was used. Specifically, the micro-fabrication process used was similar to that of the flat protein micro-matrices described above, except that a region of interest (ROI) file (.ovl format) was imported to the ZEN2010 software prior to micro-fabrication. Specifically, microfabrication was conducted by repeated scanning of a ROI of 2 μm-diameter circle with 2 μm edge-to-edge gap, giving rise to the creation of a protein micropillar array. By adjusting the height (4-16 μm) of the micropillars, micropillar arrays of different stiffness were obtained. The stiffness was calculated by the following equation. The time required is approximately 2.5-15 min for a 300×300 μm area, depending on the height of the pillars.

$$stiffness ( s ) = \frac{\begin{matrix}3 \times ( elastic modulus ( E ) ) \times \\ ( second moment of are ( I ) )\end{matrix}}{{( pillar height ( L ) )}^{3}} = \frac{3 EI}{{L}^{3}} \qquad ( 1 )$$

$$where second moment of area ( I ) = \frac{π {a}^{4}}{64}$$

Matrix Niche Factor—Micro-Matrices Functionalized with Different Extracellular Matrices (ECMs)

The surface functionalization of microstructures was done by photochemical crosslinking with the mixture of ECM protein and photosensitizer. Flat BSA protein micro-matrices were microfabricated as described in Materials and Methods. In Brief, BSA solution (300 mg/mL) and 0.1% RB (w/v) were photochemically crosslinked using a square ROI (of 100 μm×100 μm) at a laser power of 190 mW and the thickness of the flat micro-matrix fabricated was 5 μm. After three washes with PBS, the flat BSA micro-matrices were functionalized with different ECM proteins as demonstrated previously. In brief, an aliquot of 5-10 μL ECM/RB fabrication solution, which was freshly prepared from specific ECM, 1×PBS and RB (0.1% w/v), was added to the sample. ECM protein solution was prepared from ECM in 1×PBS. The final concentrations of the ECM protein solution were 1 mg/mL for fibrinogen (Fg; Sigma #F3879), 0.9 mg/mL for fibronectin (Fn; Sigma #F-2006), 0.9 mg/mL laminin (Lm; Corning #354232) and 0.45 mg/mL for vitronectin (Vtn; Gibco™ #PHE0011), respectively. The same multiphoton microfabrication process was used to functionalize or surface coat the protein micro-matrices with ECM. The time required is approximately 1.5 min for a 300×300 μm area.

Topological Niche Factor—Microstructures with Different Topological Features

The microfabrication of various 3D topological features involved the use of CAD software, a custom program to generate sets of ROI files, and the layer-by-layer fabrication. To fabricate protein cell niche with different 3D topological features, a custom program was written by Matlab to convert 3D objects either drawn by CAD software (such as SolidWorks and AutoCAD) or generated directly by Matlab to ROI files (.ovl format) for microfabrication. A fabrication solution consisting of BSA (330 mg/mL) and RB (0.2% w/v) was used for fabrication of protein microstructures with different topological features. A wide range of topological features including simple and hierarchical grating structures, convex, concave structure, micropillar arrays and random fiber-beads structure, were designed and made into ROI files. Protein cell niche with different topological features were micro-fabricated with multiple ROIs for different z-levels. The time required was approximately 2-7.5 min for a 300×300 μm area, depending on the variability between layers. The fabricated protein cell niche with different topological features were ready for subsequent scanning electron microscopy (SEM) evaluation.

To visualize the ultra-structure of the micro-fabricated protein cell niche with various topological features, protein microstructures with different topological features were assessed by SEM. In brief, the microstructures were fixed by 2.5% glutaraldehyde (GTA) at room temperature for 15 min in darkness, followed by thorough washing with PBS for three times. After serial dehydration with different ethanol concentrations, the samples were processed with critical point drying followed by 100-s gold sputtering. SEM images at different magnifications were taken with the scanning electron microscope (Hitachi S3400 N & S4800; HKU EM unit).

Combinatory Protein Cell Niche Integrating Topological and ECM Factors

Complex niches with designed mechanical, ECM and topological properties were investigated after the first line of screening. In the first line of screening, the type and level of individual mechanical, ECM and topological cell niche factors in optimally maintaining bNPC phenotype were identified. In the second line of screening, selected mechanical, ECM and topological cell niche factors were combined for fabrication of protein microstructures and micro-patterns with combinatory cell niche factors before evaluating their effects in phenotype maintenance of bNPCs as described in subsequent section (Materials and Methods). Based on the results of the first-line screening, protein microstructures with medium modulus and low-to-medium stiffness were used in fabricating the protein culture substrates of combinatory cell niches in the second line screening. Two individual topological cell niche factors, namely micropillar array and fiber-beads were shortlisted from the first line screening and were incorporated into the protein culture substrates with combinatory cell niche factors fabricated in the second line screening. The protein culture substrates with the selected topological features were microfabricated first, followed by three washes of PBS before refilling with freshly prepared ECM/RB solution containing two shortlisted ECM (Laminin and Vitronectin) for functionalization or surface coating. Specifically, ECM/RB solution containing Laminin (Lm) (0.45 mg/mL) and Vitronectin (Vtn) (0.225 mg/mL) in 1×PBS and RB (0.1% w/v) was supplemented to the culture substrates for multiphoton-based surface coating. The same ROI used for the fabrication of the topological features was recalled and a thin layer of ECM microstructures with the same topological feature was fabricated by z-stack scanning of the multiphoton IR laser at 2 μm z-interval and at 48 mW for 7 scan cycles. Functionalization took approximately 3.5 min for a 300×300 μm area. Flat BSA protein micro-matrix coated with BSA/RB and 2D culture dish were used as the control culture substrates in the second line screening. The maintenance of bNPC phenotype in the combinatory cell niche groups and the control groups were evaluated through immunofluorescence staining of the selected phenotype markers, followed by quantitative intensity measurement.

Harvest of bNP Tissues, Culture of bNPCs and Phenotype Characterization

Harvesting bNP Tissues

The bovine nucleus pulposus (bNPs) tissues were harvested from adult bovine caudal discs of the tail as previously described [58]. In brief, fresh adult bovine (1-2 years old) tails were purchased from local slaughters. The gel-like NPs were isolated by sterile surgical scalpels and scissors. Some bNP tissues were paraffin-embedded and then characterized by histological (H&E), histochemical (Safranin O staining), and immunohistochemical (collagen type I and type II) staining. Other bNPs tissues were cryo-embedded for immunofluorescence (IF) staining for the selected panel markers for bNPCs (COL2, KRT8 & SNAP25) and F-actin.

Isolation of bNPCs

The freshly isolated NPs were cut into small pieces and digested enzymatically by 0.25% pronase (Sigma Aldrich) for 1 h followed by digestion in 4.8 mg/mL collagenase II (Sigma Aldrich) for 8 h, at 37° C. in darkness. The digestion mixtures containing bNPCs were then collected through cell strainers of 70 μm pore size and washed with medium for thrice before culture expansion as monolayers.

Monolayer Culture Expansion of bNPCs and Phenotype Characterization

Primary bNPCs were used in all experiments. Low glucose Dulbecco's modified Eagle's medium (DMEM) with 10% fetal bovine serum (FBS; Gibco™) and 1% Antibiotic-Antimycotic (Gibco™ #15240) was supplemented to 100 mm TC-treated culture dishes (Corning) for bNPC culture. The culture medium was replenished every 3 days. The bNPCs in monolayer cultures were fixed in 4% paraformaldehyde (PFA) in dark for 10 min and characterized in terms of growth kinetics, IF staining and Western blot for the selected bNPC panel marker proteins (COL2, KRT8 & SNAP25) to monitor the changes in their phenotype, specifically, reduced expression of bNPC phenotype markers, upon dedifferentiation at different time points during monolayer cultures. This defines the duration of monolayer culture and the composition of the panel marker proteins to be used for the subsequent cell niche screening. Details of the staining involved are provided (Materials and Methods Section).

bNPC Culture on 3D Protein Cell Niches and Phenotype Characterization

bNPCs (5×10⁴ cells/mL) were seeded onto protein culture substrates incorporated with various cell niche factors microfabricated and micropatterned on 35 mm glass-bottom dishes. The culture conditions were the same as those for monolayer cultures. After 7 days of cultures, samples were fixed in 4% PFA for 10 min in dark and the phenotype of the bNPCs was characterized by IF staining against the same bNPC panel markers as that in the monolayer cultures (Materials and Methods Section).

Characterization of bNP Tissues by Histology and Immunohistochemistry

Bovine nucleus pulposus tissues were embedded in paraffin wax and sectioned at 10 μm thickness. Routine haematoxylin and eosin staining was used to reveal the cell morphology and distribution in the tissue. Safranin-O staining counter-stained with haematoxylin QS was used to evaluate the IVD status in terms of amount of extracellular sulfated glycosaminoglycan. For immunohistochemical staining, the paraffin sections were undergone antigen retrieval by 95° C. water bath in sodium citrate buffer for 10 min, after dewax and rehydration. The samples were then treated with 3% hydrogen peroxide in methanol for 30 min after washing, to block the endogenous peroxidase activity. It was followed by PBS wash and 1.5% goat serum blocking for 60 min. They were then incubated with primary antibody solution (anti-COL1 and anti-COL2, Abcam) in blocking buffer at dilution of 1:200. After incubating at 4° C. overnight, the sample were then incubated with biotinylated anti-rabbit secondary antibody at dilution of 1:400 for 30 min at room temperature. The positive expression of targeted protein was visualized by Vectastain ABC kit and Vector BAB substrate. The sample was counterstained by haematoxylin QS before being mounted with Depex.

Characterization of bNP Tissues and bNP Cells by Immunofluorescence (IF) Staining

bNP tissues were embedded in cryomatrix and sectioned at 10 μm thickness. bNPCs were cultured onto sterile glass coverslips and were fixed with 4% paraformaldehyde after 4-day and 7-day culture. The samples were permeabilized with 0.5% Tween20 for 10 min and blocked by 3% BSA for 30 min in room temperature. Primary antibodies and their dilution includes anti-collagen type II (anti-COL2; Abcam #ab34712; 1:100), anti-keratin 8 (anti-KRT8; Abcam #28050; 1:100), anti-SNAP25 (Merck Millipore #ab1762; 1:200), anti-integrin α6 (ab105669; 1:200) and anti-poliovirus receptor (anti-PVR/anti-CD155; Abcam #103630; 1:200). After incubation with primary antibody solution at 4° C. overnight, secondary antibody (Alexa Fluor 647-tagged anti-rabbit, 543-tagged anti-mouse, 488-tagged anti-rat) at dilution 1:400 was used according to the primary antibody host, together with 488-tagged Phalloidin for F-actin visualization. The samples were mounted with DAPI-containing mounting medium after 1.5 h secondary antibody incubation.

Characterization of bNP Cells' Marker Protein Expression by Western Blot

To evaluate the protein marker expression during monolayer culture and decide the appropriate experiment length, Western blotting of the bNPC molecular marker COL2, KRT8 and SNAP25 was performed at different time points of monolayer primary culture (after isolation, day1, 4, 7, 10, 14). For the Western blot part, bNPCs cultured in cell culture dish (Corning) were lysed at day 0, 4, 7, 11 and 14 and were stored at □80° C. before performing Western blot. Cells on culture dish were rinsed with PBS and trypsinized with 0.05% Trypsin/EDTA (Sigma Aldrich) for 5 min at 37° C. After counting with haemocytometer, the cell pellet was lyzed by ice-cold RIPA lysis buffer with protease and phosphatase inhibitors (Abcam #ab156034; Cytoskeleton #PIC02; Merck Millipore #524625). The total protein concentration was estimated by bicinchoninic acid (BCA) assay (ThermoFisher; Pierce BCA protein assay kit). The protein mixture of different timepoints were separated by electrophoresis on 10% polyacrylamide gels. Each lane was loaded with 20 μg total protein in SDS buffer. The separated proteins were transferred to PVDF transfer membrane (GE Healthcare #RPN303F; 0.45 μm). The membrane was then blocked with 3% BSA in TBST for 1 h and immune-stained by antibodies (anti-COL2, Abcam #ab34712, 1:5000; anti-KRT8, Abcam #28050, 1:1000, anti-SNAP25, Merck Millipore #ab1762, 1:2000, anti-GAPDH, Abcam, 1:5000) in the blocking buffer at 4° C. overnight. After washing with TBST, the samples were incubated with 1:5000 HRP-conjugated anti-rabbit/anti-mouse IgG secondary antibodies (ProMega #W401B; #W402B) for 1 h at room temperature. The chemiluminescence signals by reactions between HRP and substrate (ThermoFisher #34077) were captured by Azure Biosystems C300. The protein levels were quantified as intensity sum in arbitrary units. Three independent experiments were performed.

bNP Cell Growth Kinetics During In-Vitro Monolayer Culture

NP cells from three bovines were seeded onto different 6-well plates (Corning) at density of 1000 cells/well. The cells were trypsinized at day 2, 5, 8 & 11. The cells already reached 100% confluence (˜3.2e6 per well) before day 11. Thus that data point was not shown in the results. The data were displayed as log scale after normalization to the initial cell seeding density (1000 cells). The average doubling time was also calculated.

Evaluation of Screening Samples

Florescence (IF) staining, image acquisition and intensity quantification, together with statistical analysis with IBM SPSS Statistics. Quantitative data are presented as mean±95% confidence interval. One-way ANOVA with post-hoc test was adopted with statistical significance of 0.05.

Immuno Fluorescence (IF) Staining

For a high-throughput microscale multiplex screening using miniaturized protein micro-niches, IF staining rather than conventional protein assays such as western blotting was selected to evaluate the expression of phenotypic markers, because the latter requires a huge number of cells. In particular, cell-seeded protein cell niche samples on glass-bottom dishes was rinsed thrice in PBS (5 min each) after discarding the culture medium. The samples were then fixed with 4% paraformaldehyde (PFA) in dark for 10 min and were permeabilized with 0.5% Tween-20 for another 10 min. Blocking of non-specific binding was done by incubation with 3% BSA for 30 min. The sample was immediately incubated with primary antibodies at specific dilutions in 3% BSA at 4° C. overnight. Primary antibodies including anti-collagen type II (anti-COL2; Abcam #ab34712; 1:100 dilution); anti-keratin 8 (anti-KRT8; Abcam #28050; 1:100 dilution) and anti-SNAP25 (Merck Millipore #ab1762; 1:200 dilution) were used. After primary antibody incubation, Alexa Fluor 647-tagged goat anti-rabbit and goat anti-mouse secondary antibody (Invitrogen #21245; #A21236) were used at 1:400 dilution in 3% BSA for rabbit and mouse primary antibodies, respectively. Alexa Fluor 488-tagged Phalloidin (Invitrogen #A12379) was also added to the solution at 1:40 dilution to visualize the actin filaments and cell morphology. The samples were finally mounted with Fluoro-gel II mounting medium containing DAPI. bNPCs cultured on flat BSA micro-matrix were used as the control groups in subsequent experiments.

Image Acquisition and Data Analysis

Images were acquired with the same laser scanning confocal microscope (Carl Zeiss LSM710, Oberkochen), with a 40× oil immersion objective lens of 1.3 numerical aperture (EC Plan-Neofluar, Carl Zeiss). The imaging parameters such as resolution (below half of the deflection limit) were set to increase the signal to noise ratio for intensity quantification [59]. A z-stack image of 12.5 μm thickness was captured for full thickness imaging of whole cells. The images were then analyzed by Matlab. Shading correction was done to compensate the uneven illumination effect. Subsequent segmentation of micropatterns and cells were performed to obtain the mask of the cells by the standard auto-thresholding method to the 543 nm Rose Bengal and the 488 nm F-actin staining. The segmentation procedures were validated by manual segmentation and Imaris (Bitplane, Oxford Instruments) segmentation function in advance. The intensity values of the phenotype marker IF staining measured were normalized to that of the control groups (bNPCs cultured on BSA micro-matrices). Additional measurements of the cell morphological information such as the 2D roundness and the aspect ratio of the maximum intensity projection were calculated by equations (2) and (3) respectively.

$$roundness = \frac{4 π \times area}{{perimeter}^{2}} \qquad ( 2 )$$

$$aspect ratio = \frac{long axis length}{short axis length} \qquad ( 3 )$$

Statistical Analysis

Quantitative data are presented as mean±95% confidence interval. All groups included the measurement of at least 30 independent cells from 3 independent experiments from 4 independent bovine samples. Normality of the sampling distribution was assumed by applying the Central Limit Theorem owing to the fact that sample size >30 and therefore parametric tests were used. One-way ANOVA with Bonferroni's post-hoc test was performed unless otherwise stated. For topology screening experiment, Tukey's post-hoc test was adopted as the number of groups is more than 5. Statistical significance was set at 0.05 and all statistical analyses were executed by IBM SPSS Statistics 20.0.

Results

Native bNPCs Phenotype

The bovine nucleus pulposus (bNP) tissue were characterized for the native phenotype of bNPCs Routine haematoxylin and eosin staining (data not shown) revealed that the round bNPCs loosely interspersed in the ECM of the native NP tissue with little cell-cell contact. Positive Safranin O staining (data not shown) revealed abundant sulfated GAGs in the native NP, immune-positive staining against COL2 (data not shown) and immuno-negative staining against COL1 (data not shown) revealed the physiological phenotype of bNPCs. The selected bNPC phenotype markers were all positively stained by IF staining of the cryo sections of native bNP tissues. Specifically, the bNPCs sparsely housed in the bNP tissues were positively-stained with phenotype markers including COL2 (data not shown), KRT8 (and SNAP25 (data not shown). It was obvious that COL2 were present in both the native ECM and the intracellular region. Besides, apart from the round bNPC morphology as illustrated in staining of all phenotype markers, the actin filaments also showed the typical phenotype of bNPCs with a thin ring of cortical F-actin network at the cell periphery (data not shown).

Dedifferentiated Phenotype of bNPCs Upon Monolayer Subcultures

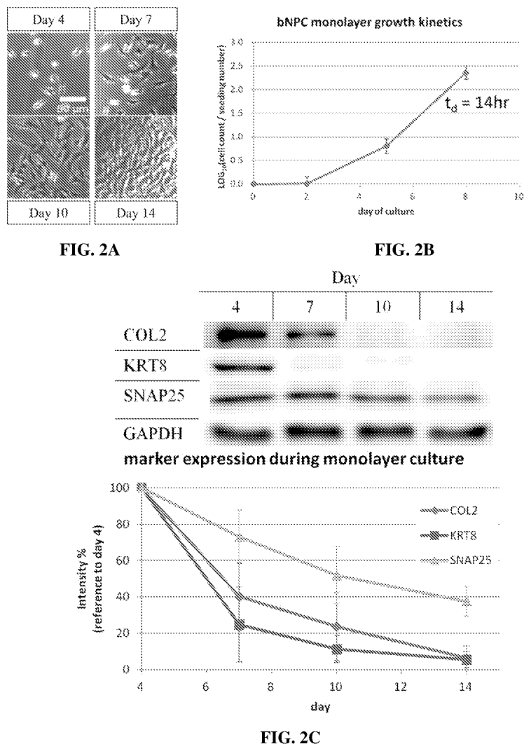

Monolayer cultured bNPCs were characterized for their phenotype to verify whether these cells dedifferentiate upon monolayer cultures and determine the time frame for evaluation of the NPC phenotype. The NPC phenotype markers being investigated were selected based on their high expression level relative to that of similar cell types (articular cartilage (AC) cells or annulus fibrosus (AF) cells). As a result, whether the 2D cultured bNPCs indeed de-differentiated and the reduced expression of these bNPCs phenotype markers upon de-differentiation have to be verified. Specifically, in FIG. 2A monolayer cultured bNPCs gradually changed their morphology from a polygonal shape (day 4) to an elongated shape (day 10) while they proliferated over time from sub-confluence (day 7) to confluence (day 14). FIG. 2B shows the growth kinetics of bNPCs in monolayer cultures and the proliferation rate increased exponentially and their doubling time was less than 14 h between day 5 and day 8. This rapid growth is non-physiological and differs from the slow turn-over rate of NPCs in native tissues. The IF staining of the selected NPC phenotype markers (COL2, KRT8 and SNAP25) (data not shown) demonstrated a significantly decreased number of cells positively expressing these markers and a decrease in the intensity of the fluorescence signals of these markers from day 4 to day 7 (data not shown). Semi-quantitative analysis of Western blot (FIG. 2C) of the phenotype markers of bNPCs including Collagen type 2 in blue channel, keratin 8 in red channel and snap25 in green channel in the total cell lysate of the 2D cultured bNPCs verified the fact that these phenotype marker proteins significantly reduced in expression during the 14-day duration of 2D culture. One-way ANOVA with Bonferroni's tests showed that the expressions of all phenotype protein markers on day 4 were significantly higher than that of later time points, including COL2 (with day 7, p=0.004; with day 11&14, p<0.001), keratin 8 (with day 7,11&14, p<0.001) and SNAP25 (with day 7,11&14, p<0.001). These results confirmed that bNPCs were de-differentiated upon 2D monolayer cultures. Moreover, the specific bNPC phenotype markers (keratin 8 and SNAP25) in addition to the traditional NPC marker (COL2) were sensitive to the dedifferentiation process, which took place rapidly within the first week of monolayer cultures. Collectively, these results verified that bNPCs in monolayer cultures indeed de-differentiated and reduced the expression of the phenotype markers including COL2, keratin 8 and SNAP25. The expression level of these markers at 1 week was therefore chosen as the readout for the phenotype maintenance of bNPCs in the subsequent cell niche screening.

Ultrastructural Characterization of 3D Protein Microstructures with Specific Topological Features

The multiphoton microfabrication technology has an excellent spatial control of the microstructures created and hence was used to fabricate protein cell niche with a wide range of topological features. In brief, a custom-made Matlab program was written for fabrication of different topologies. Common CAD software output (.stl), simple image files and self-written codes are supported. The .stl files drawn by SolidWorks was imported for voxelization, slicing, organizing and file generation. A set of ROI overlay files for the multiphoton confocal microscopes was generated. The complex topology was then fabricated with multiple ROIs at different z-levels.

The ultrastructure of the fabricated protein microstructures with different topological features were examined by scanning electron microscopy (SEM). FIG. 3A shows the SEM images of BSA flat matrix at low and high magnification. The high magnification image shows the BSA microstructure, which were formed by solidifying BSA macromolecules in the solution into submicron protein aggregates, formed upon multiphoton photochemical crosslinking. FIG. 3B shows the SEM images of protein cell niche with different topological features with top and tilted views, as well as magnified views in the insets. The names (abbreviations) of the 9 topological features are BSA flat matrix (BSA/FM) (FIG. 3A), micro-pillar array (MPA) (FIG. 3B(i)), fiber-bead microstructure (FB) (FIG. 3B(ii)), thick grating (TkG) (FIG. 3B(iii)), thin grating (TnG) (FIG. 3b(iv)), parallel grating hierarchy (GHpl) (FIG. 3B(v)), perpendicular grating hierarchy (GHpp) (FIG. 3B(vi)), convex (Cv) (FIG. 3B(vii)) and concave (Cc) (FIG. 3B(viii)).

Low Stiffness Favors the Expression of bNPC Phenotypic Markers

As first line screening, bNPCs were cultured on protein cell niche with a spectrum of individual niche factors including three levels (low, intermediate, high) of two types of mechanical properties (elastic modulus, stiffness), 9 topological features (flat matrix, micro-pillar array, fiber-bead hierarchy, thick grating, thin grating, parallel grating hierarchy, perpendicular grating hierarchy, convex and concave) and three levels (low, intermediate, high) of four types of ECM (fibronectin, laminin, vitronectin, fibrinogen).

Previous studies demonstrated that the mechanical properties (modulus and stiffness) were able to be quantitatively controlled by manipulating a series of fabrication parameters including but not limited to the laser power and the number of scanning cycles. The fabrication parameters of the protein cell niches and the schematic diagram showing the fabrication of these protein microstructures with different levels of modulus and stiffness parameters are shown in FIGS. 3C and D, respectively. Phenotype marker expression of bNPCs cultured on flat protein matrix niche with different levels of elastic modulus showed the IF staining of the bNPC phenotype markers counter-stained with F-actin to show the cytoskeleton and with DAPI to show the nuclei. Low modulus showed slight increase in the intensity of the IF staining of KRT8 and SNAP25 (data not shown). FIG. 3E shows the clustered bar charts on the intensities of the IF staining in different groups for all three selected bNPC phenotype markers upon culturing of bNPCs on protein cell niche with different elastic modulus. The red dotted line represents the expression level of the particular phenotype markers of bNPCs cultured on flat BSA micro-matrices as the reference group. There was ˜25% of difference in the intensities of the COL2 phenotype marker expression with the reference line. One-way ANOVA with Bonferroni's post-hoc tests showed that there was statistical significance between the low and the medium levels in COL2 (p<0.01) and between the low and the medium levels in Keratin 8 (p<0.01), but not in SNAP25. FIG. 3F shows the clustered bar chart of the IF staining intensity of the bNPC phenotype markers normalized by that of the reference group (bNPCs cultured on flat BSA matrices) upon culturing of bNPCs on protein cell niche with different stiffness. All experimental groups enhanced the phenotype marker expression, for up to 75%, as compared to the reference group (red dotted line), suggesting that the micro-pillar array may be a favorable topological feature for bNPC phenotype maintenance. Comparison among different levels of stiffness of the micro-pillar arrays, it is obvious that the low stiffness group favors higher expression level of the phenotype markers including COL2 (1st cluster) and KRT8 (2nd cluster), with statistical significance (one-way ANOVA with Bonferroni's post-hoc tests, p<0.05 and p<0.01, respectively). There was no statistical significance in the expression level of the phenotype marker SNAP25 (3rd cluster) between any two stiffness levels (p>0.05) but a statistically significant trend for SNAP25 was found (polynomial contrast, linear, p<0.05), suggesting that there was a significant increasing trend of SNAP25 expression as the stiffness decreases. In summary, protein cell niche with low stiffness represents a favored mechanical property for bNPC phenotype maintenance.

Topological Features Namely Micro-Pillar Arrays and Fiber-Beads Hierarchy Favor bNPCs Phenotype Maintenance

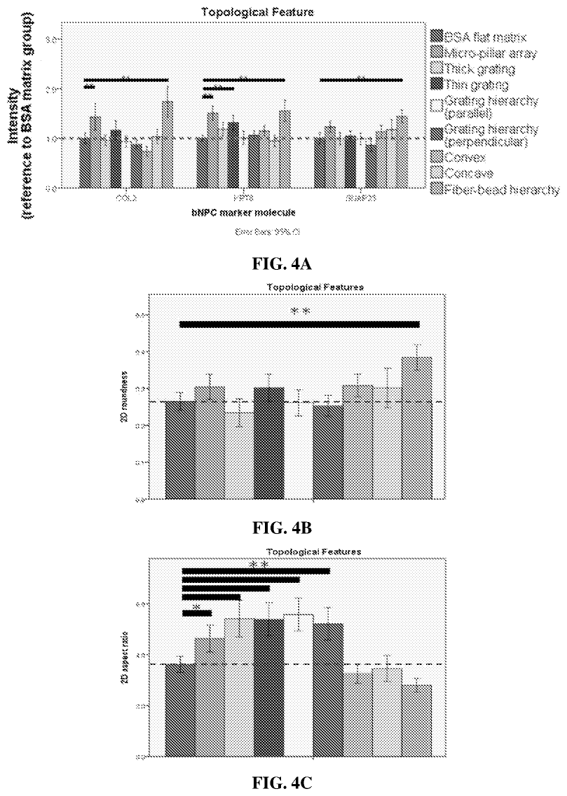

Representative images of the IF staining of the selected phenotype markers in bNPCs cultured on 9 selected topological features were taken (data not shown). Among all groups, the bio-inspired topological feature fiber-bead microstructure (FB) showed the highest IF staining intensity of COL2, KRT8 (and SNAP25. Moreover, similar to other grating groups, bNPCs cultured on the topological feature thin grating showed relatively low staining intensity of COL2, and SNAP25 and an elongated and bipolar morphology with strong alignment along the direction of the gratings. FIG. 4A shows the clustered bar chart on the semi-quantitative measurement of the IF staining intensity of the bNPC phenotype markers, normalized by that of the reference group (bNPCs on BSA flat matrix) and hence the red dotted reference line represents the baseline intensity level at unity. One-way ANOVA with Tukey's post-hoc test showed that the MPA group (green bar) was statistically significantly higher from the FM reference group (dark blue bar) for both COL2 and KRT8 (both p<0.01), the FB group (sky blue bar) showed significantly higher intensity for all three phenotype markers (COL2, KRT8, SNAP25; all p<0.01) while the thin grating (TnG) group (purple bar) showed significant increase for KRT8 (p<0.01). The differences between the reference line and the MPA group or the FB group ranged from 50 to 100%, suggesting that these two topological features critically favor the bNPC phenotype maintenance. In addition to the positive expression of the phenotype markers, the native bNPC phenotype was also characterized by their round morphology with low aspect ratio. FIGS. 4B and 4C showed the clustered bar charts on quantitative image analysis on the roundness and the aspect ratio, respectively, of the bNPCs cultured on protein cell niche with different topological features in the MIP images. The FB topological feature is the only group that showed a statistically significantly increase in the roundness of the bNPCs as compared with the FM reference group (p<0.01) (FIG. 4B). The FB topological feature group also showed the lowest aspect ratio among all groups but the difference was statistically insignificant (FIG. 4C). Moreover, all grating structures (thick and thin, simple and hierarchical) and the micropillar array (MPA) showed significantly higher aspect ratio than the reference group (p<0.05 for MPA and p<0.01 for all grating groups). In summary, protein cell niches with MPA or FB topological features favored maintenance of bNPC phenotype markers, particularly in terms of increased expression of COL2 and KRT8 phenotype markers, and exhibition of a round morphology as that in native NP tissue.

ECM Laminin and Vitronectin Favor bNPC Phenotype Maintenance

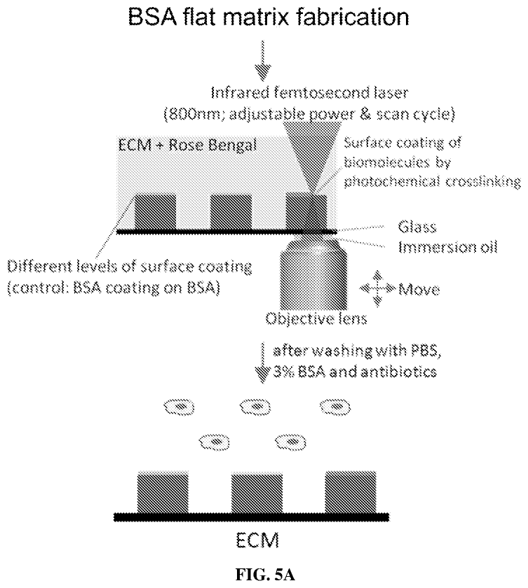

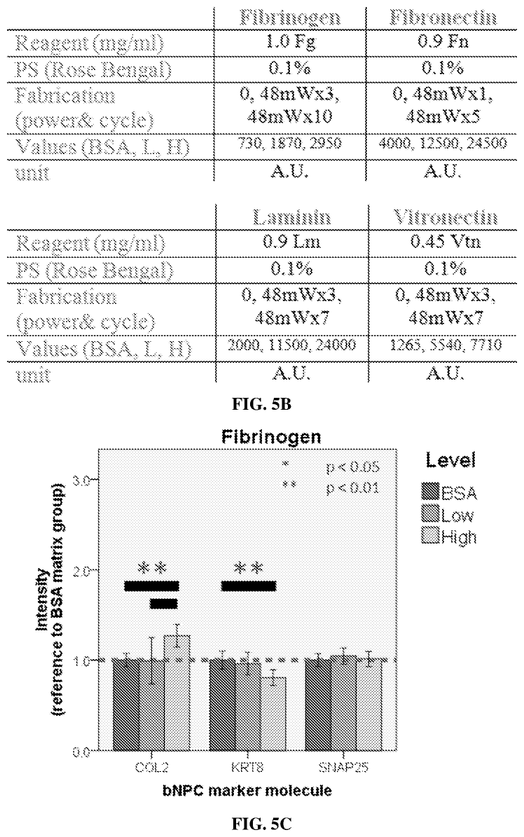

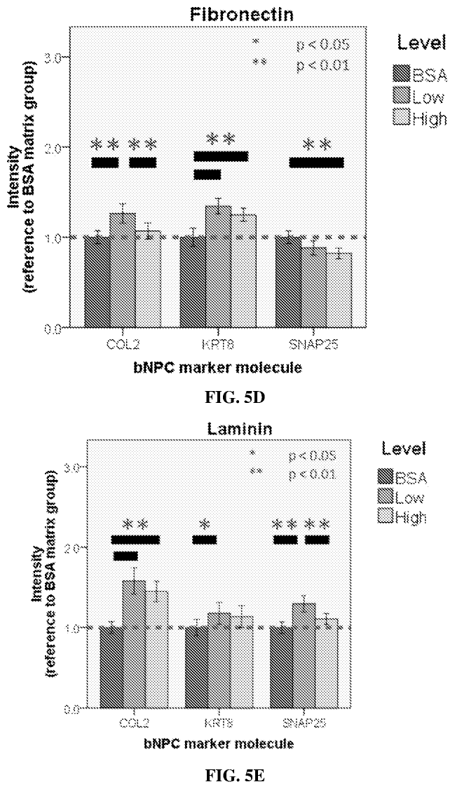

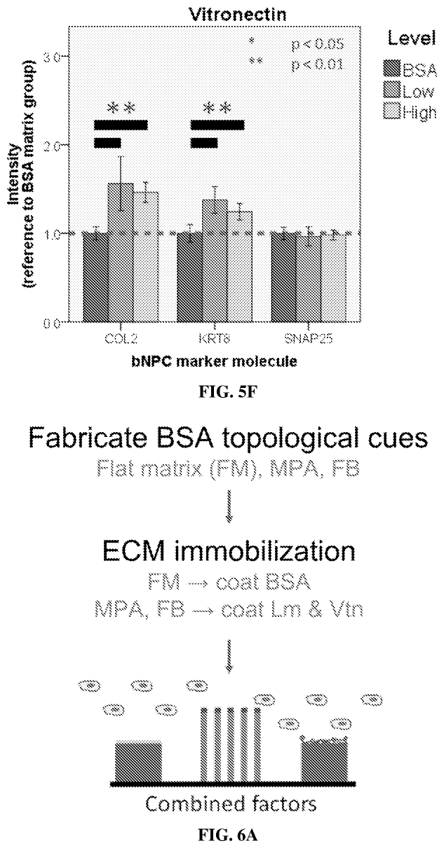

Apart from mechanical and topological factors, biochemical factors such as the type and the density of the local ECM also present key cell niche factors. Flat micro-matrix (FM) microstructures were functionalized or surface-coated with different ECM components (fibrinogen (Fg), fibronectin (Fn), laminin (Lm), and vitronectin (Vtn)), each with either low or high local density levels by the same MMM technology to evaluate the effects of the ECM cell niche factors on bNPC phenotype maintenance. FIGS. 5A and 5B shows the fabrication set-up and the parameters for the FM micropatterns used for ECM screening. Four types of ECMs were surface-coated via the MMM technology to the BSA flat micro-matrices with either low or high local density controlled by adjusting the laser power and the scan cycle. The control group represents a layer of BSA, rather than ECM, surface coated to the BSA flat micro-matrices.

As the first line screening for the ECM cell niche factors, FIG. 5C-F shows the IF staining and its semi-quantitative analysis of the expression of the phenotype markers in bNPCs cultured on protein cell niche substrates with ECM fibrinogen (Fg) (C), fibronectin (Fn) (D), laminin (Lm) (E), and vitronectin (Vtn) (F), respectively. IF staining of the bNPCs cultured on FM cell niches without ECM coating, and with either low or high local density of Fg (C), Fn (D), Lm (E and Vtn (F) was conducted. FIGS. 5C-F show the clustered bar charts of the IF staining intensities of the phenotype markers of bNPCs cultured on the cell niches with different types and levels of ECM. The red dotted reference line was the intensity levels for bNPCs cultured on BSA-coated BSA flat micro-matrices. The asterisks in the charts represent the statistical significance between groups using one-way ANOVA with Bonferroni's post-hoc test. Specifically, bNPCs showed a small but significant increase in COL2 expression in the high-level Fg group as compared with the low-level Fg group and the reference groups (p<0.01, value <1.25 times of ref group) (FIG. 5C). Besides, the high Fg density group also reduced the KRT8 expression significantly (p<0.01). FIG. 5D shows that the low-level Fn group significantly increased the COL2 and KRT8 staining intensity as compared to the reference BSA group (p<0.01) while the high-level Fn group significantly increased the COL2 staining intensity. However, the SNAP25 expression decreased as the Fn level increased (p<0.01). FIG. 5E shows that the expression of all three bNPC phenotype markers (COL2, KRT 8 and SNAP25) was significantly increased up to 1.2-1.5 fold of the reference group (p<0.01 for COL2 and SNAP25, p<0.05 for KRT8) in the low-level Lm group. The high-level Lm group showed less increase in the phenotype marker COL2 expression than the low-level group, but still significantly greater than the reference group for COL2 (p<0.01). FIG. 5F shows that both the low-level and the high-level Vtn groups expressed significantly more COL2 and KRT8 up to 1.5 fold of the reference group (one-way ANOVA with Bonferroni's post-hoc test, all p<0.01). In summary, laminin and vitronectin were shortlisted as the ECM niche factors enhancing the phenotype maintenance of bNPC through increased expression of the bNPC phenotype markers (COL2, KRT8 and SNAP25).

Combinatory Cell Niche Integrating Mechanical, Topological and ECM Factors Optimally Maintained bNPC Phenotype

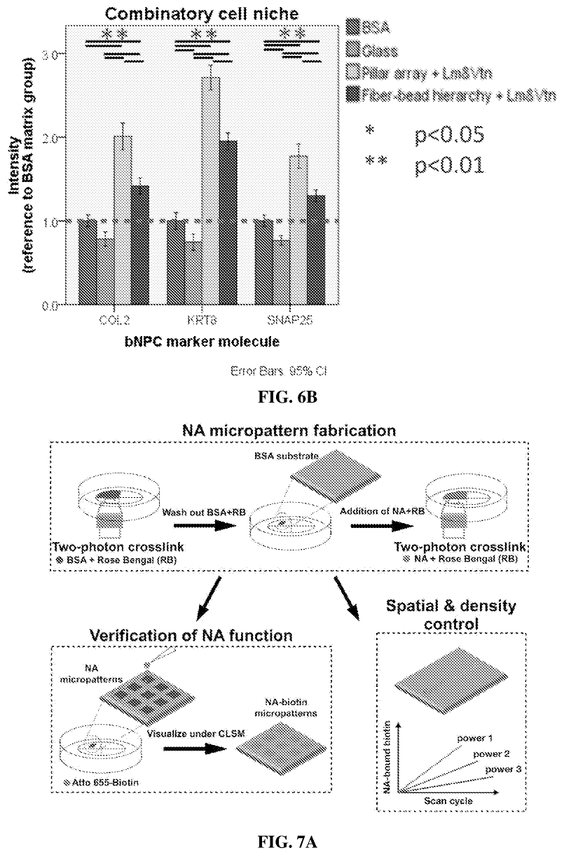

Individual cell niche factors shortlisted from the first line screening (FIGS. 3-5) were used to design combinatory cell niches integrating mechanical (low stiffness), topological (micro-pillar array and fiber-beads) and ECM (laminin and vitronectin) niche factors. Two types of combinatory cell niches, namely micropillar arrays surface-coated with both laminin and vitronectin (MPA+Lm&Vtn), and fiber-beads arrays surface-coated with both laminin and vitronectin (FB+Lm&Vtn) were fabricated by the MMM technology. The phenotype markers of bNPCs cultured on these combinatory cell niches were compared with that of two control groups (BSA-coated flat micro-matrix (BSA) and glass bottomed culture dish). FIG. 6A shows the schematic diagram of the experimental procedures for the second line screening. Both the laminin and vitronectin were successfully photochemically crosslinked onto the same complex fiber-bead topological micro-structures (data not shown). It was noticed that laminin was mainly immobilized on the edges of the BSA fiber-beads micro-patterns while vitronectin was immobilized throughout the entire fiber-beads micro-patterns. This may be due to the fact that vitronectin has a much smaller molecular size (75 kDa) than laminin (900 kDa). Therefore, vitronectin could easily diffuse into the tiny pores of BSA micropatterns and some could be crosslinked or immobilized inside the protein microstructure while laminin could not. Representative maximum intensity projection images of the IF staining of the selected phenotype markers (COL2, KRT8 and SNAP25) expressed by bNPCs cultured on the protein cell niches, counter-stained with F-actin and DAPI were obtained (data not shown). The overall staining intensity of all three phenotype markers in green channels, COL2, KRT8 and SNAP25 were much higher in the combinatory cell niches, laminin and vitronectin coated micro-pillar arrays and fiber-beads micro-patterns than the flat protein micro-matrix and the glass substrate (control groups). FIG. 6B shows the clustered bar charts of the quantitative measurement of the phenotype marker expression levels in different groups. The red dotted reference line shows the level of the BSA-coated flat micro-matrix control group (BSA). The staining intensities of all three phenotype markers were significantly increased for up to 3 folds in the combinatory cell niche groups as compared with the control groups (FIG. 6B) (one-way ANOVA with Bonferroni's post hoc-test, p<0.001). Both combinatory cell niches significantly enhanced the phenotype maintenance of bNPCs and the micro-pillar arrays surface-coated with laminin and vitronectin (yellow bars) showed the best outcomes.

Discussion

bNPCs are More Sensitive to Stiffness than Elastic Modulus in Phenotype Maintenance

The native matrix environment of bNPCs is soft and gel-like. The mechanical cell niche is frequently referred to two related but distinct mechanical properties namely stiffness, which is an extrinsic property of a structure and is dependent on both material strength and geometry, and elastic modulus, which is intrinsic material property that is geometry-independent. The elastic modulus of natural NP has been measured in different species with a wide range from a few kPa to hundreds of kPa. Multiphoton micro-fabricated BSA protein micro-structures used in the current study had an intermediate elastic modulus ranging from 15 to 45 kPa, which is within the reported range. bNPCs only showed a few changes in their phenotype marker expression in response to the change in elastic modulus. On the other hand, bNPCs showed a more consistent change in their phenotype marker expression in response to change in stiffness, that a lower stiffness or a softer protein matrix seems to favor better phenotype maintenance, particularly in COL2 and KRT8. COL2, a traditional chondrocytic phenotype marker used in both chondrocytes and NPCs, has been demonstrated to reduce its level of expression in porcine chondrocytes upon increase in elastic modulus of a fibrinogen-functionalized hydrogel, in the presence of the chondrogenic medium. KRT 8 has been reported to be a relatively more NPC-specific phenotype marker than the traditional chondrocytic phenotype marker COL2. It is known that KRT8 molecules work with KRT18 in epithelial cells to form intermediate filament to maintain the cell integrity, control cell differentiation and other functions. Increasing expression of KRT8 in bNPCs against the decreasing stiffness of the protein micropillar cell niche suggests the mechano-sensing ability of the bNPCs. The MMM platform is able to fabricate 3D protein microstructures such as micropillar arrays with a wide range of aspect ratios and hence magnitudes of difference in stiffness. Take a micropillar with 1 μm diameter for example, the stiffness of the protein micropillar arrays fabricated can be easily manipulated to vary within a large range (17.3-1104 pN/μm) by adjusting the height of the micropillars (4-16 μm). One implication is that the geometry-dependent stiffness, or more generally, the softness or rigidity of the cell niche structures, may presents an important design parameter for bio-inspired culture substrates or biomimetic scaffolds with the intention to maintain cellular phenotypes.

Micropillar Array and Fiber-Beads Topographies Better Maintained the bNPC Phenotype

Topological features are determined by both the shape and length scales of its constituents. In the current study, a selected spectrum of topological features including the control flat matrix, simple gratings, hierarchical grating, convex and concave structures, micropillar arrays and arbitrarily-defined irregular fiber-bead microstructures for bNPC cultures with phenotype maintenance. Bench marking with the bNPC phenotype in native NP tissues, round morphology and high level of expression of all three selected markers. In the first line screening, grating features, no matter if it were thin or thick, simple or hierarchical, induced an aligned and bipolar phenotype of bNPCs, with low expression of the three phenotype markers, suggesting that these topological features cannot maintain the native phenotype of bNPCs in cultures. bNPCs on flat matrix, convex and concave protein microstructures did not show significant difference in cell morphology, aspect ratio and phenotype marker expression. Interestingly, bNPCs cultured on the two complex topological micropatterns, namely the fiber-beads (FB) and the micropillar arrays (MPA) best maintained their phenotype as shown by consistently high expression of the three chosen phenotype markers (COL2, KRT8 and SNAP25). Additionally, the bNPCs on FB structure also maintain a relatively round morphology. This may be due to the bio-mimetic and bio-inspired nature of these two topological features. The FB design was inspired by the ultra-structure of the native NP collagenous meshwork and space-filling beads-like ground substances such as glycosaminoglycans (GAGs) at submicron scale by SEM. The other biomimetic topological feature MPA was also effective in phenotype maintenance of bNPC. In a previous study, fibroblasts growing on top of micropillar array attached to the microstructure by gripping the micropillar tips and showed a more 3D-like morphology than those cultured as monolayers. These adhesion-like structures around the micropillar headpieces resemble the point contact or anchorage between a cell and its surrounding natural ECM fibrous meshwork. The enhanced phenotype maintenance in bNPCs by these two topological cell niche factors highlight the importance of bio-inspired or bio-mimetic design in reconstituting physiologically relevant culture substrates.

Vitronectin and Laminin Consistently Maintained Better bNPC Phenotype

The current study shows that laminin and vitronectin preferably enhanced the expression of selected bNPC phenotype markers singly during the first stage of screening and in combination in the second stage of screening. Moreover, in the second line screening, the combination of laminin and vitronectin showed significantly augmented phenotype maintenance than their individual effect, suggesting that reconstituting the complex matrix niche in the native NP tissue is important for the phenotype maintenance of its resident cells. In the current study, only 4 ECM factors were selected for screening, more complex cell niche with additional matrix factors, such as proteoglycan and collagen, as identified from NPC transcriptome and proteome can be incorporated for further optimization of cell niche for NPC phenotype maintenance.

Optimal Cell Niche Substrates for bNPC Culture with Phenotype Maintenance

The MMM technology on one hand enables decoupling of individually controlled cell niche factors, but on the other hand, is able to fabricate complex combinatory cell niche with multiple defined cell niche factors, therefore allowing us to fabricate combinatory cell niche in the second line screening by combining the cell niche factors shortlisted from the first line screening, including a low stiffness, the micropillar array and fiber-beads topologies, and the vitronectin and laminin ECM, resulted in additional upregulation of the expression of the all three phenotype markers of bNPCs for up to 3 fold, as compared with the control groups (glass bottomed dish and flat BSA matrix). This result demonstrates the effectiveness of this protein multiplex cell niche screening platform and in shortlisting individual niche factors and verifying the combinatory effects of multiple cell niche factors in optimizing the bNPC phenotype maintenance. Among all the cell niche factors being investigated, the micropillar arrays functionalized with laminin and vitronectin ECM was found to be the optimal cell niche environment for bNPC phenotype maintenance in cultures. The two combinatory cell niche designs can be used as biomimetic culture substrates for phenotype-maintaining bNPC cell cultures, physiologically-relevant drug testing, and rationalized scaffold design.

Multiplex Cell Niche Screening Strategy

This study reports an effective two-stage approach to screen for the optimal cell-niche for phenotype maintenance in an example cell type bNPC. The selection of relevant readouts for phenotype maintenance deserves paramount attention in screening studies. The markers must be sensitive to the biological phenomenon being studied, in this case phenotype maintenance of bNPCs. The phenotype markers of bNPCs were selected from the literatures according to their high-level expression when compared to the internal reference intervertebral disc cells (annulus fibrosus cells) and the external reference chondrocytic cells (articular chondrocytes). Moreover, the sensitivity of these phenotype markers (COL2, KRT8, SNAP25) during processes such as dedifferentiation upon monolayer cultures has been validated. Multiple phenotype markers (three in this study) have been selected as the readout so as to increase the reliability of the screening results as the probability to have multiple false-positive results simultaneously should be extremely small. Given the huge number of experimental conditions, both in the type and level of the multiple cell niche factors, the total number of experimental conditions and their combinations should increase exponentially and tremendously. As a result, “full-spectrum” screening using all possible cell niche factors is impractical. Instead, the disclosed two-stage approach, first shortlists the best performing individual cell niche factors and secondly verifies the optimal phenotype maintenance of the combinatory cell niches, is cost-effective in engineering biomimetic complex cell niche with optimal performance.

### 2. Multiphoton Based, Spatially and Quantitatively Controlled Micropatterns of Soluble Factor Gradients

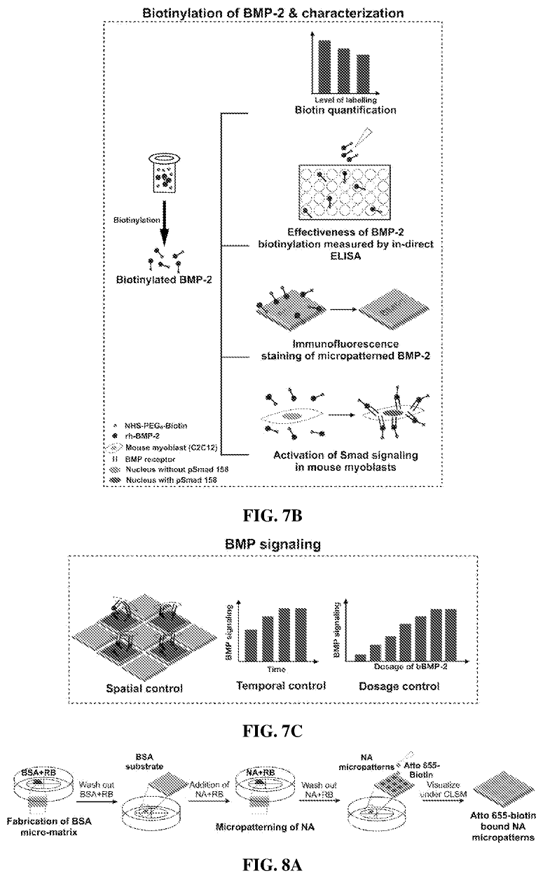

The MMM technology is utilized to micropattern a model soluble cell niche, bone morphogenetic protein-2 (BMP-2), with spatial and quantitative control, and sustained bioactivity, in a three-step process (FIG. 7). First, bovine serum albumin (BSA) micro-structure, flat matrix in this case, was fabricated on the glass surface via a rose Bengal-mediated two-photon photochemical crosslinking, followed by micropatterning a thin layer of neutravidin (NA) on its surface using the same photochemical crosslinking process. Second, BMP-2 was biotinylated and the biotinylated BMP-2 was characterized. Finally, the optimally biotinylated BMP-2 is micropatterned through functional binding onto the pre-micropatterned NA. Notably, the bound BMP-2 (bBMP-2) not only retained its full bioactivity through activation of the downstream Smad signaling in mouse myoblasts (C2C12), but also exhibited a more sustained and a higher level of Smad signaling than the free BMP-2 (fBMP-2) group, suggesting an even more efficient soluble factor micropatterning platform. With this platform, BMP-2 and its downstream Smad signaling can be micropatterned with quantitative and spatial control. This disclosure reports a surface modification-free, non-contact and mask-free micropatterning approach, providing a convenient, robust and universal all-in-one tool to reconstitute soluble cell niches in vitro for pleiotropic applications such as development of biomimetic soluble cell niche biochips for signal transduction study and drug screening.

Two-Photon Fabrication System

A confocal laser scanning microscope (CLSM) (Carl-Zeiss 710, Jena, Thuringia, Germany) equipped with a mode-locked Ti:sapphire femtosecond near infrared (NIR) laser (Coherent, Santa Clara, CA, USA) was used for fabrication. The NIR laser with maximal output at a wavelength of 800 nm, emitting through a 40× oil lens with a numerical aperture (NA) of 1.3 (EC Plan-Neofluar 40×/1.30 Oil DIC M27) are used. The default ZEN 2010 software is used to control the fabrication. A power meter (Coherent) is used to measure the NIR laser output power before each round of fabrication.

Fabrication of Bovine Serum Albumin (BSA) Substrates and Micropatterning of Neutravidin (NA)

Silicone micro-inserts (ibidi, #80409, Munich, Bavaria, Germany) sticking onto a 35 mm glass-bottomed dish (MatTek, P35G-1.5-14-C, Ashland, MA, USA) are used as the containers for fabrication. An aliquot of 10 μl bulk protein solution consisting of the substrate protein bovine serum albumin (BSA) at 300 mg ml⁻¹ (#A4378, Sigma-Aldrich, St. Louis, MO, USA) and the photosensitizer rose Bengal (RB) at 0.1% w/v (#330000, Sigma-Aldrich) is loaded into the micro-insert well followed by He—Ne laser scanning to locate the solid-aqueous interface between the glass bottom and the bulk protein solution. The position of interface is set as zero. The BSA protein substrate in the form of flat micro-matrix with dimension of 101×101×1 μm (length×width×height) is first fabricated by z-stack scanning of the 800 nm two-photon laser through the bulk protein substrate solution from 1 μm below to 1 μm above the zero position. The fabrication parameters are as follows: (1) z-stack interval is kept at 0.5 μm; (2) scanning power, speed and scanning cycle are set to 180 mW, 1.27 μs, and 1, respectively; (3) scanning zoom is 2.1 to give a scanning area of 101×101 μm; (4) frame size is kept at 512×512 to give a pixel size of 0.2×0.2 μm. After fabrication of the BSA micro-matrix substrates, the excess bulk protein solution is removed, followed by thorough rinsing with phosphate buffered saline (PBS, 1×) (#18912014, Thermo Scientific, Rockford, IL, USA). An aliquot of 5 μl of NA solution comprising the molecule being crosslinked neutravidin (NA) (#31000, Thermo Scientific) and the photosensitizer RB (0.1% w/v) is applied to the same well. After setting the solid-aqueous interface at the pre-formed BSA substrate, multiphoton laser scanning from 0.5 μm below the interface with a step size of 1.5 μm is used to fabricate the NA micropattern in register with a pre-designed region of interest (ROI) as the micropattern design. The unreacted NA solution is then discarded and the well is thoroughly rinsed with PBS (1×) before sterilizing the resultant NA micropatterned protein micro-matrices by immersing in a PBS solution containing 4% v/v of Antibiotic-Antimycotic (100×) (#15240096, Thermo Scientific) at 4° C. for 24 hours. In order to reduce any non-specific binding of molecules to NA patterns, the NA micropatterned BSA micro-matrices are blocked with 5% BSA solution at room temperature for overnight before subsequent experiments.

Different reagent and laser fabrication parameters are varied to control the local crosslinking density of the NA micropatterns. BSA square micro-matrix (101×101×1 μm (length×width×height)) arrays are fabricated with a fixed laser power at 180 mW. A micropattern of the logo of the University is used as the ROI to illustrate the capability to achieve user-defined micropatterns during the NA micropatterning procedure where the NA molecules are micropatterned on the surface of the BSA micro-matrix by supplementing NA solutions at descending concentrations (9, 5, 2.5, 1.25 and 0 mg ml⁻¹) with a constant laser power of 45 mW and 11 scan cycles, to illustrate the capability to control the local density of the micropatterning materials. Further, in order to demonstrate the capability to control the NA crosslinking density by laser parameters such as scan cycle, a micropattern consisting of a string of letters “Neutravidin” is micropatterned with a series of descending scan cycles (13, 11, 9, 7, 5 and 3 cycles), at a fixed power of 45 mW and a fixed NA concentration of 9 mg ml⁻¹, on the surface of the BSA micro-matrix substrate.

In order to control the amount of the biotin (Atto 655-Biotin) bound to the NA micropatterns, nine NA square micro-matrix measured 20×20 μm are crosslinked on the top of BSA micro-matrix substrate with different fabrication parameters (NA concentration, laser power and scan cycle). Specifically, different concentrations (0, 1.25, 2.5, 5 and 9 mg ml⁻¹) of NA solution, different laser power (27, 36, 45 and 54 mW) and different scan cycle (1, 3, 5, 7, 9, 11, 13 and 15) are used during the micropatterning process. After the micropatterning, the peak fluorescence intensity of the bound Atto 655-Biotin is measured accordingly.

In order to demonstrate simultaneous spatial and quantitative control of BMP-2, a micropattern of a string of letters “BMP2” is used as the ROI, and a different laser scan cycle (descending from 15, 11, 7 to 3 cycle) is used to micropattern each of the letter in the string during the NA micropatterning procedure, at a constant laser power of 45 mW.

For subsequent cellular studies to illustrate the capability of the current micropatterning technology to retain the BMP-2 bioactivity, a 3D micro-well with a wall thickness of 2 μm and a wall height of 7 μm is fabricated along the periphery of the pre-made BSA square micro-matrix (101×101×1 μm), at a constant laser power of 210 mW, to confine the cells within the micro-well for binding and bioactivity assay. Micropatterning of a squared NA micro-island (80×80 μm) on the surface of each BSA micro-matrix substrate within the 3D micro-well is conducted at a constant laser power of 45 mW for 11 scan cycles.

Characterization of NA Micropatterns

To verify the biotin-binding function of the micropatterned NA, Atto 655-Biotin (#06966, Sigma-Aldrich), a fluorophore-tagged biotin, is used. In brief, 5% BSA-blocked NA micropatterns are incubated with Atto 655-Biotin PBS solution (1 μM) in darkness at room temperature for 1 hour, and the fluorescence signal is measured using the CLSM (Carl-Zeiss 710) with a He—Ne laser and a 40× lens at an excitation wavelength of 633 nm and a detection wavelength range of 644-759 nm, after thorough rinsing of the Atto 655-Biotin PBS solution with PBS (1×) in excess for five times (5 minutes each). The relative fluorescence intensity of NA micropatterns bound with Atto 655-Biotin is analyzed by an Image J software (1.51s version, National Institutes of Health (NIH), Bethesda, MD, USA). Specifically, the “‘Plot Z-axis Profile’” function is selected to measure the fluorescence intensity of the z-stack images across the z-axis with a thickness of 4 μm below and above the NA micropattern, giving rise to a Gaussian distribution curve of fluorescence of the Atto 655-Biotin. The “ROI manager” function is used to select the region to be analyzed, both the NA micropatterns and the BSA micro-matrix substrate. The mean value of the Atto 655-Biotin fluorescence intensity of each plane of the z-stack is recorded and the relative mean fluorescence intensity of the Atto 655-Biotin signal on the NA micropatterns is obtained by subtracting the background signal of the BSA micro-matrix substrate from that of the NA micropatterns. The peak fluorescence intensity of NA micropattern-bound Atto 655-Biotin is the amplitude value derived from Gaussian non-linear curve fitting of the relative mean fluorescence intensity of the Atto 655-Biotin obtained above.

Additionally, the biotin binding capacity of the NA micropatterns fabricated with different laser power and scan cycle is calculated by measuring the fluorescence intensity of Atto 655-Biotin bound to the NA micropatterns under different conditions against a standard curve generated by binding Atto 655-Biotin of known concentrations (0, 2.5, 5, 20, 50, 100, 250, 500, 600, 700 and 1000 nM) to NA micropatterns fabricated at constant fabrication condition (laser power: 54 mW; scan cycle: 11; NA concentration: 9 mg ml⁻¹).

Biotinylation of BMP-2

In order to micropattern BMP-2 via avidin-biotin interactions, BMP-2 is chemically conjugated with biotin using a protocol modified from previously reported methods. Briefly, 10 μg of rh-BMP-2 (#120-02C, PeproTech, Rocky Hill, NJ, USA) is reconstituted with 100 μl of sterile ultrapure water (Direct-Q® 3 UV Water Purification System, Merck Millipore, Burlington, MA, USA), followed by PBS (#28372, Thermo Scientific) buffer exchange via a Slide-A-Lyzer MINI Dialysis Unit (10K MWCO, #69570, Thermo Scientific) at room temperature for 30 minutes. Subsequently, different volume of NHS-PEG₄-Biotin (#21329, Thermo Scientific) water solution (1 mM) is mixed with a BMP-2 PBS solution at different molar ratios (NHS-PEG4-Biotin to BMP-2: 2:1, 5:1, 10:1, 20:1 and 40:1) and reacted at room temperature for 1 hour with regular agitation. The unreacted NHS-PEG₄-Biotin and the released N-Hydroxysuccinimide (NHS) in the process of reaction are removed by dialysis at room temperature for 30 minutes and the remaining solution, containing the biotinylated BMP-2 in the form of BMP-2-PEG4-Biotin (bBMP-2), is ready for subsequent experiments.

Characterization of Biotinylated BMP-2 (bBMP-2)

The success of biotinylation of BMP-2 is verified by measurement of both the level of labeling (LOL) of biotin onto BMP-2 and the ability of the biotinylated BMP-2 (bBMP-2) in binding the commercialized NA-coated plastic surface. Firstly, the level of labeling (LOL) of biotin onto BMP-2 is measured by a Pierce™ Fluorescence Biotin Quantitation Kit (#46610, Thermo Scientific) according to the manufacturer's instructions. In brief, the bBMP-2 obtained from the biotinylation process under different reacting conditions (i.e., molar ratios of NHS-PEG₄-Biotin to BMP-2=2:1, 5:1, 10:1, 20:1 and 40:1) is incubated with the Dylight Reporter Working Reagent (volume ratio=1:10) in darkness at room temperature for 5 minutes, and fluorescence signal from the mixture is measured by a microplate reader (SpectraMax M2, Molecular Devices, CA, USA) at an excitation wavelength of 494 nm with an emission wavelength of 520 nm, reading against a standard curve generated from the fluorescence intensity of a series of biocytin solutions with known concentrations (0, 5, 10, 20, 40, 60, 80 and 100 pmol/10 μl), so as to determine the amount (picomoles) of biotin molecules present in each unknown bBMP-2 and finally obtain the moles of biotin per mole of bBMP-2 ratio. Secondly, to further verify the ability of biotinylated BMP-2 to bind NA, the NA surface-bound bBMP-2 with different LOL is measured using an indirect enzyme-linked immunosorbent assay (ELISA) performed on Pierce™ NeutrAvidin™ Coated Plate (#15128, Thermo Scientific). In brief, a serial concentration (3.75, 7.5, 15, 30, 60, 80 and 100 ng ml⁻¹) of either unlabeled (free) BMP-2 (fBMP-2), or bBMP-2 with different LOL are added to the plate and incubated at room temperature for 1 hour. Then a mouse monoclonal anti-BMP-2 antibody (ab6285, Abcam, Cambridge, UK) diluted at 1:500 with wash buffer (Tris-buffered saline with BSA (0.1% w/v) and Tween®-20 (0.05% v/v)) is incubated at room temperature for 1 hour after removal of BMP-2 molecules in excess. Then a goat-anti mouse secondary antibody conjugated with HRP (W4021, Promega, Madison, WI, USA), at a 1:2500 dilution is incubated for 1 hour before adding a color development reagent, a mixture of hydrogen peroxide and tetramethylbenzidine (volume ratio=1:1), for incubation in darkness at room temperature for 20 minutes. At the end of the incubation, the reaction is stopped by 2 NH₂SO₄ and the absorbance at 450 nm is measured by a microplate reader (ASYS UVM 340, Biochrom, Cambridge, UK). Apart from the success of biotinylation of BMP-2, it is important to demonstrate that the biotinylated BMP-2 still retains its bioactivity in terms of activating Smad signaling. As a result, various concentrations of either fBMP-2 or bBMP-2 with different LOL is applied to C2C12 cells and pSmad 1/5/8 nuclear accumulation is used as the readout for the bioactivity or bio-functionality of bBMP-2 (details to follow in the subsequent section).

Quantitatively and Spatially Controlled Micropatterning of BMP-2

To demonstrate that the current system is able to micropattern BMP-2 in a quantitatively and spatially controlled manner, four letters “B”, “M”, “P”, and “2” are used as the ROI during NA micropatterning on the surfaces of the pre-made BSA micro-matrix substrate, using different number of scanning cycles, 15, 11, 7 and 3, respectively, at a constant laser power of 45 mW. On the other hand, after blocking the NA micropatterns with a BSA blocking solution (5% w/v), different amounts of fBMP-2 and bBMP-2 (0, 31.25, 62.5, 125 and 250 ng) together with the BSA blocking solution is supplemented to the NA micropatterns to allow for biotin-avidin binding at room temperature for 1 hour, followed by thorough rinsing with PBS (1×) containing Tween®-20 (0.1% v/v) (PBST). After blocking with PBS containing normal serum (5% v/v) and Triton™ X-100 (0.3% v/v) for 30 minutes, a mouse monoclonal anti-BMP-2 primary antibody (ab6285, Abcam) diluted at 1:100 with a dilution buffer (1×PBS containing BSA (1% w/v) and Triton™ X-100 (0.3% v/v)) is supplemented for incubation at room temperature for another 1 hour. An Alexa Fluor® Plus 647 conjugated goat-anti mouse antibody (A21236, Invitrogen), diluted at 1:400, is used as the secondary antibody. Fluorescence signal is obtained by CLSM with He—Ne laser using 40× lens at an excitation wavelength of 633 nm and an emission wavelength range of 638-755 nm for detection. The fluorescence intensity is analyzed by the same method described above.

Cell Culture

A mouse myoblast cell line C2C12 (ATCC®CRL-1772™; American Type Culture Collection (ATCC), Manassas, VA, USA) is used to evaluate the bioactivity of micropatterned BMP-2 owing to its ability to be transdifferentiated from myoblastic to osteoblastic lineage in the presence of functional BMP-2. Cells at P14 are used in the current study. C2C12 cells are routinely cultured in high glucose Dulbecco's modified Eagle's medium (DMEM) (#11965092, Thermo Scientific) containing 10% fetal bovine serum (FBS) (#16000044, Thermo Scientific) and 1% Antibiotic-Antimycotic (100×) (#15240062, Thermo Scientific) at 37° C. and 5% CO 2 for 1-2 days until 70% confluency. To evaluate the bioactivity of bBMP-2, 2×10⁴ cells harvested from the sub-confluent cultures, are plated in a 35 mm glass-bottomed confocal dish and allowed to grow in the above medium for overnight and undergone starvation in DMEM without FBS for 4 hours before BMP signaling evaluation. For the NA-bound BMP signaling evaluation, upon binding of bBMP-2 onto the NA micropatterns, the C2C12 cells harvested from the sub-confluent cultures that are adapted in DMEM containing 2% FBS for overnight are seeded onto bBMP-2 micropatterns at a density of 1000 cells per condition and are allowed to grow in DMEM containing 2% FBS for 4, 24, 48 and 72 hours.

Activated BMP Signaling

It is known that upon BMP-2 binding to its cellular transmembrane receptors, Smad 1/5/8, a transcription factor, would be phosphorylated, denoted as pSmad 1/5/8, which then forms a complex with Co-Smad (Smad 4), translocating from the cytosol to the nucleus to regulate BMP-2 related gene expression. Therefore, the ratio of the fluorescence signal of pSamd 1/5/8 in the nucleus region to the cytosol region, denoted as N/C ratio, is used as the marker of effective activation of BMP-2 signaling, as previously reported, and hence, as an evaluation of the bioactivity of BMP-2 (bBMP-2 or fBMP-2) in the current study. Specifically, to evaluate the bioactivity of the biotinylated BMP-2, immunofluorescence staining of pSmad 1/5/8 in C2C12 cells is performed after starving the cells followed by incubating the cultures with different concentrations of fBMP-2 or bBMP-2 (0, 100, 200 and 1000 ng ml⁻¹) for 30 minutes. The treated cells are fixed with 4% paraformaldehyde (PFA) in darkness for 15 minutes followed by three washes with PBS (5 minutes each). Afterwards, the fixed cells are permeabilized with chilled 100% methanol at −20° C. for 10 minutes and blocked with blocking buffer at room temperature for 1 hour, after which they are incubated with primary antibody phospho-Smad1/Smad5/Smad9 (#13820, Cell Signaling Technology, Danvers, MA, USA) diluted at 1:800 in dilution buffer at 4° C. for overnight. After discarding the unbound primary antibody and thorough rinsing, the secondary antibody conjugated with Alexa Fluor® Plus 647 (A32733, Invitrogen), diluted at 1:400, is added for an incubation in darkness at room temperature for 1.5 hours. Cell nucleus are counterstained with Fluoro-Gel II with DAPI (#17985-50, Electron Microscopy Sciences, Hatfield, PA, USA) at room temperature for 5 minutes. The fluorescence signal of Alexa Fluor® Plus 647 is detected by CLSM with He—Ne laser using a 40× lens at an excitation wavelength of 633 nm and an emission wavelength range of 638-755 nm for detection. DAPI signal is obtained by two-photon excitation at 700 nm and detection at an emission wavelength range of 437-479 nm. To quantify the ratio of pSamd in nucleus to cytosol, the region of nucleus, cytoplasm and background near the cells is firstly defined by “polygon selection” in Image J. The mean fluorescence intensity of each part is derived under “Measure” function. After that, the mean fluorescence intensity of the background is subtracted from those of both nucleus and cytoplasm to obtain the relative fluorescence intensity of the nucleus and the cytosol regions. Finally, the NpSmad/CpSmad ratio is calculated by dividing the relative fluorescence intensity of the nucleus region by that of the cytosol region.

To investigate the retention of the activated BMP signaling in C2C12 cells by either NA-bound bBMP-2 or fBMP-2 in the medium, bBMP-2 (500 ng) is incubated with pre-blocked NA micropatterns as early described (total area is 0.154 mm²) at room temperature for 1 hour followed by thorough rinsing with PBS (1×) for five times (5 minutes each). Then C2C12 cells maintained in low-serum medium are detached and seeded on the NA micropatterns with or without bBMP-2. The extent of nuclear translocation of pSmad 1/5/8 in the treated C2C12 cells is analyzed at 4 hours, 24 hours, 48 hours and 72 hours post-cell seeding as described above. In order to further explore the dose-dependent response of nuclear pSmad 1/5/8 to the NA-bound BMP-2 concentrations, the BMP-2 micropatterns with varying local densities are generated by (1) applying various concentrations of bBMP-2 with different LOL (2 and 4) to NA micropatterns fabricated with constant parameters (laser power: 45 mW; number of scan cycles: 11), or (2) applying a fixed amount of bBMP-2 with a fixed LOL (4) to NA micropatterns with various biotin-binding capacity via varying fabrication parameters (laser power: 36 mW; number of scan cycles: 7 to 15). The response of pSmad 1/5/8 nuclear accumulation to these BMP-2 concentrations engineered by different parameters is observed and analyzed at 24 hours post-cell seeding.

Statistical Analyses

The quantitative data of NA crosslinking efficiency, NA-concentration dependent crosslinking efficiency and sensitivity of NA-biotin binding are presented as mean±SEM; level of labeling (LOL) and fluorescence intensity of pSmad 1/5/8 in nucleus to cytosol (N/C ratio) are presented as mean±SD. Comparison of means among multiple groups is performed by one-way analysis of variances (ANOVA) with Bonferroni's post-hoc tests. The effects of two factors with multiple levels is analyzed by Two-way ANOVA and comparison of means is performed by simple effects analysis with Bonferroni's post-hoc tests. All statistical analyses are carried out using GraphPad Prism 7.0a (GraphPad Software, San Diego, CA, USA). P value <0.05 is considered statistically significant.

Results

Spatially and Quantitatively Controlled Micropatterning of Functional NA by Multiphoton Fabrication

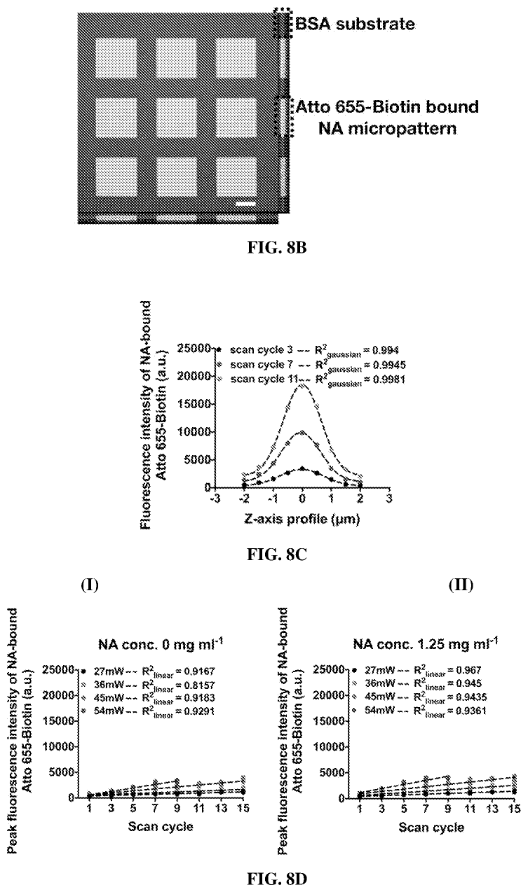

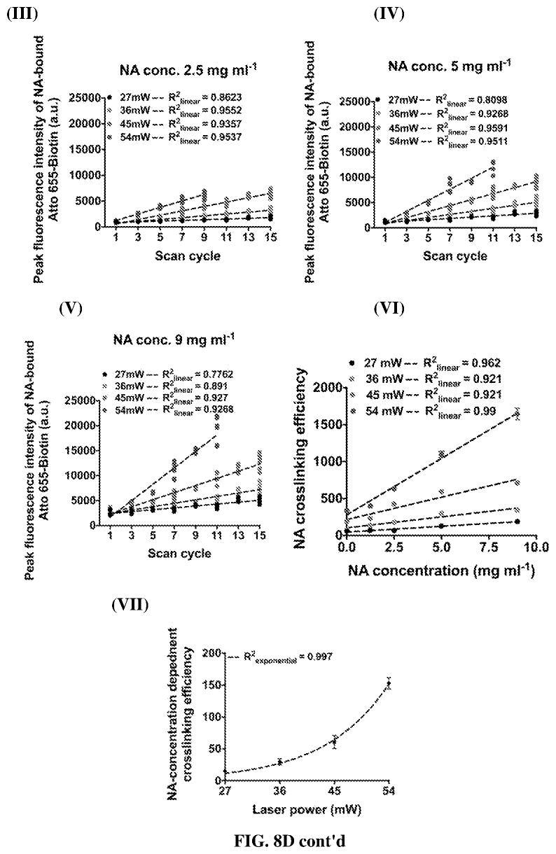

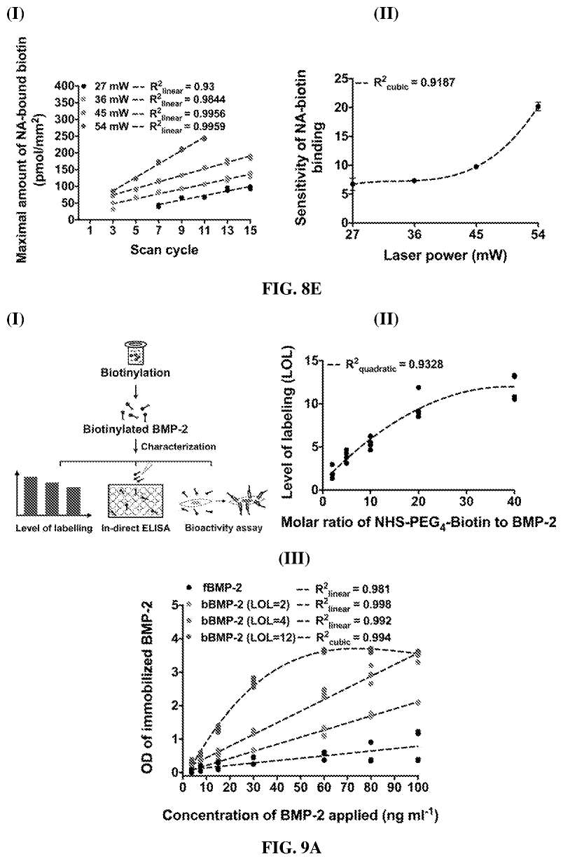

Given the delicate nature of soluble niche factors and the importance of their oriented presentation to effectively trigger downstream signaling, an indirect micropatterning approach, where NA is firstly micropatterned using the MMM technology, before creating BMP-2 micropatterns through functional binding between biotinylated BMP-2 and the micropatterned NA, is developed. First, whether NA can be micropatterned by the multiphoton microfabrication without compromising its functional binding with biotin is investigated. Specifically, a multiphoton laser is used to directly print arbitrary NA micropatterns on prefabricated BSA substrates before evaluates the biotin-binding ability of the NA micropatterns via measuring the fluorescence signal of the fluorophore-conjugated biotin (Atto 655-Biotin) (FIG. 8A). The biotin-binding function of the micropatterned NA is successfully retained after photochemical crosslinking, as demonstrated by the bright fluorescence signal from all NA micropatterns (data not shown and FIG. 8B). The free-form spatial control of the NA micropatterning is demonstrated by the fluorescence images of the HKU logo (data not shown), the “Neutravidin” (data not shown and the square micro-matrix in FIG. 8B. The NA-biotin binding is specific as the negative control showed very low background signal. Moreover, the NA-biotin binding was also highly sensitive as increasing the fabrication parameters, such as the NA concentration results in an increase in the fluorescence intensity of a series of HKU logo-shaped NA micropatterns, and the laser scan cycle results in an increase in the fluorescence intensity of a string of letters “Neutravidin”. To demonstrate the ability of the MMM technology to quantitatively control the local density of the NA and hence the intensity of the fluorescence signal, NA square micro-matrix pattern is created (FIG. 8B by varying the dosage of the laser factors (laser power and scan cycle) and the reagent factor (NA concentration). The value of the fluorescence intensity of the biotin-bound NA micropatterns (squares in FIG. 8B is derived from the peak value of the Gaussian non-linear curve fitting of the relative mean fluorescence intensity in each plane of the z-stack images (FIG. 8C). A significant positive association between the local density of the micropatterned NA and the scan cycle can be obtained when keeping NA concentration and laser power constant (P<0.0001 and R²ₗᵢₙₑₐᵣ>0.77 in all NA concentrations (0, 1.25, 2.5, 5 and 9 mg ml⁻¹) and laser powers (27, 36, 45 and 54 mW)) (FIG. 8D (I)-(V)). The laser scan cycle represents the exposure time to laser pulses, and that directly associates with fluence, which is the optical energy delivered per unit area. In another word, at a particular laser power or NA concentration, the amount of NA photochemically crosslinked in the micropatterns is directly and linearly associated with the amount of laser energy delivered to the constant sized NA micropatterns, demonstrating the controllability of scan cycle over the amount of NA in the micropatterns. Apart from the scan cycle, the laser power and the NA concentration provide additional control over the local density of the micropatterned NA. Specifically, the slope of the linear curves in FIG. 8D (I)-(V) refers to the rate of change in the amount of NA crosslinked to the micropatterns per scan cycle or per unit of optical energy delivered, and hence refers to the efficiency of the multiphoton crosslinking of NA. FIG. 8D (VI) shows that the NA crosslinking efficiency is NA concentration-dependent with linear association at different laser powers. Laser power, however, associated with the NA concentration-dependent crosslinking efficiency in a significant, positive and non-linear manner (P=0.008 in non-linear curve fitting and R²ₑₓₚₒₙₑₙₜᵢₐₗ=0.997) (FIG. 8D (VII)). To demonstrate that the NA-biotin binding process is also quantitatively controllable through the multiphoton-based NA micropatterning process through the laser parameters (scan cycle and power), the total fluorescence of the NA-bound Atto 655-Biotin is converted to its concentration by estimating from a standard/calibration curve of the total fluorescence of the NA-bound biotin against a series of known concentrations of Atto 655-Biotin. FIG. 8E (I) demonstrates that, the multiphoton-based NA crosslinking process indirectly determines the amount of Atto 655-Biotin bound in a positive and linear manner although the sensitivity of the NA-biotin binding (slopes of curves in FIG. 8E (I)) associated with the laser power in a non-linear manner (P 0.000 in non-linear curve fitting and R²_{cubic}=0.9187) (FIG. 8E (II)).

Spatially and Quantitatively Controlled Micropatterning of BMP-2 Via Biotin-NA Interactions

The multiphoton microfabrication is shown to create NA micropatterns and the subsequent functional binding to biotin in a spatially and quantitatively controllable manner (FIG. 9A-E). Biotin is a small molecule known to be able to label many delicate soluble factors. Conjugation of the soluble niche factor of interest with biotin molecule, known as biotinylation, with optimal level of conjugation and without compromising its NA-binding ability, is a notable step in the current platform. Using BMP-2 as the example, the growth factor is biotinylated, the level of labeling is optimized, and the functions of both biotin and the BMP-2 are verified through NA binding and Smad signaling, respectively (FIG. 9A (I)). The molar ratio of NHS-PEG₄-Biotin to BMP-2 is selected as the major variant of the biotinylation experiment owing to its determinant role in the level of biotin conjugation, and it is found that the level of labeling, abbreviated as LOL hereafter, is positively associated with the increasing molar ratio of NHS-PEG₄-Biotin to BMP-2 applied in the initial reaction (FIG. 9A (II)), R²_{quadratic}=0.9328), and at most 12 biotins can be tagged on one BMP-2 molecule (i.e., LOL is 12) when the molar ratio of NHS-PEG₄-Biotin to BMP-2 applied is as high as 40:1. Further indirect enzyme-linked immunosorbent assay (ELISA) showed that biotinylated BMP-2, abbreviated as bBMP-2 hereafter, can be successfully immobilized on the commercial NA-coated surface, and moreover, the amount of the NA-immobilized BMP-2 is positively associated with the increasing concentration of bBMP-2 applied, as well as the LOL of bBMP-2 (FIG. 9A(III)); in particular, lower LOL of bBMP-2 (2 and 4) shows a good linear controllability over the amount of the NA-immobilized BMP-2 (R²ₗᵢₙₑₐᵣ=0.998 for LOL of 2 and R²ₗᵢₙₑₐᵣ=0.992 for LOL of 4). The bioactivity assay proves that bBMP-2 remains bioactive to trigger Smad 1/5/8 nuclear translocation in mouse myoblasts (C2C12 cell line) after addition of bBMP-2 with LOL of 4 (data not shown, and FIG. 9B) and other LOL (2 and 12) in the culture media. These data collectively suggest that biotinylated BMP-2 with a lower LOL (i.e., 2 or 4) is preferable for the downstream applications.

The multiphoton microfabrication is shown to create NA micropatterns and the subsequent functional binding to biotin in a spatially and quantitatively controllable manner. Results in FIG. 8A-E demonstrate that the current platform is able to spatially and quantitatively control the NA micropatterns, and hence, it is possible to create BMP-2 micropatterns by attaching biotinylated BMP-2 to the NA micropatterns through biotin-NA interactions (FIG. 9C). Representative immunofluorescence images demonstrate firstly, the spatial controllability of the BMP-2 via biotin-NA interactions as the immuno-positive BMP-2 signal is only confined to the “BMP2”-shaped NA micropatterns but not the surrounding NA-free BSA matrix only area (dark); secondly the high specificity of the biotin-NA binding between the biotinylated BMP-2 and the NA micropatterns as the immune-positive signal from the bBMP-2 micropatterns is much higher than that from the unlabeled BMP-2 (abbreviated as fBMP-2 hereafter) counterpart and the PBS control (data not shown Moreover, the data also showed that the quantity of the micropatterned BMP-2 can be readily controlled by varying the number of scan cycles during NA micropatterning, represented by the gradually decreased immune-positive signal along the string of letters “B”, “M”, “P” and “2” at descending numbers of scan cycle (15, 11, 7 and 3). Furthermore, quantitative measurements of immunofluorescence intensity of BMP-2-bound NA square micro-matrix reveals the controllability of laser factor (i.e., scan cycle during the NA micropatterning step) and reagent factor (i.e., amounts of BMP-2 loaded to the NA micropatterns during the biotin-NA binding step) over the quantity of NA-bound BMP-2 (FIG. 9D-E and data not shown). Specifically, the quantity of the NA-bound bBMP-2 (measured from the immunofluorescence of square micro-matrix is significantly, positively and non-linearly associated with the ascending amounts of BMP-2 loaded to the NA micropatterns fabricated at a particular number of scan cycle (R²_{cubic}>0.96 in all scan cycles), and in parallel, it increases with the ascending scan cycles applied in the NA micropatterns fabrication when the bBMP-2 for NA binding is applied at a particular amount (non-overlapped fitting curves of each scan cycle in FIG. 9D). However, there is no such controllability of either the laser factor or the reagent factor over the quantity of NA-bound fBMP-2 (overlapped fitting curves of each scan cycle in the lower right-hand panel of FIG. 9E), indicating that the increased amount of NA crosslinked to the system results in a non-linear increase of the amount of BMP-2 bound afterwards, and specific NA-biotin binding is required for the controllable micropatterning of BMP-2.

Sustained and higher level of Smad signaling triggered by the micropatterned BMP-2 The canonical BMP signaling pathway is featured by the nuclear translocation of phosphorylated receptor-regulated small mothers against decapentaplegic 1, 5 and 8 (or 9) (R-Smad 1/5/8(9); Smad 8 and Smad 9 are alternative names for the same protein) triggered by the binding event between the BMP ligand and the cell-membrane bound serine/threonine BMP receptors. Thus, the pSmad 1/5/8 nuclear accumulation, measured as the Nucleus/Cytosol ratio (abbreviated as N/C ratio hereafter), is a sensitive marker of the activated signaling event triggered by BMP ligand-receptor binding. The co-localization of the pSmad 1/5/8 with nuclear stain DAPI in mouse myoblasts (C2C12 cell line) cultured on the bBMP-2 (LOL=4) bound NA square micro-matrix for 24 hours (data not shown) revealed the nuclear accumulation of pSmad 1/5/8, demonstrating the undisturbed bioactivity of the bBMP-2 after immobilization to the NA micropatterns. Nuclear accumulation of pSmad 1/5/8 in the fBMP-2 group (data not shown) verifies the positive results while the medium group, serving as a negative control, shows no nuclear accumulation but diffused pSmad 1/5/8 throughout the cells (data not shown).

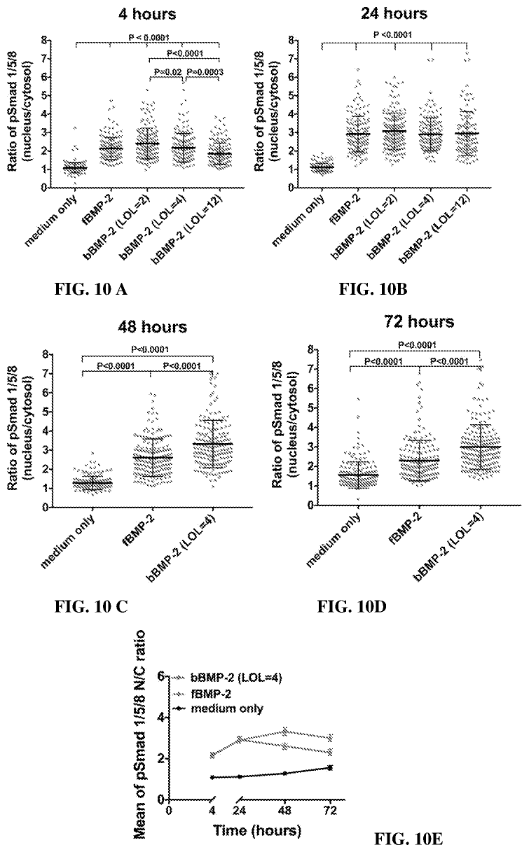

Furthermore, quantitative analysis on N/C ratio unveils that the NA-bound bBMP-2 with different LOL (2, 4 and 12) is equally effective as that of fBMP-2 in triggering pSmad 1/5/8 nuclear accumulation, in both short- (4 hours, FIG. 10A) and long-term (24 hours, FIG. 10B), as compared with negative control (For 4 hours group, One-way ANOVA: F (4, 790)=108; P<0.0001; Bonferroni's post-hoc tests: P=0.0001 for medium only group vs. fBMP-2 and bBMP-2 groups. For 24 hours group, One-way ANOVA: F (4, 680)=113.2; P<0.0001; Bonferroni's post-hoc tests: P=0.0001 for medium only group vs. fBMP-2 and bBMP-2 groups). Moreover, the LOL impacted the N/C ratio in short-term, as shown that the N/C ratio gradually decreases with the increasing LOL of bBMP-2 in 4 hours group (FIG. 10A) and the differences among these groups are significant (One-way ANOVA: F (2, 465)=21.61; P<0.0001; Bonferroni's post-hoc tests: P<0.02 for bBMP-2 (LOL=2) group vs. bBMP-2 (LOL=4) group vs. bBMP-2 (LOL=12) group); however, these differences are no longer significant in 24 hours group (FIG. 10B, One-way ANOVA: F (2, 409)=1.22; P=0.297; Bonferroni's post-hoc tests: P>0.3 for bBMP-2 (LOL=2) group vs. bBMP-2 (LOL=4) group vs. bBMP-2 (LOL=12) group), indicating that the high LOL adversely affects cell's response to the NA-bound bBMP-2 in early time point, corroborating with earlier result (data not shown) that a high LOL is not preferred.

Notably, the NA-bound bBMP-2 results in a more sustained signaling, as shown by a significantly higher N/C ratio at both 48- and 72-hours post-seeding of C2C12 cells, than that induced by the fBMP-2 (For 48 hours group (FIG. 10C, One-way ANOVA: F (2, 488)=184; P<0.0001; Bonferroni's post-hoc tests: P<0.0001 for medium only group vs. bBMP-2 (LOL=4) group vs. fBMP-2 group; For 72 hours group (FIG. 10D), One-way ANOVA: F (2, 506)=90.22; P<0.0001; Bonferroni's post-hoc tests: P<0.0001 for medium only group vs. bBMP-2 (LOL=4) group vs. fBMP-2 group). Two-way ANOVA further demonstrates that there is a statistically significant interaction between the effect of treatment factor (cells treated without BMP-2, with fBMP-2 and with bBMP-2) and time factor (observation duration post-seeding of cells) on the N/C ratio (FIG. 10E, F (6, 1897)=20, P<0.0001). The simple effects analysis shows that the temporal changes of the N/C ratio in bBMP-2 and fBMP-2 groups are different. Specifically, NA-bound bBMP-2 induces an increase in pSmad 1/5/8 nuclear accumulation from 4 to 48 hours, at which the magnitude of N/C ratio reaches a peak, and drops at 72 hours to a similar level as that of the 24 hours' time point (red line, Bonferroni's post-hoc tests: P<0.0001 for 4 hours group vs. 24 hours group vs. 48 hours group; P=0.0023 for 48 hours group vs. 72 hours group; P>0.9999 for 72 hours group vs. 24 hours group); in contrast, the magnitude of N/C ratio in fBMP-2 group peaks at 24 hours and decreases gradually to the similar level as that of the 4 hours' time point until 72 hours (blue line, Bonferroni's post-hoc tests: P<0.01 for 4 hours group vs. 24 hours group vs. 48 hours group; P=0.004 for 48 hours group vs. 72 hours group; P=0.534 for 72 hours group vs. 4 hours group). What is more important is that, NA-bound bBMP-2 and fBMP-2 triggers a similar magnitude of N/C ratio at both time point of 4 and 24 hours (Bonferroni's post-hoc tests: P>0.9999 for bBMP-2 group vs. fBMP-2 group at both 4 and 24 hours) while the N/C ratio in the bBMP-2 group becomes higher after 24 hours (Bonferroni's post-hoc tests: P<0.0001 for bBMP-2 group vs. fBMP-2 group at both 48 and 72 hours). All these data demonstrate that the NA-bound BMP-2 is able to trigger a more sustained Smad signaling and results in a higher magnitude of cell response to the BMP-2, as compared to the soluble BMP-2 supplemented to the culture medium.

Spatially and Quantitatively Controlled Smad Signaling by Micropatterned BMP-2

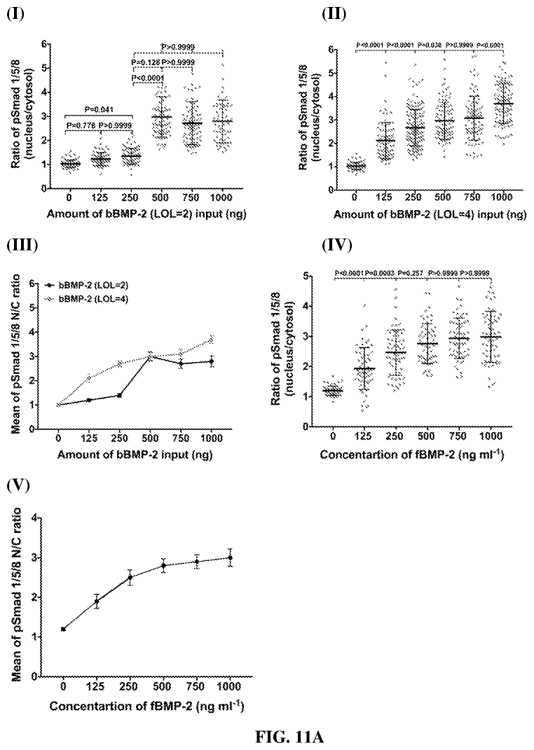

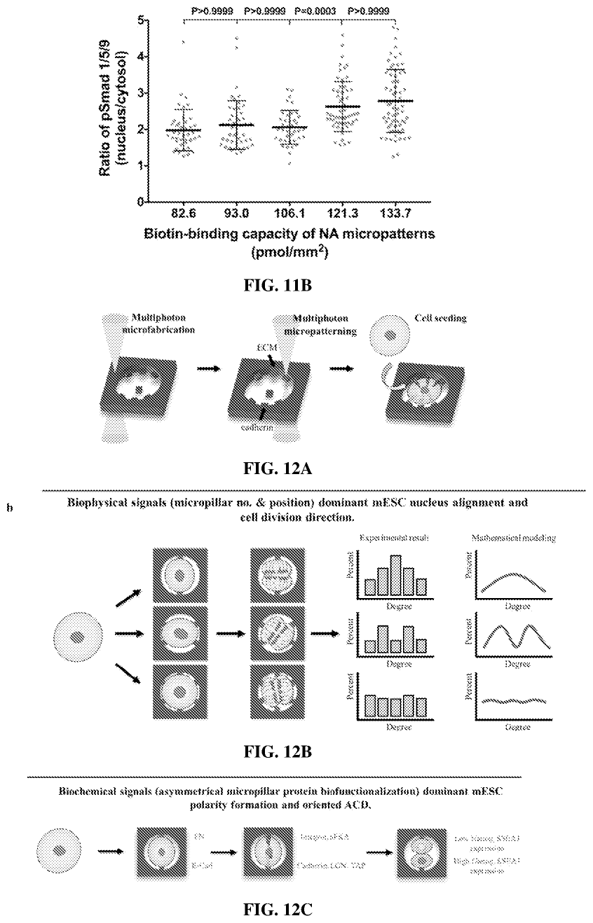

It is further demonstrated the capability of the current platform to spatially control the BMP signaling as cells with nuclear accumulation of pSmad 1/5/8 are exclusively confined on the NA micropatterns (denoted by white dashed squares) but not on the NA free (BSA substrate only) regions (data not shown). Apart from the spatial controllability, the platform is also capable to quantitatively control the N/C ratio either through the ligand factor (BMP-2 concentration) (FIG. 11A) or the laser factor (scan cycle for NA micropatterning) (FIG. 11B). FIG. 11A (I) shows a statistically significant increase in the pSmad 1/5/8 nuclear accumulation (One-way ANOVA: F (5, 456)=149; P<0.0001) as the bBMP-2 (LOL=2) concentration increases to 250 ng (Bonferroni's post hoc tests: P=0.041 for 250 ng group vs. 0 ng group), followed by a rapid increase at 500 ng (Bonferroni's post-hoc tests: P<0.001 for 500 ng group vs. 250 ng group) and then levels off thereafter (Bonferroni's post hoc tests: P>0.1 for 500 ng group vs. 750 ng group vs. 1000 ng group), showing a “switch-like” response (black line in FIG. 11A (III)). At a higher LOL of 4, the ascending dosages of bBMP-2 also result in a statistically significant increase (One-way ANOVA: F (5, 601)=115.4; P<0.0001) in N/C ratio and Bonferroni's post-hoc tests show that all pairs of comparison are statistically significant (P<0.05) except the 500 and 750 ng pair (P>0.9999) (FIG. 11A (II)), indicating that cell responds to the bMP-2 (LOL=4) in a “graded” manner (red line in FIG. 11A (III)), similar to the cell's response to the fBMP-2 gradient (FIG. 11A (IV, V)). Moreover, at each concentration level except 500 ng, the magnitudes of the N/C ratio in the bBMP-2 (LOL=4) group are higher than that in the bBMP-2 (LOL=2) group (simple effects analysis with Bonferroni's post-hoc tests in Two-way ANOVA (F (5, 1057)=23.1; P<0.0001): P<0.005 for 125, 250, 750 and 1000 ng group pairs; P>0.9999 for 500 ng group pairs), indicating that BMP-2 with a mediocre level of biotinylation ensures its efficient binding to the NA substrate. All these data suggest that the bBMP-2 with a LOL of 4 is optimal to activate Smad signaling in a dose-dependent manner.

On the other hand, the bBMP-2 bound to NA micropatterns with different biotin-binding capacities is also shown to have a small but significant impact (One-way ANOVA: F (4, 249)=14.98; P<0.0001) on pSmad 1/5/8 nuclear accumulation, particularly when the biotin-binding capacity is beyond 121.3 pmol/mm² (Bonferroni's post-hoc tests: P>0.9999 for 82.6 vs. 93.0 vs. 106.1 pmol/mm² group; P=0.0003 for 106.1 vs. 121.3 pmol/mm² group; P>0.9999 for 121.3 vs. 133.7 pmol/mm² group) (FIG. 11B), indicating that BMP-2 gradient achieved by varying the biotin-binding capacity of NA is narrow but still effective to trigger a BMP-2 dose-dependent cell response.

Disclosed is a multiphoton-based micropatterning of soluble cell niche factor, using BMP-2 as an example, on protein microstructures via biotin-avidin interactions, enabling the recapitulation of heterogeneous soluble cell niche, in both spatial location and local density-dependent manner, as that present in native tissues. This disclosure also demonstrates the ability of immobilized BMP-2 to triggering a more sustained and a higher level of downstream Smad signaling, comparing to its counterpart supplemented in the culture medium, contributing to the long-term goal of achieving a truly programmable cell niche platform. Existing micropatterning technologies utilizing the biotin-avidin interactions are effective in micropatterning the biomolecules of interest in a controllable manner and ensure the bioactivity of the target molecules after patterning. In this process, neutravidin (NA) can be facilely free-written on the BSA substrates along with the two-photon laser path in a quantitatively controllable manner by simply adjusting the laser power, scan cycle and NA concentration, without the need of pre-modifications on the native proteins (e.g., BSA and NA), toxic substances, and complicated preparation of photomask or PDMS stamp. More importantly, NA is still bio-functional to bind biotin molecules afterwards (FIG. 8B-E and FIG. 9D). It is shown that the bioactivity of BMP-2 cannot be maintained after the multiphoton-induced direct photochemical crosslinking, indicating that, unlike extracellular proteins (ECM) or NA as in the current disclosure, delicate growth factors are susceptible to the photochemical-induced protein-protein crosslinking, possibly due to its mono bio-functional site or unique specific conformational structure disrupted in the course of crosslinking. As a result, resorting to an indirect way without disturbing critical structure of such vulnerable molecules, such as introducing linking system like the biotin-avidin pair as shown here, is necessary.

It is in some embodiments essential to optimize the biotinylation process to ensure the biotin-conjugated molecules are both effective for neutravidin binding and bioactive for downstream application. The results demonstrate that one BMP-2 with four biotins attached (i.e., LOL of bBMP-2 is 4) is not only able to trigger Smad signaling in mouse myoblasts in its NA-unbound form (data not shown and FIG. 9B) but also activate a dose-dependent cell response in its NA-bound form (FIG. 11A (II)). This might be attributable to the mediocre molar excess of NHS-PEG₄-Biotin (5:1) used in the reaction, which compensates the insufficient degree of biotin labeling caused by the possible hydrolysis of N-Hydroxysuccinimide (NHS) ester in aqueous reacting system. On the other hand, the fact that preferential reaction happened between NHS esters and α-amines on N-termini rather than that on lysine side chain owing to the lower pKa (7.6 to 8.0) of N-terminal α-amines in the reacting system with moderate pH (7.2), together with the fact that not much NHS-PEG₄-Biotin is present in the reacting system, reduce the possibility of reaction happened on the lysine residues in cysteine-knot domain, a crucial structure for BMP receptor binding, therefore secures the bioactivity of the resultants.

The temporal change of Smad signaling can be a useful readout to describe the cellular response to BMP ligand presented in different forms over time. A body of studies reported that solid-phase BMP-2 (tethered on ECM or other material surface) tends to trigger a more sustained phosphorylation of R-Smads compared to its soluble counterpart (Table 2).

TABLE 2
In vitro Model of ECM-tethered BMP-2 Signaling

| | | | Level of Smad | |
|---|---|---|---|---|
| In vitro model | | | signaling between | |
| mimicking ECM- | Observation | Temporal change of | tethered and non- | |
| tethered BMP-2 | period | the Smad signaling | tethered BMP-2 | Detection method |
| rhBMP-2 covalently | 15 min-48 h | pSmad 1/5/8 | Not compared | Western blot (WB) |
| conjugated to type I | | expression in: | | |
| atelocollagen [50] | | (1) Tethered BMP-2 | | |
| | | group: observed at 48 h | | |
| | | (2) Non-tethered BMP-2 | | |
| | | group: not detectable | | |
| | | at 48 h | | |
| rhBMP-2 | 30-60 min | pSmad 1/5 expressed | Comparable for WB | WB, |
| micropatterned on | for WB; | in both tethered and | data, different | Immunofluorescence |
| the microcontact- | 10-90 min | non-tethered BMP-2 | patterns for IF data | staining (IF) |
| printed biotin- | for IF | after 30 min (WB); | | |
| fibronectin surface | | Nuclear accumulation | | |
| via biotin- | | of pSmad 1/5 (IF) in: | | |
| neutravidin-biotin | | (1) Tethered BMP-2 | | |
| bridge [30] | | group: increased at | | |
| | | 10 min and dropped | | |
| | | thereafter and | | |
| | | increased again at | | |
| | | 45 min | | |
| | | (2) Non-tethered BMP-2 | | |
| | | group: peaked at 30 min | | |
| | | and dropped thereafter | | |
| rhBMP-2 covalently | 30-180 min | pSmad 1/5/8 | Tethered BMP-2 > | WB |
| conjugated to the | | expression in: | non-tethered BMP-2 | |
| gold nanoparticles | | (1) Tethered BMP-2 | only in the presence | |
| decorated substrate | | group: delayed to 90 | of lowest amount of | |
| [42] | | min and sustained to | ligand | |
| | | 180 min | | |
| | | (2) Non-tethered BMP-2 | | |
| | | group: increased after | | |
| | | 30 min and decreased | | |
| | | between 90 and 180 min | | |
| rhBMP-2 bound to the | 30-180 min | pSmad 1/5 | Tethered BMP-2 > | WB |
| heparan sulfate (HS) | | expression in: | non-tethered BMP-2 | |
| or the gold surface | | (1) Tethered BMP-2 | after 180 min; | |
| via biotin- | | group: peak delayed to | HS-tethered BMP-2 > | |
| neutravidin-biotin | | 90 min and sustained to | streptavidin- | |
| bridge [51] | | 180 min | tethered BMP-2 at | |
| | | (2) Non-tethered BMP-2 | all time-points | |
| | | group: decreased in a | | |
| | | time-dependent manner | | |
| | | within 180 min | | |
| rhBMP-2 micropatterned | 4-72 h | Nuclear accumulation of | Comparable at 4 and | IF |
| on the neutravidin | | pSmad 1/5/8 (IF) in: | 24 h; tethered BMP- | |
| micropatterns | | (1) Tethered BMP-2 | 2 > non-tethered | |
| fabricated by MMM via | | group: peaked at 48 h | BMP-2 after 24 h; | |
| neutravidin-biotin | | and declined gradually | tethered BMP-2 [symbol] > | |
| binding (the present | | at 72 h | non-tethered BMP-2 [symbol] | |
| study) | | (2) Non-tethered BMP-2 | | |
| | | group: peaked at 24 h | | |
| | | and dropped sharply at | | |
| | | 72 h | | |

[symbol]

indicates data missing or illegible when filed

Here, the results not only support the notion that the immobilized BMP-2 outperformed its soluble counterpart, but also achieve a sustained Smad signaling up to 72 hours, much longer than the time window reported in all studies in Table 2. Additionally, it is revealed that the level of the nuclear accumulated pSmads triggered by NA-bound bBMP-2 is approximately 13.7% higher than that by fBMP-2, which has not been reported before. The sustained high pSmad 1/5/8 N/C ratio and the subsequent continuous accumulation of pSmad 1/5/8 in the nucleus, may suggest continuous activation of BMP-receptor on the cell surface. Therefore, it is speculated that the NA-bound BMP-2 continuously triggers BMP-receptor (BMPRI and BMPRII) complex formation on the plasma membrane, which might be sufficient and even more efficient to transduce BMP signal into the cell, as compared to the traditional BMP signal propagation via a clathrin-mediated endocytosis of BMP-BMPRI/BMPRII complex triggered by soluble BMP-2, where BMP-2 ligands are continuously consumed and BMP-2 receptor recycling to the cell surface for next round of signaling transduction are time consuming. BMPs are known to have short half-life and rapid clearance rate, as a result, the effective amount of soluble BMP-2 to trigger cell response in the present disclosure might decrease over time; while the NA-bound bBMP-2 is localized to the micropatterns, and do not release to the culture medium over 7 days as measured by sandwich ELISA, providing a stable amount of effective BMP-2 to activate signaling event. Additionally, BMP signaling is negatively regulated by antagonists (e.g., Noggin) at extracellular level, hence it is possible that these antagonists recognize BMP-2 presented in different forms (i.e., soluble form and NA-bound form) in a different manner, resulting in a distinct pattern of signaling pathway subsequently. Altogether, the current engineered soluble cell niche is able to reconstitute heterogeneous soluble niche factor with spatial and quantitative control as that in native cell niche, including, but not limited to, BMP, Hedgehog and Wnt, which are essential signaling molecules for organogenesis as well as tumorigenesis. Thus, the present disclosure represents a powerful cell niche engineering platform to reconstitute a biomimetic soluble cell niche to sustain or even potentiate the effectiveness of bioactive molecules in vitro, facilitating the elucidation of basic biological questions during physiological and pathological processes.

Establishing an in vitro model able to spatially control the concentration gradient of soluble signals for multiple applications including cell niche studies remains an outstanding challenge. Multiple techniques, such as surface modification with or without microcontact printing, or layer-by-layer (LbL) integrated with microfluidics, have been utilized to generate continuous or discrete BMP-2 gradients in recent years. However, simultaneous spatial and quantitative control of BMP-2 by a single platform is yet to be achieved. In the current disclosure, the soluble niche factor (BMP-2 for instance here) can be arbitrarily patterned by the MMM technology on BSA substrate via NA-biotin binding pair at micron-scale. In the meantime, the concentration gradient of BMP-2 can be readily generated by controlling the amount of bBMP-2 applied to the NA micropatterns (FIG. 11A(II)), or by changing the biotin-binding capacity of the NA micropatterns via changing the number of scan cycle during NA micropatterning (FIG. 11B). Although the BMP-2 concentration gradient engineered by the latter strategy triggers a narrower range of differential nuclear accumulation of pSmads in C2C12 cells than expected, possibly due to a narrower actual range of NA-bound BMP-2 resulted from its much larger molecular size than Atto 655-Biotin for estimation, this MMM platform allows to mimic the heterogeneity of soluble cell niche, providing a valuable in vitro tool to recapitulate native biological activities such as morphogen concentration-gradient induced tissue pattern formation during early developmental process. Another advantage of MMM platform is that the cellular activity can be considered as the response exclusive to the NA-bound soluble factors, therefore, it is easy to integrate the soluble niche with other niche factors, such as mechanical niche and matrix niche established before, to engineer a complex cell niche model.

The current disclosure reports a robust multiphoton microprinting-based platform to micropattern soluble niche factors, as exemplified by BMP-2, by three steps: (1) microfabricate a BSA substrate followed by micropatterning NA on its top; (2) chemically conjugate biotin to BMP-2; (3) generate BMP-2 micropattern in register to NA micropattern via NA-biotin binding pair. This technology represents a microfabrication and micropatterning-integrated all-in-one platform without sophisticated surface modification and photomask preparation, and more importantly, simultaneously accomplishes the spatial and quantitative control of the patterned soluble factor. The micropatterned BMP-2 remains bioactive in eliciting Smad signaling and provokes a more sustained and a higher level of Smad signaling than the soluble BMP-2 counterpart. These results pave the way for creating an artificial soluble niche with spatially, continuously and quantitatively presented growth factor or morphogen, to facilitate in vitro studies on the associated signaling events, morphogen gradient-dependent pattern formation, and to integrate with other niche components to achieve the long-term goal of establishing a truly programmable cell niche engineering platform.

3. Controlling the Orientation of the Asymmetric Cell Division (ACD) of Mouse Embryonic Stem Cell (mESCs) in a Micro-Printed 3D Single Cell Micro-Niche

Multiple Cell Niche Signals Affect Early Development

During early development, multiple niche factors, including diffusive signals such as soluble morphogens and growth factors, local biochemical signals such as extracellular matrix and cell adhesion molecules and local biophysical signals such as geometry and tensile force, orchestrate in a well-organized manner to direct tissue patterning and morphogenesis (Sozen et al., 2020; Vining and Mooney, 2017).

Local Niche Factors Affect Cell Fate Determination

The roles of local niche signals in affecting cell fate determination during early development have been drawing increasing attention of researchers (Landge et al., 2020). Geometrical constraints by cell-cell mechanical coupling affected cell shape and tissue geometry, and promoted epithelial morphogenesis (Xiong et al., 2014). Small difference in cellular tension between two embryonic stem cells influenced their positioning and fate specification, and governed self-organization of blastocyst (Maître et al., 2016). Extracellular matrix signals such as laminin and fibronectin regulated the polarization of embryonic cells and subsequent morphogenesis (Bedzhov and Zernicka-Goetz, 2014), and promoted the emergence of mesodermal cells during development (Cheng et al., 2013). Cell-cell interaction molecules such as cadherin enabled precise tissue patterning through formation of sharply bordered neural tube domains (Xiong et al., 2013) and self-organized cell sorting (Toda et al., 2018).

Cell Division Polarity Represents Critical Mediator for Cell Fate Specification

Cell division polarity represents an important mediator of local niche signals-induced cell fate changes. Local mechanical niche signal such as stretching force can deform the nucleus, resulting in chromatin organization and transcription activation (Miroshnikova and Wickström, 2021). For examples, deformation of nucleus from an isotropic circular geometry to an anisotropic elongated geometry via micropatterning promoted nuclear elongation and reduced differentiation of keratinocytes (Connelly et al., 2010) and epidermal progenitor cells (Miroshnikova et al., 2018). During cell division, the contractile force generated through mechanical interaction between cell and its microenvironment pulling apart the mitotic spindles to split the genetic materials of the dividing cell into two nucleus, which forms the cell division direction, as determined by the orientation of two daughter cell nucleus position (Lesman et al., 2014; Lough et al., 2019). Proper cell division direction assures effective tissue morphogenesis and homeostasis. For examples, perpendicular cell division direction facilitated skin stratification and differentiation during embryonic epidermal development while knocking out the epidermal transcription factor disrupted perpendicular cell division direction and abolished multi-layered stratification and differentiation of keratinocytes (Lechler and Fuchs, 2005). A special type of cell division, asymmetric cell division (ACD), dictates the unique capability of stem cells to undergo proliferation and differentiation at the same time, resulting in differential fates of the two daughter cells, one stem cell and one differentiate or committed cell. Proper orientation of the ACD governs correct tissue morphogenesis, patterning and homeostasis. On the contrary, ACD polarity/orientation defects generated through deletion of critical regulators such as Par3 are associated with epithelial tumor formation (McCaffrey et al., 2012) while mutations of these polarity regulators also resulted in cancer-associated phenotype in mouse models (Gomez-Lopez et al., 2014; Neumüller and Knoblich, 2009).

Cell Niche Engineering Technologies

Microfabrication and micropatterning technologies have been employed to develop in vitro models to study how cell niche signals affect cellular activities including cell division polarity (Lutolf et al., 2009). For example, 2D micropatterned glass surfaces with matrix coating via microcontact printing or 3D nonadherent PDMS chamber were able to control the division direction of HeLa cells or sea urchin eggs (Minc et al., 2011; Thery et al., 2007). However, conventional microcontact printing or mold-based fabrication techniques could only provide global or macroscale signals to cell populations and have difficulties to incorporate anisotropic/asymmetric local niche signals at the immediate microenvironment of cells. Emerging multiphoton-based microfabrication technologies have great potential in engineering heterogenous 3D microstructures (Harper et al., 2012) for biomedical applications such as fabricating heterogeneous protein structures to guide neurite formation from dorsal root ganglion (Kaehr et al., 2004). Our lab has previously developed a multiphoton microfabrication and micropatterning (MMM) technology to engineer anisotropic/asymmetric protein cell niches through fabricating a wide spectrum of user-defined complex protein microstructures, with spatial and quantitative control over their geometry and morphology (Chan et al., 2014; Ma et al., 2017), mechanical properties such as modulus and stiffness (Tong et al., 2017; Tong et al., 2016), and biochemical properties such as extracellular matrix (ECM) (Huang et al., 2018) and morphogenic proteins (Wang et al., 2021).

Methods

Fabrication of Protein-Based 3D Single Cell Micro-Niche

The fabrication process was conducted using a confocal laser scanning microscope (Carl-Zeiss 710) with a mode-locked Ti:sapphire femtosecond near infrared laser (Coherent, Santa Clara, California, USA), a 40× oil immersion objective lens (numerical aperture=1.3) and laser wavelength of 800 nm. BSA (Sigma-Aldrich) and RB (Sigma-Aldrich) were freshly prepared and mixed to concentration at 300 mg/mL BSA with 0.1% w/v RB before experiment. The 3D micro-niche structures were fabricated inside a glass-bottomed 35 mm confocal dish (P35G-1.5-10-C; Ashland, MA, USA). The 3D micro-niche structures were fabricated in a stepwise process, following the sequence of micro-niche wall, the bottom micropillar, the wall micropillar, achieved by utilizing the region of interest (ROI) function of ZEN 2009 software. For most of the micro-niche used in this experiment, the size of micro-niche was designed as follow. The micro-niche outer wall length was 37 μm and the height was 20 μm, the inner aperture diameter was 20 μm. The distance between corresponding pillars was 12 μm, and the z-axis distribution was from 10 to 13 μm from glass surface. The length of bottom pillar was 4 μm and the height was 3 μm. For the tilted pillar micro-niche, the micro-niche outer wall length was 37 μm, the inner aperture diameter was 20 μm, the wall height was 20 μm, and the z-axis distribution of two corresponding pillars was from 3 to 6 μm and from 10 to 13 μm. For the micro-niche used in the 6, 4, and 2 FN-functionalized micropillar micro-niche part, the outer wall length was 28 μm and the height was 20 μm, the inner aperture diameter was 15 μm. The distance between corresponding pillars was 10.2 μm, and the z-axis distribution was from 10 to 13 μm from glass surface. The length of bottom pillar was 3 μm and the height was 3 μm. The laser power was calibrated every time before experiment and set as 175 mW. After fabrication, the 3D micro-niche was washed 3 times in PBS to remove the unreacted BSA and RB. 3D micro-niche was kept in 4° C. until use.

Functionalization of Wall Micropillars

FN (F-2006; Sigma-Aldrich) was mixed with RB to reach the final concentration at 0.9 mg/mL with 0.1% w/v RB. Protein A/G (21186; Thermo Scientific) was mixed with RB to reach the final concentration at 0.56, 1.13, 2.25 to 4.5 mg/mL with 0.1% w/v RB. ROI was specifically designed and aligned to the target wall micropillar locations. Laser power used for crosslinking was 48 mW and the scan cycle were set to 5. After crosslinking and coating, the 3D micro-niche was washed 3 times in PBS to remove the unreacted FN, Protein A/G and RB. For Protein A/G, an extra step of E-Cad-Fc (E2153; Sigma) incubation was applied for 1 hour before cell seeding and cell culture. After incubation, the 3D micro-niche was washed for another 3 times with PBS to remove unbound E-Cad-Fc. For the quantification of Protein A/G crosslinking density, Alexa Fluor 633 donkey anti-goat antibody (A-21082; Invitrogen) was allowed to bind to crosslinked Protein A/G overnight in 4 degree refrigerator. For the quantification of E-Cad-Fc binding density, a Dylight 633 Fast Conjugation kit (ab201802; Abcam) was used for fluorescence molecule labeling of E-Cad-Fc. The labeling process was conducted following the manufacture's protocol. After labeling, the fluorescence molecule labeled E-Cad-Fc was diluted to 25, 50, 100 and 200 μm/mL for binding experiments. Crosslinking of BSA to the target BSA microstructure surface was used as control. The 3D micro-niche after functionalization was kept in PBS with 5% penicillin and streptomycin (15070063; Gibco) in 4° C. refrigerator until use.

Cell Culture

Mouse embryonic stem cells (L4) were obtained from the Transgenic Core Facility, Department of Biochemistry, The University of Hong Kong. L4 mESCs were used in this experiment for cell alignment, cell division direction and ACD studies in 3D micro-niche. mESCs were cultured and maintained using N2B27 full medium on gelatin-coated cell culture plate. N2B27 basal medium consists of one volume of DMEM/F12 medium (11320033; Gibco) combined with one volume of Neurobasal medium (21103049; Gibco) supplemented with 0.5% N2 Supplement (17502048; Gibco), 1% B27 Supplement (17504001; Gibco), 7.5% BSA solution (15260037; Gibco), 1% penicillin and streptomycin (15070063; Gibco). N2B27 full medium consists of 96.6% N2B27 basal medium, 0.01% PD325901 (04-0006-10; Stemgent), 0.03% CHIR99021 (04-0004-02; Stemgent), 1% Glutamine (35050061; Gibco), 0.2% β-mercaptoethanol (21985023; Gibco), 0.1% Leukemia inhibitor factor (ESG1106; Sigma-Aldrich), 2% inactivated ES-FBS (10439016; Gibco). Cell density for 3D micro-niche based experiments was 4e5 cells/mL. For the cell nucleus alignment, cell area and cell shape measurement, and cell polarity experiments, cells were allowed for residing and binding to the wall micropillars for about 6-8 hours until they formed strong cell attachment to avoid detachment during sample washing and fixation. For the cell division and ACD experiments, cells were cultured for longer time, which was 14 hours in 3D micro-niche for generating two daughter cells. MCF-7 (HTB-22) cells were purchased from ATCC and maintained in complete growth medium composed of DMEM/F12 (11320033; Gibco) supplemented with 10% fetal bovine serum and 1% penicillin and streptomycin (15070063; Gibco). MCF-7 cells were used for the bioactivity verification of E-Cad-Fc according to the manufacturer's instruction. MCF-7 cell density used for cell seeding was 4e5 cells/mL and the cell culture time was 4 hours until they formed strong attachment.

IF Staining

Cells were fixed using 4% paraformaldehyde for 20 mins. The sample was blocked with 3% BSA solution, then incubated with primary antibody at 4° C. overnight. On the next day, the sample was washed and incubated with 3% BSA containing the secondary antibody at room temperature for 1 h, after which they were washed and immersed in mounting medium with DAPI until imaging. The antibodies used in this experiment: aPKC antibody (sc-17781; Santa Cruz Biotechnology; 1:200), Pan-Cadherin antibody (717100; Invitrogen; 1:200), β catenin antibody (ab16051; Abcam; 1:200), α/β-tubulin antibody (CST-2148; Cell Signaling Technology; 1:400), Alexa Fluor 488 Phalloidin (A12379; Invitrogen; 1:40), activated integrin β1 antibody (553715; BD Pharmingen; 1:100), Phospho-FAK (Tyr397) antibody (44-625G; Invitrogen; 1:200), LGN antibody (ab84571; Abcam; 1:200), YAP1 (63.7) antibody (sc-101199; Santa Cruz Biotechnology; 1:200), Nanog antibody (ab80892; Abcam; 1:200), SSEA1 antibody (ab16285; Abcam; 1:200), Vimentin antibody (ab8978; Abcam; 1:200).

Imaging, Cell Area, Cell Shape, Cell Alignment, Cell Division Direction and ACD Analysis

Confocal fluorescence images were taken using Carl-Zeiss 710 and Nikon A1R MP. Cell alignment was analyzed using ImageJ-Fit ellipse function, which automatically found the best fit ellipse for cell nucleus and reported the long axis of the best fitted ellipse. Cell division direction was analyzed using ImageJ-Center of mass function, which automatically found the center of mass of nucleus of two daughter cells and reported the x-y coordinates. The direction of the line that connected the two center of mass points were defined as the cell division direction. Cell area and cell shape were quantified using analyze particles function in ImageJ. Cell shape quantification included roundness, aspect ratio, circularity, and solidity.

Roundness=(4*area)/(π* [symbol] major axis [symbol] \hat{ }2)

Aspect ratio=(major axis)/(minor axis)

Circularity=(4π*area)/ [symbol] perimeter [symbol] \hat{ }2

Solidity=area/(convex area)

ACD was quantified using ImageJ-Measure function. In details, cell nucleuses were transformed to ROI and applied to the Nanog, SSEA1 channels to quantify the fluorescence intensity inside the nucleuses of two daughter cells. All experiments in this study were repeated at least 3 times. FIR was calculated using the following equation.

FIR=(Fluorescence intensity in the bottom cell)/(Fluorescence intensity in the top cell)

Statistical Analyses

All statistical analyses were executed using Origin Pro (2021) (Origin Lab Corporation, Northampton, MA, USA). For image analysis of crosslinked ECM proteins to the BSA micromatrix, Gaussian fitting of the fluorescent intensity distribution data was performed using Origin. P value less than 0.05 was considered statistically significant unless otherwise specified and the exact value was specified in each figure. The sizes of sample were specified in each figure. The quantitative data presented in the figures were shown as mean±s.d unless otherwise specified.

Results

Multiphoton Microfabrication of Single Cell 3D Micro-Niche with Spatially Controlled Asymmetric Biofunctionalization

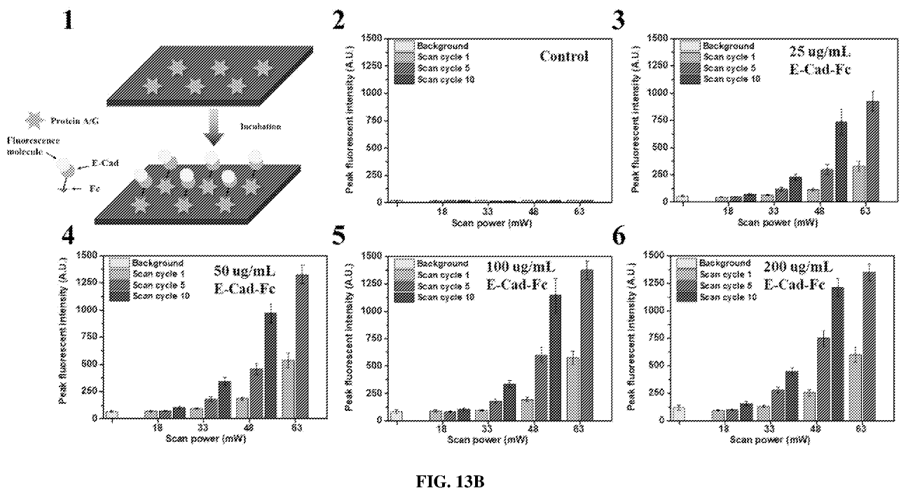

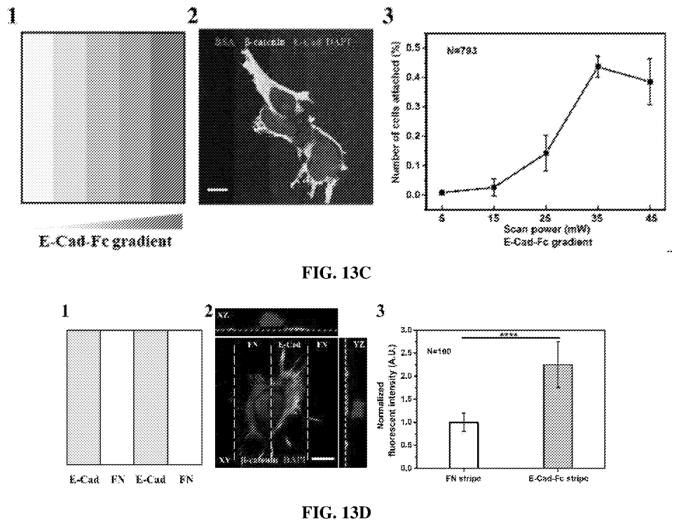

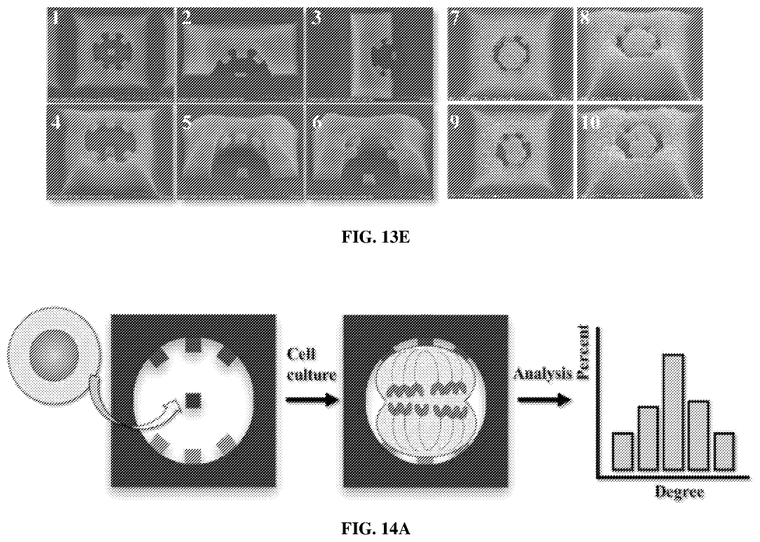

This study aimed to biofunctionalize BSA microstructures with asymmetric local niche signals, specifically using an extracellular matrix protein FN and a cell-cell interaction molecule E-Cad to mimic cell-matrix interaction and cell-cell interaction, respectively. The ECM protein FN was immobilized directly on the surface of the BSA micro-niche as previously reported (Huang et al. 2018). The cell-cell interaction molecule E-Cad is a well-known transmembrane protein with its extracellular domains acting as the homogenous binding sites to the E-Cad expressed by their neighbors. Direct photochemical crosslinking of E-Cad with random molecule orientation might compromise its bioactivity in cell-cell interaction as the functional binding domain might be denatured or not properly oriented, and an indirect immobilization approach was used to photochemical crosslink an adaptor protein namely protein A/G to the surfaces of the BSA substrates before allowing the target protein E-Cad to bind to the adaptor through a unique mechanism (Drees et al., 2005). Specifically, the target protein E-Cad used in the current study is a recombinant protein namely E-Cad-fragment crystallizable (Fc) protein, which is a commercially available fusion protein between mouse E-Cad and a Fc domain of human IgG1. The Fc domain of the E-Cad-Fc protein was able to bind to the adaptor protein A/G, which is known to bind to all human IgG, via their specific and high affinity molecule interactions previously reported (Kovacs et al., 2002). To further improve the adaptor protein immobilization step, protein A/G was photochemically crosslinked to the surface of the underlying BSA microstructures in the micro-niche under different process parameters including laser power, scan cycle and reagent concentration (FIG. 13A). The local density of the immobilized protein A/G was measured as the averaged peak fluorescence intensity signal of an Alexa Fluor 633 Donkey anti-Goat secondary antibody with donkey Fc domain, which binds the protein A/G (FIG. 13A, 1). The clustered bar charts showed the positive dose-dependence of the local density of the immobilized protein A/G against the laser power, the laser scan cycle, and the concentration of the protein A/G (FIG. 13A, 2-6). To assure maximal binding between the adaptor protein AG and the target protein E-Cad-Fc in the subsequent fabrication procedure, an optimal protein A/G concentration of 4.5 mg/ml was selected.

After demonstrating the ability to quantitatively control the local density of protein A/G to the underlying BSA microstructures of the micro-niche, E-Cad-Fc was allowed to bind to the immobilized protein A/G. The E-Cad-Fc, was tagged with a Dylight 633 fluorescence molecule before binding to facilitate direct visualization and quantification (FIG. 13B, 1). The clustered bar charts showed the dose-dependence of the fluorescence intensity of the bound E-Cad-Fc on the laser parameters (scan power and scan cycles) and the concentration of the fluorescence tagged E-Cad-Fc (FIG. 13B, 2-6). To assure maximal binding between the target protein and the adaptor protein, the optimal fluorescence-tagged E-Cad-Fc concentration of 200 μg/ml was selected. Collectively, a laser power at 48 mW, a laser scan cycle of 5, a protein A/G concentration of 4.5 mg/ml and a fluorescence tagged E-Cad-Fc concentration of 200 μg/ml were identified as the optimal specification for the indirect immobilization of the E-Cad-Fc on the micro-niche.

After successful immobilization of E-Cad-Fc to the micro-niche via the adaptor protein A/G, it is important to verify the bioactivity of the immobilized E-Cad-Fc. To assess the bioactivity of the immobilized E-Cad-Fc, MCF-7 cell, which was known to express E-Cad and interact with the extracellular domain of the E-Cad expressed by its neighbor cells (Li et al., 1999), was seeded onto a flat BSA micromatrix surface with E-Cad-Fc density gradient (FIG. 13C, 1). Immunofluorescence staining showed MCF-7 cell attached to the E-Cad-Fc immobilized BSA micromatrix surface (red) expressing E-cad (magenta) and β-catenin (green), and DAPI (blue) was used as nucleus counter stain (FIG. 13C, 2). The number of MCF-7 cells adhered to the E-Cad-Fc immobilized surface was increasing as the local density of the E-Cad-Fc increased and finally saturated (FIG. 13C, 3). Apart from E-Cad-FC density mediated MCF-7 attachment, the bioactivity of the immobilized E-Cad-Fc was also verified by the expression of the downstream marker of E-Cad signaling pathway, β-catenin, which is known to directly bind to the cytoplasmatic tail of bioactive E-Cad to form the cadherin complex (Yap et al., 1997). Specifically, MCF-7 cells were seeded on a micromatrix surface with alternative stripes of FN and E-Cad-Fc (FIG. 13D, 1). First, highly expressed β-catenin (green) was observed in MCF-7 cells adhered on the E-Cad-Fc stripes compared to the FN stripes in the XY image (FIG. 13D, 2). Second, enrichment of β-catenin was only found close to the basal plasma membrane in the XZ and YZ section images (FIG. 13D, 2). This suggests that local enrichment of β-catenin, which is downstream to the E-Cad signaling, was induced upon binding of the MCF-7 cells to the immobilized bioactive E-Cad-Fc. Third, fluorescence intensity of β-catenin of the MCF-7 cells on alternative FN and E-Cad-Fc stripes was further analyzed using ImageJ. A significantly higher expression level of β-catenin (p<0.0001) was found on the E-Cad-Fc stripes than that on the FN stripes (FIG. 13D, 3). These results confirmed that the indirectly immobilized E-Cad-Fc on the micro-niches was bioactive.

By integrating the capability of the MMM platform to microfabricate 3D protein microstructures and micropatterns with spatial control, these studies successful demonstrated of the ability to biofunctionalize the underlying protein structures with ECM and cell-cell interaction molecules with retained bioactivity. Arrays of 3D single cell micro-niche were carefully designed, fabricated and optimized to allow single mESCs to reside, with asymmetric local niche signals, FN and E-Cad functionalizing the interior wall micropillars of the micro-niche was fabricated (data not shown). A stepwise multiphoton-based microfabrication process was used to micro-print the wall, the bottom micropillars and the lateral wall micropillars, before using the same multiphoton-based micropatterning process to bio-functionalize the surfaces of the micropillars with a matrix niche protein (FN, magenta) or a cell niche protein (E-Cad) (data not shown). SEM images were used to demonstrate the 3D microstructure, with top view of a complete (FIG. 13E,1) and two halves (FIG. 13E, 2, 13E, 3) of 3D micro-niches, together with side view of a complete (FIG. 13E,4) and two halves (FIG. 13E, 5, 13E, 6) of 3D micro-niches. High magnification SEM images of micropillars were also demonstrated (FIG. 12D). mESC-seeded micro-niches (light brown) with a single cell from top view (FIG. 13E, 7), and side view (FIG. 13E, 8) or two cells after cell division from top view (FIG. 13E, 9), and side view (FIG. 13E,10) were also demonstrated. The dimensions of the 3D micro-niche structures, both the microwall and the micropillars, were retained in phosphate buffered saline (PBS) for at least two months (FIG. 12E), suggesting their excellent stability. Moreover, the time for fabrication of a single micro-niche was as short as 10-15 seconds while the ROI-based fabrication also enabled simultaneous fabrication of multiple micro-niches, suggesting the high throughput and efficient nature of the MMM platform.

Mechanical but not Biochemical Cues Dominantly Affect Nuclear Deformation and Division Direction of mESC in 3D Biofunctionalized Micro-Niche

Local niche signals are important mediators of proper cell division direction, cell fate changes and assure correct tissue morphogenesis, homeostasis and organ function. The ability to manipulate or control cell division direction is critical in cell niche engineering for tissue engineering and regenerative medicine. Specifically, cell division direction is believed to directly associate with the cytoskeleton force-induced cell nucleus shape and state change (Kirby and Lammerding, 2018) because cell nucleus closely interacts with cytoskeleton components, including actin, intermediate filaments, and microtubules (Chang et al., 2015). Therefore, nucleus deformation could be a good indicator for tensile stress force signal, transduced from the external microenvironment to the intracellular cytoskeleton machinery.

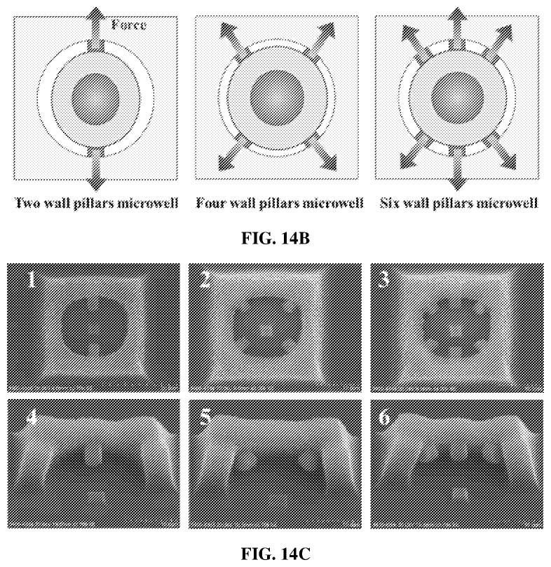

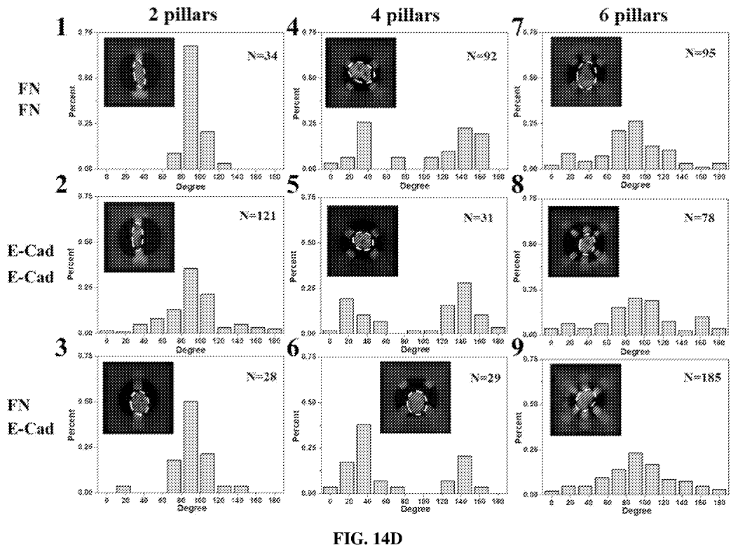

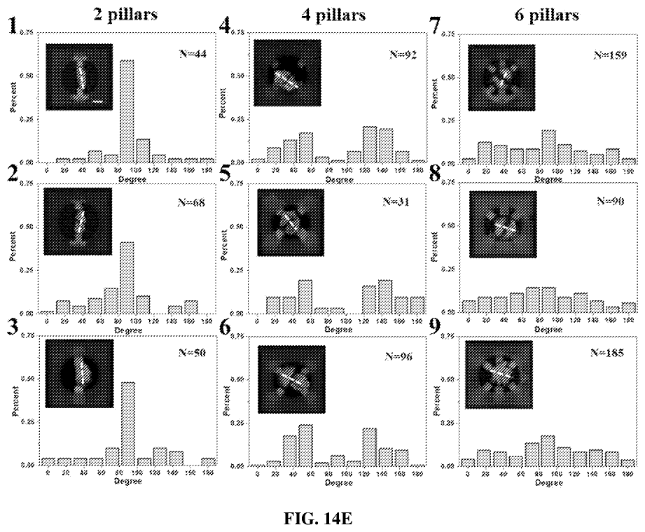

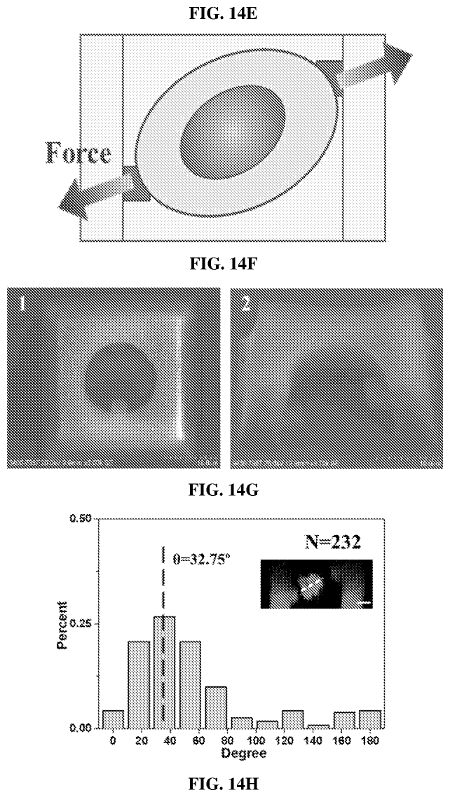

Here, the hypothesis was that, by controlling the direction of the cell attachment and hence the force generated within the mESC through manipulating the design of the micro-niche features, the nucleus deformation alignment and the cell division direction can be precisely controlled. Using the MMM technology, 3D micro-niches with two, four or six interior wall micropillars, biofunctionalized with ECM and cell-cell interaction signals symmetrically or asymmetrically were fabricated, before seeding a single mESC into the micro-niche for subsequent characterization of its nucleus deformation and cell division direction (FIG. 14A). In these 3D micro-niches, two micropillars micro-niche provided the unidirectional cell binding sites and four micropillars micro-niche provided four cell binding sites along two directions, while the six micropillars micro-niche provided two vertically oriented cell binding sites comparing with four micropillars micro-niche, as illustrated by schematic figure (FIG. 14B). Successful fabrication of micro-niche with different pillar numbers including 2, 4, 6, and the relative z axis distribution of micropillars on the wall and at the bottom were shown through SEM images, from both top view (FIG. 14C, 1-3) in full structures and side view (FIG. 14C, 4-6) in half structures. After seeding mESC into the micro-niche, mESCs were stained with cytoskeleton proteins including α/β tubulin (green), vimentin (magenta), cell nuclear using DAPI (blue) and the BSA micro-niches was red. These results showed that during mitosis in mESC, α/β tubulin formed astral microtubules and connecting to chromosomes, which indicated its important role in the cytoplasmic force generation during cell division, while before and after mitosis, it was dispersedly distributed in the cytoplasm (data not shown).

To further characterize nucleus deformation alignment, ImageJ was used to define the long axis of the deformed nucleus upon cell binding to the biofunctionalized micropillars in the micro-niche after 6 hours incubation, by creating the best-fitted ellipse surface, shown as the white dotted circle (FIG. 14D). The horizontal direction, which was vertical to the 2 micropillars direction was defined as 0°. Thus, the direction of the long axis of the nucleus relative to the horizontal direction was defined as the nucleus deformation alignment. The cell division direction was determined as the direction along the center of mass of the nucleus of the two daughter cells immediately after cell division after 14 hours incubation, shown as the white dotted line (FIG. 14E). The hypothesis was that the nucleus deformation and division direction is governed by the directional tensile force generated inside the cells upon attachment to the functionalized micropillars inside the micro-niche. Without biofunctionalization of the micropillars, no cell could attach. In the 2 micropillars micro-niche group, both the nucleus deformation alignment (FIG. 14D, 1-3) and the cell division direction (FIG. 14E, 1-3) were along the same direction of the tensile force generated through binding of the two biofunctionalized anchoring micropillars at 90°. In the 4 micropillars micro-niche group, two major peaks in the alignment in both the nucleus deformation (FIG. 14D, 4-6) and the cell division direction (FIG. 14E, 4-6) were identified while these two peaks aligned well with the diagonal directions of the four micropillars of in the 3D micro-niche, at around 50° and 130°, respectively. In the 6 micropillars micro-niche group, both the nucleus deformation alignment (FIG. 13E, 7-9) and the cell division direction (FIG. 14E, 7-9) showed a rather random distribution but still with a peak at about 90°. The only difference between 4 micropillars micro-niche and 6 micropillars micro-niche is the two additional micropillars at 90°, which indicated the ultra-sensitive nature of cell tensile force generation upon binding to micropillars in our system as small difference in the micropillars design resulting in totally different distribution of cell nucleus deformation alignment and cell division direction. More interestingly, the cell nucleus deformation alignment and cell division direction are independent of either the type or the symmetry of the biochemical niche signals as the nucleus deformation alignment and the cell division polarity matrix both aligned well with the direction of the force generated upon binding at micropillars disregard which type of biochemical niche signal it is (FN or E-Cad) and whether the biochemical niche signals are symmetric, FN/FN or E-Cad/E-Cad (FIG. 14D, 14E, row 1 or row 2) or asymmetric, FN/E-Cad, (FIG. 14D, 14E, row 3b).

In addition to the lateral cell division along the x-y axis, perpendicular cell division in the z-axis is also important for precise tissue organization and morphogenesis. For example, during mammalian skin epidermis development, the cell division along z-axis that is perpendicular to the basement membrane assured proper stratification of skin (Lechler and Fuchs, 2005). However, little was known about how local biophysical microenvironment factors, for example the geometrical factors, could affect the cell division in the z-axis, primarily due to the lack of microfabrication technologies that could precisely control the microenvironment signals in 3D. Here, a further hypothesis was that the directional cellular tensile stress induced by localized geometrical factors upon binding to the functionalized micropillars inside the micro-niche could control the cell division direction along the z-axis. To test this hypothesis, 2 micropillars 3D micro-niche was fabricated with its two opposing micropillars located at different height inside the 3D micro-niche, as shown by the schematic diagram (FIG. 14F) and the SEM image from the top view of a complete 3D micro-niche (FIG. 14G1) and the side view of a half 3D micro-niche, which well confirmed the z-axis location of two micropillars inside the micro-niche (FIG. 14G2). The two micropillars at different height were biofunctionalized with FN before seeding mESC into the micro-niche until cell division and measuring the cell division direction after 14 hour incubation. The cell division direction was found to align well with the direction of the micro-niche geometry by connecting the z-axis center of the two biofunctionalized anchoring micropillars (dotted white line) at a mean angle of 32.75° (FIG. 14I, 14J). Quantifying the cell division directions of mESCs, a skewed distribution of the cell division direction was found, with a dominant peak of cell division direction at about 30° (FIG. 14H), which was very close to the measured direction of the two anchoring micropillars.

These results provided direct evidence on controlling the cell division direction in 3D at both the x-y plane and the z-axis through defined biophysical niche signal through the geometry and the z position of the anchoring micropillars in the 3D micro-niche.

Mathematical Modeling-Based Prediction of the Biophysical Niche Signal-Induced Nucleus Deformation Alignment and Cell Division Direction in the 3D Micro-Niche

To better understand how micropillar numbers and position in 3D micro-niche could affect the cell alignment and cell division direction, and further provide a method to predict and help design in vitro microenvironment that could precisely control cell alignment and cell division direction, a quantitative model for the 3D micro-niche system was developed by modifying previously reported models (Minc et al., 2011; Thery et al., 2007).

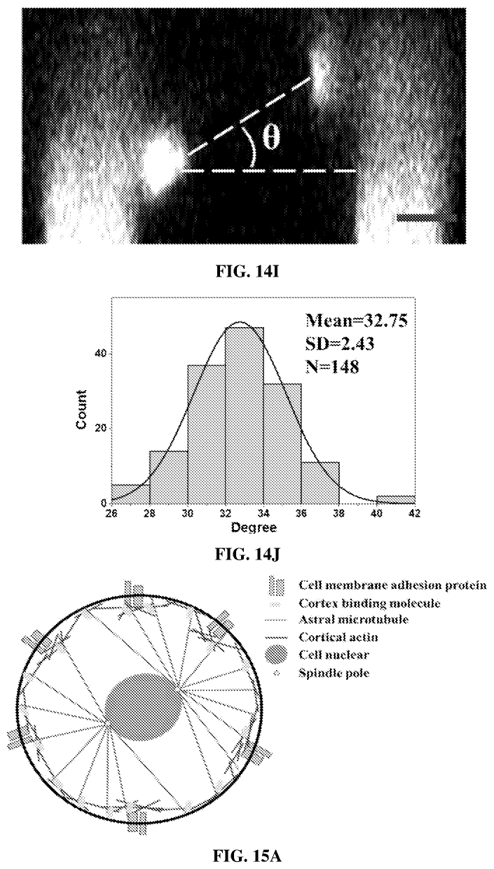

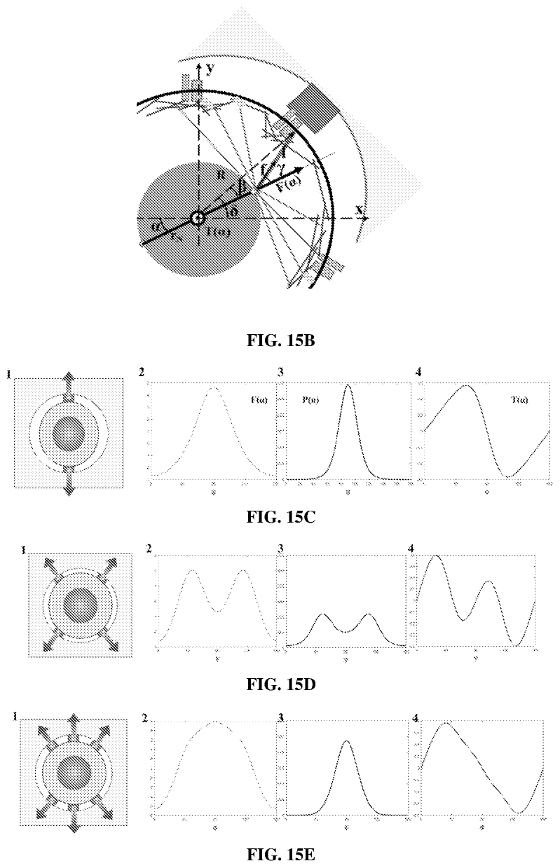

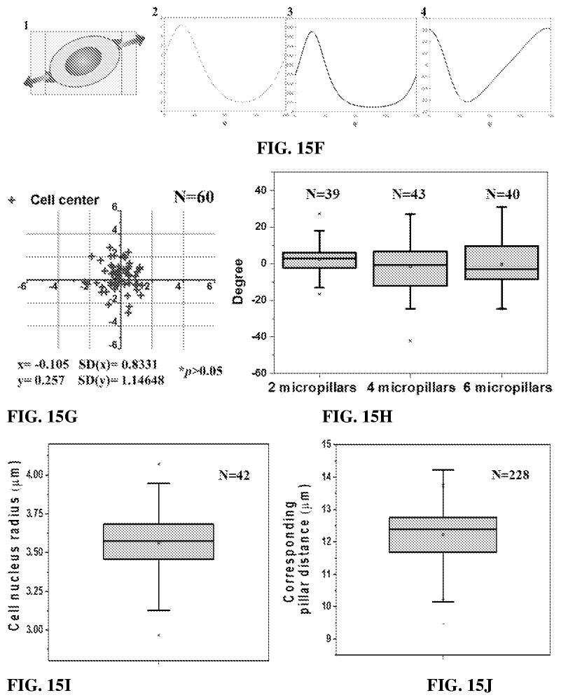

The central idea of the model is that cell adhesion, which is based on many transmembrane proteins, such as integrins, cadherins, and other cell surface glycoproteins, could transmit external microenvironment signals internally to generate intracellular cortical force. The cytoplasmic domains of these cell membrane proteins will bind to cortex binding molecules, e.g., Gai, LGN, Dynein, vimentin, and cortical actin, which pull on the astral microtubules radiating from centrosome, to control and manipulate spindle positions and through LINC, finally deforming the cell nucleus shape (FIG. 15A). In this condition, the cell nucleus acts as a force sensor, which is locally activated by cortical cues associated with cell binding and adhesion triggered by external microenvironment factors in the 3D micro-niche (FIG. 15B).

To simplify the relatively complex cell niche interactions in the micro-niche to a simple mathematical model, several assumptions were made. Firstly, the cell center is assumed to locate at the center of mass of the micro-niche. This assumption was verified through comparison between the experimental measurement of the center of gravity of the nucleus after seeding and the center of the micro-niche (FIG. 15G). Independent t-Test showed that the mean center of the nucleus was not significantly different from the center of the micro-niche (p>0.05), verifying the assumption that the cell was located at the center of the micro-niche. Secondly, microtubules bending, and spindle deformations can be neglected. Thirdly, the outside in force generated through either cell-FN or cell-E-Cad interactions is firstly transmitted to MTOC, and microtubules emanated from two MTOC around the nucleus will generate pulling force on cell nucleus through the LINC complex (Kirby and Lammerding, 2018). Finally, cell division direction was along the same x-y axis of the wall micropillars plane, which was also verified through cross section images of cell inside the 3D micro-niche (FIG. 15H and data not shown).

Here a simplified two-dimensional geometric representation of the microtubule was used (FIG. 15B). First, two forces were introduced. One is the force transmitted through microtubules, giving rise to the non-compensated force F(α), which leads to cell nucleus elongation. The second force is the torque T(α), which induces cell nucleus rotation and reorientation. The cortical force exerted per microtubule on the mitotic spindles namely f_{MT}(α,γ), where α is the angle between the nucleus axis and the x-axis, γ is the angle between the microtubule and the nucleus axis.

The non-compensated force generated by each microtubule:

f(α,δ)=f_{MT}(γ)*cos(γ), (1)

The resultant total force F(α) is obtained by summing the projected force over all microtubules:

F(α)=∫_{-π}^{π}f_{MT}(γ)*cos(γ)dγ, (2)

The torque generated by each microtubule:

τ(α,δ)=r_{N}*f_{MT}(γ)*sin(γ), (3)

where r_{N} is the distance from centrosome to the cell's center of mass along the axis α. This yields a total torque, T(α),

T(α)=r_{N}*·_{-π}^{π}f_{MT}(γ)*sin(γ)dγ, (4)

f_{MT}(γ)=ρ*f_{ad}, (5)

where

$$ρ = \frac{{N}_{M T}}{2 π} \frac{d γ}{d β}$$

is the microtubules angular density, N_{MT} is the total number of microtubules inside the cell. This model assumes that the adhesion force generated between the cell and each micropillar f_{ad} is stable and constant.

The orientation of nucleus deformation and cell division could be identified from the local minima of the effective energy landscape. The probability density function of the orientation is then calculated by introducing a white noise in the distribution of torques,

$$P ( α ) = p * exp ( - \frac{W ( α )}{d} ) \qquad ( 6 )$$

where d is a dimensionless fitting parameter that includes noise strength and nuclear friction, the effective landscape W(α) is a primitive of T(α). The parameter d is obtained through matching the simulation results to the experimental results in each condition. And p, a factor that is adjusted to make sure that the sum of the probability density P(α) is 1. Therefore, r_{N}, R, d are known variables and the only input variable is the microtubule cortical force f_{MT}(γ), which is based on the geometry of wall micropillars. Here the r_{N} is 3.57 μm through measuring the cell nucleus, R is 6.11 determined experimentally by measuring the micropillars in 3D micro-niche (FIG. 15I). The mathematical modeling results are calculated through analytical solutions. In particular, the probability density function of cell division direction P(α), the total force F(α), and the total torque T(α) were plotted (FIG. 15C-F), and as the F(α) and P(α) were directly related to the cell alignment and cell division direction, studies focused on these two variables. The distribution of the alignment of the total force F(α) generated by microtubule (FIG. 15C, 2, 15D, 2, 15E, 2, 15F, 2) showed a similar trend as the cell division direction probability density P(α) (FIG. 15C, 3, 15D, 3,15E, 3,15F, 3) in the 2, 4 and 6 micropillar micro-niche groups. This result was reasonable as the tensile stress force generated upon firm attachment to the micropillars would deform, elongate and rotate the nucleus through the movement of the microtubules and LINC until the nucleus long axis direction and tensile stress force direction align. In the 2 micropillars group (FIG. 15C, 1), both F(α) and P(α) showed a bell shape with a single major peak at 90° (FIG. 15C, 2-3) matching well with the experimental results (FIG. 14D, 1-3, 14E, 1-3), and T(α) was 0 at 0° and 180° because the symmetric elimination of torque applied to cell nucleus while at 90° no torque was applied to cell nucleus (FIG. 15C, 4). In the 4 micropillars group (FIG. 15D, 1), both F(α) and P(α) showed a typical dual peaks distribution with two major peaks at about 50° and 130° (FIG. 15D, 2-3) matching well with the experimental results (FIG. 14D, 4-6, 14E, 4-6), and 5 angles with 0 torque T(α) were observed (FIG. 15D, 4). In the 6 micropillars group (FIG. 15E, 1), F(α) and P(α) showed a bell shape with the peak at 90° but had a wider distribution (FIG. 15E, 2-3) agreeing with the experimental results (FIG. 14D, 7-9, 14E 7-9), and 3 angles with symmetric elimination of T(α) were found (FIG. 15E, 4). In the 2 micropillars with different heights micro-niche (FIG. 15F, 1), single peak distribution of F(α) and P(α) were found at about 30° (FIG. 15F, 2-3), agreeing with the experimental results (FIG. 14H), and T(α) was 0 at 0°, 90°, 180° because of the symmetric elimination (FIG. 15F, 4). All the F(α) and P(α) modeling results fitted well with experimental results, suggesting that the mathematical model successfully predicted the nucleus deformation alignment and the cell division direction and hence could serve as a useful tool to predict the responses in nucleus deformation alignment and cell division direction towards engineered cell niche.

### 4. Tensile Stress Force but not Cell Shape Determines the Cell Division Direction in the 3D Micro-Niche

The direction of cell division determines the polarity of the two daughter cells and hence the geometry and shape of the newly formed tissues. More than one century ago, Hertwig proposed that cell division direction was determined by the shape of the cell (Hertwig, 1884). While recently, evidences suggested that tensile stress might be the dominant factor that determined cell division direction. Specifically, Kevin C. Hart and co-workers showed that a very small uniaxial stretch (12%) could reorient cell division direction irrespective of the long axis of the MDCK cell monolayers (Hart et al., 2017). However, the MDCK cells in a monolayer population have random orientations and varying cell shapes. As a result, a definitive answer to the question on whether it is the tensile force or the cell shape determined the cell division direction cannot be clearly obtained, unless the cell shape and the tensile stress can be separately manipulated.

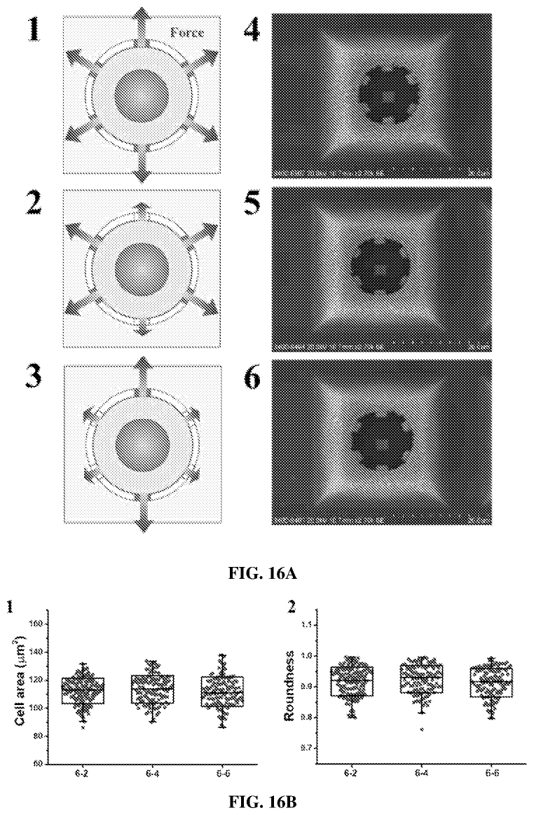

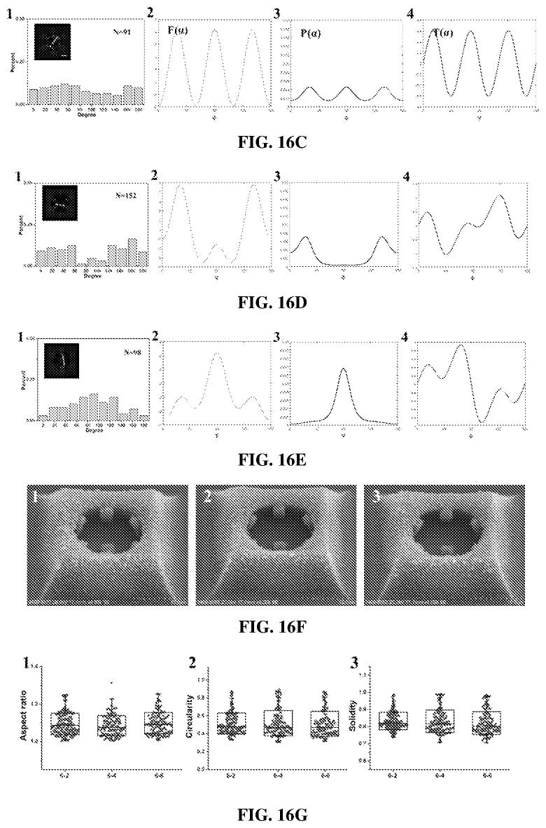

Using the MMM platform, the dimension, the topological features, the mechanical and the biochemical niche signals of protein microstructures could be precisely and independently manipulated. Specifically, 3D micro-niches with fixed dimension (28×28×20 μm) and identical topological features (6 micropillars evenly distributed with 60 degree between each other) were fabricated (FIG. 16A, 1-6, FIG. 16F, 1-3), while the 6 micropillars were selectively functionalized with a biochemical niche signal (matrix niche FN) in three differentially and spatially controlled patterns, all six micropillars (data not shown), the four diagonal micropillars, or the two pivotal micropillars (data not shown). Single mESC inside these three types of micro-niches, confocal with phase contrast images (data not shown) were found to have the same cell area, dimension and shape prior to cell division, as shown by the insignificantly changed morphological measures including area (FIG. 16B, 1), roundness (FIG. 16B, 2), aspect ratio (FIG. 16G, 1), circularity (FIG. 16G, 2) and solidity (FIG. 16G, 3), analyzed using ImageJ. The single mESC resided in the micro-niches attached to the FN-functionalized micropillars, but not the non-functionalized ones, as shown by the immune-positive staining of the markers for focal adhesions including integrin β1 (data not shown) and phosphorylated, with cell nucleus and 3D BSA micro-niche (data not shown). Together with the merged channel, activated focal adhesion signaling integrin β1 was localized proximity to the FN-functionalized micropillars but not the non-functionalized ones, which indicated higher local tensile force generation near the FN-functionalized micropillars.

Upon functional binding of the mESCs to the FN-functionalized micropillars via focal adhesion formation, intracellular tensile force was generated along the axes connecting the 6 FN-functionalized micropillars (FIG. 16C, 1), the 4 diagonal FN-functionalized micropillars (FIG. 16D, 1) and the 2 pivotal FN-functionalized micropillars (FIG. 16E, 1), as predicted by the mathematical modeling of the noncompensated force F(α) (FIG. 16C, 2, 16D, 2, 16E, 2), the probability density P(α) of cell division direction (FIG. 16C, 3, 16D, 3, 16E, 3), and the torque force T(α) experienced by cell nucleus (FIG. 16C, 4, 16D, 4, 16E, 4). Specifically, in the 6 FN-functionalized micropillar case, the cell division direction showed a random and dispersive distribution (FIG. 16C, 3), in the 4 FN-functionalized micropillar case, the cell division direction showed a dual-peak probability distribution at around 50° and 130° (FIG. 16D, 3), while in the 2 FN-functionalized micropillar case, the cell division direction showed a single peak probability distribution at about 90° (FIG. 16E, 3). Indeed, the subsequent cell division directions were in good agreement with the predicted results.

To conclude, these studies confirmed that cell adhered on FN functionalized micropillars could induce heterogenous tensile stress force inside the cell while maintaining the same cell shape and resulted in controllable cell division direction in our 3D micro-niches.

5. Asymmetric Biochemical Niche Signals in the Single Cell 3D Micro-Niche Controlled/Determined the ACD Orientation of mESC

Stem cells including mESCs are able to undergo ACD, which is a special type of cell division giving rise to two daughter cells with differential fates, one retaining the stem cell characteristics (sternness) while another differentiated or committed (sternness lost). Proper orientation of ACD is extremely important as it governs correct tissue morphogenesis, patterning and homeostasis during development. As a result, knowledge on how to, and capability to control the orientation of ACD and the subsequent cell fates, is critical for tissue engineering and regenerative medicine.

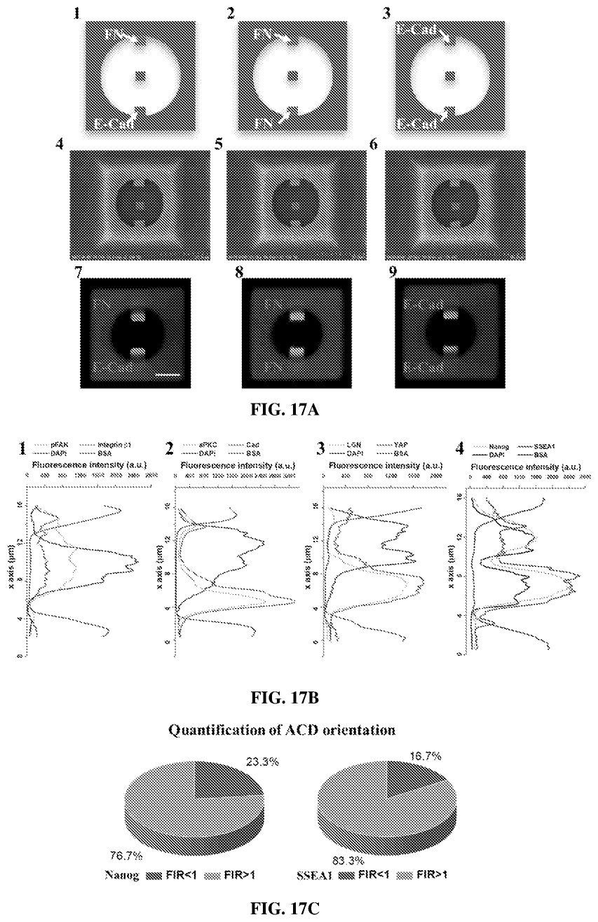

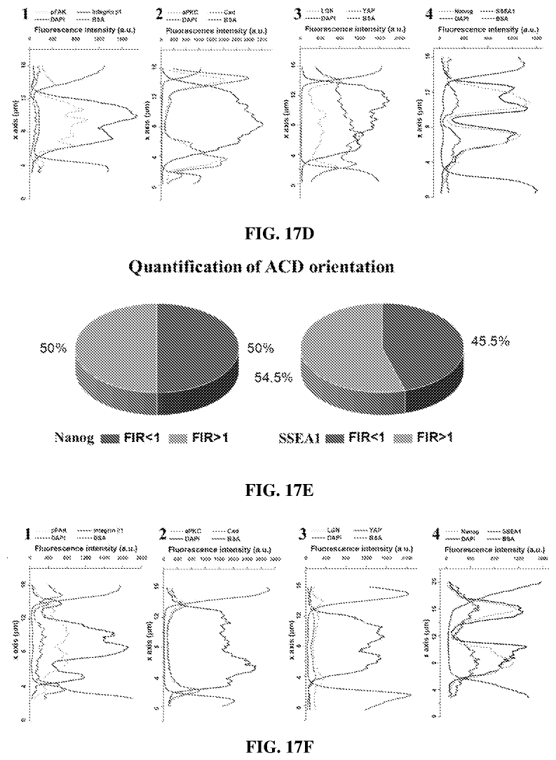

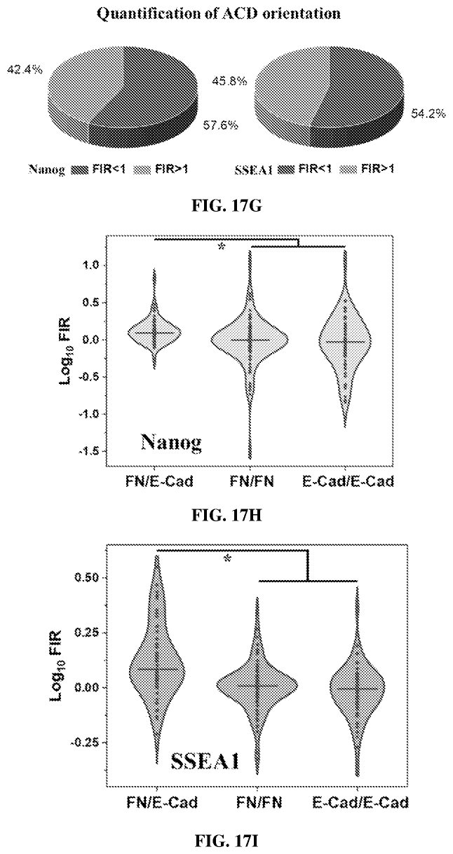

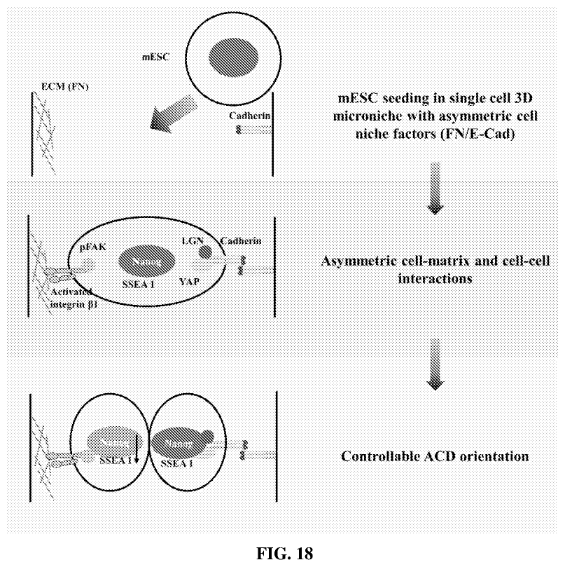

The first part of the current study demonstrated that local biophysical signals, such as the tensile force built up intracellularly in mESCs upon binding to the biofunctionalized micropillars, controlled the cell division direction. Here, a hypothesis was that, along a pre-determined cell division direction, such as that defined by the 2 axial micropillars inside the micro-niche, asymmetric biochemical niches, in particular, a matrix niche FN on the top micropillar, and a cell niche E-Cad on the bottom micropillar, shown as schematic diagram, SEM image and confocal fluorescence image (FIG. 17A, 1, 4, 7), will control the ACD orientation of mESC. The matrix niche FN was chosen because it promoted mESC differentiation through integrin-based adhesion formation and signaling while E-Cad was chosen because it associated with pluripotency maintenance of mESC (Hayashi et al., 2007; Pimton et al., 2011; Redmer et al., 2011). As controls for the asymmetric micro-niche, two micro-niches with symmetric designs, where both micropillars of the same micro-niche were functionalized with either the matrix niche FN, shown as schematic diagram, SEM image and confocal fluorescence image (FIG. 17A, 2, 5, 8) or the cell niche E-Cad, shown as schematic diagram, SEM image and confocal fluorescence image (FIG. 17A, 3, 6, 9), were fabricated. After seeding mESCs on the micro-niche arrays for 6 hours, which is well before the telophase, intracellular localization of several polarity-associated proteins including phosphorylated focal adhesion kinase (pFAK), activated integrin β1, atypical protein kinase C (aPKC), pan-cadherins, Leu-Gly-Asn-enriched protein (LGN), and yes-associated protein (YAP) (data not shown), were identified via immunofluorescence staining, using DAPI as the counter stain (data not shown) and the photo sensitizer rose bengal used during fabrication of the BSA protein micro-niche as the internal reference (data not shown). First, the most upstream player of the cell-matrix adhesion formation, the active form of integrin β1 formed tiny clusters at the proximity to the FN-functionalized micropillar, while the other member of the cell-matrix adhesion, pFAK showed intensive expression particularly at the circumferential region of the cell. Second, aPKC and pan-cadherin were highly enriched at the cell membrane in proximity to the micropillar bio-functionalized with the extracellular domain of E-Cad, suggesting the presence of interactions between the mESC-expressed cadherin and the E-Cad functionalized on the surface of the bottom micropillar inside the micro-niche. Third, two additional downstream proteins of E-Cad signaling LGN, and YAP were enriched in proximity to the E-Cad-functionalized micropillar. These results suggest that functional binding of mESC to the FN-functionalized micropillar of the micro-niche triggered the formation of integrin β1-based focal adhesions at the proximity and activated pFAK signaling subsequently, while the functional binding of mESC to the E-Cad-functionalized micropillar of the micro-niche triggered the formation of cadherin related signaling pathways. Merged fluorescence channels of the single mESCs in the first, second and third row were analyzed and used for generation of asymmetric fluorescence signal distribution of single cell inside the micro-niche (FIG. 17B, 1-3). The data (not shown) revealed the establishment of cell polarity, as shown by the asymmetric immuno-localization of cell-matrix and cell-cell interaction proteins, induced by the asymmetric biochemical niche signals, the matrix niche FN on one side, and the cell niche E-Cad on the other side, of the 3D micro-niche.

To investigate whether the asymmetric niche signals of the micro-niche (FIG. 17A, 1) and the subsequently induced cell polarity (data not shown) resulted in a biased orientation of ACD in mESC, mESCs were seeded in the micro-niches and cultured for 14 hours, which was slightly longer than the median cell cycle length of mESCs previously reported (Waisman et al., 2019). Immunofluorescence staining of two mESC stem cell markers, Nanog and SSEA1, were used to verify the stemness states of the daughter cells. Immune-positive expression of both stemness markers Nanog and SSEA1 co-localized at the intra-nuclear region of the daughter cell proximal to the E-Cad micropillar, demonstrating the stem cell identity of the self-renewed daughter cell (data not shown). On the contrary, the daughter cell close to the FN micropillar showed reduced expression of Nanog and lost the expression of SSEA1, suggesting the identity of the committed daughter cell), which indicated asymmetric inheritance of stemness between two daughter cells (data not shown). The asymmetric fluorescence signal distribution of Nanog and SSEA1 of two daughter cells inside the micro-niche further supported this conclusion (FIG. 17B, 4).

In order to quantify the difference in the level of expression of the mESC stemness markers Nanog and SSEA1 between the two daughter cells exposing to the asymmetric niche factors (FN/E-Cad) inside the micro-niche, the fluorescence intensity of the immunofluorescence staining of these markers in the daughter cell proximal to the E-Cad micropillar was divided by that of the daughter cell proximal to FN micropillar in the FN/E-Cad micro-niche. When this fluorescence intensity ratio (FIR) is greater than (>) 1, it means that the daughter cell proximal to the E-Cad micropillar is stem cell while the other cell proximal to FN micropillar is committed. If the FIR is less than (<) 1, it means the daughter cell close to the E-cad micropillar is committed, while the other one close to the FN micropillar maintains as stem cell. To verify and demonstrate controllable orientation of ACD in micro-niche system, analysis and comparison of the number of daughter cells pairs with FIR>1 was conducted, which indicated the daughter cell proximal to bottom micropillar better maintained its stem cell fate, while with FIR<1, which indicated the daughter cell proximal to the bottom micropillar lost its stemness. For Nanog, 46 out of 60 (76.7%) cells pairs were found with FIR>1, and for SSEA1, 50 out of 60 (83.3%) cells pairs were found with FIR>1 in the asymmetric FN/E-Cad group (FIG. 17C). To conclude, a significantly higher expression of Nanog and SSEA1 proteins in the cell proximal to the E-Cad micropillar than the cell proximal to the FN micropillar, which are 76.7% and 83.3% respectively (p<0.0005), indicated biased ACD was observed in the asymmetric FN/E-Cad micro-niche.

In contrast, no cell polarity was observed in the control groups including symmetric FN/FN micro-niche and E-Cad/E-Cad micro-niche (data not shown). Moreover, no significant sign of ACD could be found from either confocal fluorescence images (data not shown) or fluorescence signal distribution of single cell inside the micro-niche (FIG. 17D, 1-4, FIG. 17F, 1-4) in the symmetric FN/FN and E-Cad/E-Cad micro-niche groups. Finally, the logarithm base 10 of FIR of Nanog and SSEA1 were plotted and compared, among asymmetric FN/E-Cad micro-niche and symmetric FN/FN, E-Cad/E-Cad micro-niches. For both signals, FN/E-Cad group was significantly higher (p<0.05) than FN/FN and E-Cad/E-Cad groups (FIG. 17H, 17I). Together, these results suggested the asymmetric distribution of niche factors including FN and E-Cad successfully translated into asymmetric signaling as well as cell fates inside mESCs. These studies demonstrated the design of asymmetric distribution of two biochemical signals as a minimal model, which were FN and E-Cad, were enough to induce robust mESC polarity and oriented ACD inside the micro-niche in vitro.

Discussion

Building in vitro models recapitulating the intricate native cell microenvironment represents a reductionist approach to study the influence of individual niche factors on cell behaviors and cell fates. The defects in conventional additive manufacturing techniques, such as low 3D fabrication resolution, lack of decoupling of and independent control over multiple biochemical and biophysical factors, hinder the design and fabrication of 3D cell niche model, particularly at single cell level, for investigation of the interactions between the cells and their microenvironment. This gap is overcome by using two-photon microfabrication and micropatterning technique. The 3D micro-niche fabricated by the disclosed technique has several advantages. First, 3D microstructures fabricated by this technique has micrometer resolution and good lateral biophysical and biochemical signals controllability. Second, the time consuming for every single micro-niche is about 10-15 seconds, and the fast fabrication speed enables the possibility of constructing a high throughput system, with the possibility to generate large statistics of cellular data. Moreover, the stepwise fabrication process provides the ability to individually control all the biochemical and biophysical microenvironment factors, such as the 3D constructing of micro-niche, the control of wall micropillar number, position, and the spatial lateral crosslinking and functionalizing of biochemical signals, such as FN and E-Cad in this experiment.

After establishing this microfabrication technique, it was applied this micro-niche system to study how localized and heterogeneous biophysical and biochemical signals could affect cell behaviors and cell fates, using mESC as an example. Through controlling the wall micropillar number, z-axis position, allowed precise tuning the microenvironment geometry inside the 3D micro-niche. The results showed that cells sensed and responded to different geometry signals inside the 3D micro-niche, in terms of cell alignment and cell division direction. The results between different biofunctionalization conditions, further demonstrated that these geometrical signals provided mechanical force transmission outside-in to control cell alignment and cell division direction, in spite of the biochemical nature of cellular adhesion types, for example, the FN mediated cell-matrix interaction and E-Cad mediated cell-cell interaction.

In this study, cell alignment and cell division direction were successfully explained and modeled through a cellular cytoskeleton based cytoplasmic tensile stress force mathematical model. In this model, mESCs generated intracellular tensile stress force outside-in through cellular interaction with the external geometrical microenvironment factors, which were the biofunctionalized micropillars. The tensile stress force was transduced through astral microtubules to the spindle poles, which generated noncompensated force as well as the torque to the cell nucleus. This model fitted well with the experimental results and successfully explained why the change of the number of micropillars could affect the cell alignment and cell division direction Similar results were found in several studies which are based on the micropatterned surface made of different types of cell adhesion proteins using conventional 2D microfabrication techniques, such as microcontact printing, and showed cell adhesion based force were important in cell alignment, spindle orientation, and finally cell division in fibroblast (Fink et al., 2011; Freida et al., 2013; Thery et al., 2007). Our results further proved the same mechanism and model worked in 3D micro-niche as well.

A scientific question for a long time about what is the crucial factor for cell division direction determination, is it the cell shape or intracellular tensile stress force? Prior studies have used homogenously biofunctionalized biomaterial geometry, thus cannot induce differential cellular tensile stress force with the same geometry and cell shape. The current studies overcome this problem using our 3D micro-niche system. Here, cells were physically confined in the six micropillars micro-niches, which meant the same micro-niche geometry, with the same cell shape but generated different tensile stress force patterns through the combination of FN biofunctionalized and non-functionalized micropillars. The data directly proved that intracellular tensile force but not the cell shape determined cell division direction. The 3D micro-niche together with the mathematical model is a powerful toolkit that would serve as a good method for studying how single cells can interact with their local biophysical microenvironment.

To conclude, a precisely engineered and tunable, protein-based single cell 3D micro-niche with heterogenous cell niche factors was successfully fabricated and used to study how localized cell niche signals affect and manipulate cell behaviors and cell fates particularly cell division direction and orientation of ACD, using mESCs as an example

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

### REFERENCES

Chan, et al. Advanced Functional Materials, 24(3), 277-294 (2014).
Huang, N et al. Advanced Biosystems, 2(8), 1800053 (2018).
Kukumberg, et al. Biomaterials, 131, 68-85. (2017)
Ma, et al. ACS Appl. Mat & interfaces, 9(35), 29469-29480 (2017).
Tong, et al. Scientific reports, 7(1), 12402 (2017).
Tong, et al. Scientific reports, 6, 20063 (2017).
Wang, X., Gao, B., & Chan, B. P. Multiphoton Microfabrication and Micropatterning (MMM)—An All-In-One Platform for Engineering Biomimetic Soluble Cell Niches. Biomaterials, 120644.

1. A biochip comprising a first and a second layer, wherein:
(i) the first layer is a macrostructure comprising a solid support,
wherein the solid support comprises a flat top surface or a top surface comprising a plurality of microwells, and
(ii) the second layer comprises one or more microstructures comprising a bioactive factor and/or one or more cell niche factors selected from topological factors and biofactors,
wherein the one or more topological factors is selected from the group consisting of a flat micro-matrix, a pillar array, a fiber-bead microstructure, a grating, a convex, a concave, a cave, a wavy structure, and combinations thereof, and
wherein the one or more biofactors are selected from the group consisting of an extracellular matrix (ECM) macromolecule, a cell-cell interaction molecule and/or a soluble factor.

2. The biochip of claim 1, wherein the flat top surface of the solid support is compartmentalized into microwells using a removable inner isolator.

3. The biochip of claim 2, further comprising a removable outer isolator and a removable inner isolator and/or wherein the flat top surface of the solid support comprises a marking.

4. (canceled)

5. The biochip of claim 1, wherein the top surface of the solid support comprises a plurality of microwells, in a microplate format, wherein the microwells comprise the one or more microstructures, wherein the microplate format is selected from preferably, in a 6, 12, 24, 48, 96, 384 or 1536 wells.

6. The biochip of claim 1, wherein: (a) the removable inner isolator comprises microwells between about 0.5 to about 10 mm in height, or biochip of claim 1, wherein the microwells are between about 0.5 to about 10 mm in height; (b) wherein the second layer has a height of about 0.2 μm to about 100 μm, preferably 20 μm; (c) wherein the ECM molecule is selected from the group consisting of collagen 1, 2, 4 or 6, vitronectin, fibrinogen, laminin 411, 511, or 521, Thrombospondin, tenacin, mucin, byglycan, aggrecan, and decorin; or (d) the soluble factor is selected from the group consisting of BMP2, Wnt3a, EGF, bFGF, TGF-β, BMP4, WNt5A, IL-2 and IL-18.

7. (canceled)

8. (canceled)

9. (canceled)

10. The biochip of claim 1, comprising a biomaterial substrate comprising bovine serum albumin, human serum albumin, or collagens, with a height between about 1 and 50 μm, preferably, about 5 μm; and/or one or more soluble factors or cell adhesion molecules (CAM), wherein the soluble factor or CAM is conjugated to one member of an affinity binding pair.

11. (canceled)

12. The biochip of claim 1, wherein: (a) the soluble protein or CAM molecule is incorporated onto the microstructure via affinity interactions between an affinity pair selected from the group consisting of extracellular matrix (ECM), growth factor, albumin binding domain (ABD)-serum albumin (SA), barnase-barster), biotin-avidin, Fc-protein A/G, and His-nickel nitrilotriacetic acid (Ni-NTA), and optionally, wherein the soluble protein is conjugated to biotin; and/or (b) the CAM is a fusion protein comprising an FC domain.

13. (canceled)

14. (canceled)

15. A single cell 3D micro-niche biochip comprising a solid substrate on which an inert protein is deposited, and functionalized with a combination of factors specific for a microenvironment of a single cell, wherein optionally, the single cell is a stem cell, such as embryonic stem cell, mesenchymal stem cell, or iPSC.

16. The single cell 3D micro-niche biochip of claim 15, (a) comprising protein micropillars functionalized with a soluble protein, preferably the soluble protein is selected from an ECM, fibronectin (FN), a cell-cell interaction molecule, E-Cadherin (E-Cad), or a combination thereof, optionally, wherein the soluble proteins and/or cell-attachment proteins are attached to the substrate using an indirect affinity-binding pair; (b) comprising micropillars evenly distributed with 60 degree between each other, wherein the micropillars are functionalized with a biochemical niche signal (matrix niche FN); (c) wherein a cell-cell adhesion molecule is immobilized on the substrate using a laser in the presence of a fluorescence tagged E-Cad-Fc at a concentration of 25 μg/ml; and/or (d) comprising micro niche dimensions selected from:
a micro-niche outer wall length of about 25-45 μm, preferably between about 28 and 37 μm and the height of about 20 μm;
an inner aperture diameter of about 10-25 μm, preferably, about 15-20 μm;
a distance between corresponding pillars can be about 8-15 μm, preferably between about 10-12 μm;
a z-axis distribution from about 10 to 13 μm from glass surface;
or a combination thereof.

17. (canceled)

18. (canceled)

19. (canceled)

20. (canceled)

21. A cell micro-niche screening method comprising two phases, wherein the first phase uses individual micro niche factors, and the second phase uses combination of niche factors selected after the first phase wherein the first phase comprises:
microfabricating microstructures incorporating individual niche factors identified as relevant for a particular test cell type/types in vivo
culturing the test cell/cells on a substrate comprising microstructures incorporating the individual niche factors and evaluating phenotypic endpoints selected from morphology and marker expression specific for the test cell/cells and identifying niche factors that maintain the phenotypic endpoints as phenotype-maintaining cell niche factors,
wherein the second phase comprises microfabricating microstructures integrating the phenotype-maintaining cell niche factors identified from the first stage in combination, and culturing the test cell/cells on a substrate comprising microstructures incorporating the combination of the phenotype-maintaining cell niche factors using.

22. The method of claim 21, wherein: (a) the microstructures for the first phase are microfabricated in a biochip design comprising microwells, wherein each microwell comprises only one test cell niche factor; and/or (b) the phenotype-maintaining cell niche factors are layered at the bottom of microwells.

23. (canceled)

24. The method of claim 21, wherein: (a) the cell niche factors are selected from the group consisting of extracellular matrix proteins, cell-cell adhesion proteins, cellular proteins, mechanical factors selecting from elastic modulus, stiffness and active force and topological factors selected from the group consisting of as flat matrix (BSA/FM), micro-pillar array (MPA), fiber-bead microstructure (FB), thick grating (TkG), thin grating (TnG), parallel grating hierarchy (GHpl), perpendicular grating hierarchy (GHpp), convex (Cv), and concave (Cc); and/or (b) the ECM proteins are selected from the group consisting of collagen 1, 2, 4 or 6, vitronectin, fibrinogen, laminin 411, 511, or 521, Thrombospondin, tenacin, mucin, byglycan, aggrecan, and decorin, and/or the soluble factor is selected from the group consisting of BMP2, Wnt3a, EGF, bFGF, TGF-β, BMP4, WNt5A, IL-2 and IL-18; and/or (c) the microstructures incorporating cell niche factors are prepared using the multiphoton micropatterning and microfabrication platform to arbitrarily control the various niche properties, including one or more of mechanical, topological, and biochemical properties, by an iterative fabrication approach.

25. (canceled)

26. (canceled)

27. A method for microfabrication and micropatterning of bioactive soluble factors and/or cell-cell adhesion molecules, comprising:
(a) fabricating a biomaterial substrate comprising proteins or polymers with a pre-designed micro-structure, on a supporting surface;
micropatterning a layer of a linker material, on the biomaterial substrate;
conjugating a specific binding partner of the linker material on a bioactive soluble factor or a cell-cell adhesion molecule; and
micropatterning the bioactive soluble factor or the cell-cell adhesion molecule onto the micropatterned linker material through functional binding with the specific binding partner; or
(b) fabricating a protein substrate micro-structure on a supporting surface;
micropatterning a layer of linker material on the protein substrate;
conjugating a bioactive soluble factor/cell-cell adhesion molecule with a specific binding partner of the linker material; and
micropatterning the bioactive soluble factor conjugated with the binding partner of the linker material through functional binding onto the micropatterned layer of linker material.

28. The method of claim 27, wherein: (a) the bioactive soluble factor comprises a cytokine, a growth factor, and/or an enzyme; (b) the biomaterial substrate consists of a protein, optionally the biomaterial substrate consists of serum albumin or collagens; (c) the pre-designed micro-structure comprises a structure selected from the group consisting of a flat matrix, a micro-pillar array, a fiber-bead microstructure, a grating, a convex, a concave, or combinations thereof; (d) the linker material is avidin and its specific binding partner is biotin; and/or (e) both the microfabrication of the biomaterial substrate and the micropatterning of the linker material are achieved through multiphoton laser; and optionally, wherein the bioactive soluble factor is selected from the group consisting of BMP2, Wnt3a, EGF, bFGF, TGF-β, BMP4, WNt5A, IL-2 and IL-18.

29. (canceled)

30. (canceled)

31. (canceled)

32. (canceled)

33. (canceled)

34. The method of claim 27, wherein the local density of the biomaterial substrate, the linker material and hence the bioactive soluble factor microfabricated and micropatterned in claim 26 is quantitatively controlled by parameters selected from:
laser power, ranges from 1 to 250 mW, preferably 45 mW;
laser scan cycle, ranges from 1 to 100 cycles, preferably 11 cycles;
concentration of linker material, ranges from 0.5 to 20 mg/ml, preferably 9 mg/ml; molar ratio of the binding partner to the bioactive soluble factors, ranges from 0.5:1 to 50:1, preferably 5:1; and/or
an amount of binding partner-conjugated soluble factor applied to the linker material micropatterns, ranges from 10 to 1000 ng, preferably 500 ng.

35. A biomimetic soluble cell niche biochip made according to the method of claim 27.

36. The method of claim 27, comprising:
fabricating a protein substrate micro-structure on a supporting surface;
micropatterning a layer of linker material on the protein substrate;
conjugating a bioactive soluble factor/cell-cell adhesion molecule with a specific binding partner of the linker material; and
micropatterning the bioactive soluble factor conjugated with the binding partner of the linker material through functional binding onto the micropatterned layer of linker material.

37. The method of claim 36, wherein: (a) the soluble cell niche is a bioactive soluble factor; and/or (b) the protein substrate comprises serum albumin, fibronectin, gelatin, laminin, histone, fibrinogen, collagen, or a combination thereof; and wherein the supporting surface is provided by a glass, a silicon, a quartz or a plastic; (c) the linker material and its binding partner form a host-guest binding pair, preferably the pair is selected from biotin-avidin, albumin binding domain (ABD)-serum albumin (SA), barnase-barster, Fc-protein A/G, and His-nickel nitrilotriacetic acid (Ni-NTA); and/or (d) the local concentration of the bioactive soluble factor in the soluble cell niche biochip is controlled using the following:
laser power, ranges from 1 to 250 mW, preferably 45 mW;
laser scan cycle, ranges from 1 to 100 cycles, preferably 11 cycles;
concentration of linker material, ranges from 0.5 to 20 mg/ml⁻¹, preferably 9 mg/ml⁻¹;
molar ratio of the binding partner to the bioactive soluble factors, ranges from 0.5:1 to 50:1, preferably 5:1; and/or
amount of binding partner-conjugated soluble factor applied to the linker material micropatterns, ranges from 10 to 1000 ng, preferably 500 ng.

38. (canceled)

39. (canceled)

40. (canceled)

41. A method of manipulating the cell fate of a cell or cells comprising contacting the cell or cells with the biochip of claim 1, or a single cell 3D micro-niche biochip of comprising a solid substrate on which an inert protein is deposited, and functionalized with a combination of factors specific for a microenvironment of a single cell, wherein optionally, the single cell is a stem cell, such as embryonic stem cell, mesenchymal stem cell, or iPSC,
for an effective amount of time to bind to microstructures and culturing the cells on the biochip, in a cell culture medium.

42. The method of claim 41, wherein: (a) the biochip comprises symmetrical biochemical niche factors or (b) biochip comprises asymmetrical biochemical niche factors, optionally the asymmetric niche factors include at least one factor selected from cell-cell adhesion molecules and one factor selected from ECM molecules.

43. (canceled)
